SEQUENCE LISTING (82 sequences)

SEQ ID NO: 1 - DNA, 44 bp, synthetic construct
  misc_feature 1..44: note = Description of Artificial Sequence: Synthetic oligonucleotide
  misc_feature 1..44: note = Description of Combined DNA/RNA Molecule: Synthetic oligonucleotide
  source 1..44: mol_type = other DNA
cgaactgaaaagcccactggacatataactgttgggaagggcga

SEQ ID NO: 2 - DNA, 22 bp, synthetic construct
  misc_feature 1..22: note = Description of Artificial Sequence: Synthetic oligonucleotide
  source 1..22: mol_type = other DNA
cgtattaccgcctttgagtgag

SEQ ID NO: 3 - DNA, 22 bp, synthetic construct
  misc_feature 1..22: note = Description of Artificial Sequence: Synthetic oligonucleotide
  source 1..22: mol_type = other DNA
gcgcggtattatcccgtattga

SEQ ID NO: 4 - DNA, 19 bp, synthetic construct
  misc_feature 1..19: note = Description of Artificial Sequence: Synthetic oligonucleotide
  source 1..19: mol_type = other DNA
tgaaacgcttgctgcaacg

SEQ ID NO: 5 - DNA, 23 bp, synthetic construct
  misc_feature 1..23: note = Description of Artificial Sequence: Synthetic oligonucleotide
  source 1..23: mol_type = other DNA
gtacaaaagcggtgttcgcaatc

SEQ ID NO: 6 - DNA, 25 bp, synthetic construct
  misc_feature 1..25: note = Description of Artificial Sequence: Synthetic oligonucleotide
  source 1..25: mol_type = other DNA
gttacccaacttaatcgccttgcag

SEQ ID NO: 7 - DNA, 19 bp, synthetic construct
  misc_feature 1..19: note = Description of Artificial Sequence: Synthetic oligonucleotide
  source 1..19: mol_type = other DNA
tgcgtattgggcgctcttc

SEQ ID NO: 8 - RNA, 100 nt, synthetic construct
  misc_feature 1..100: note = Description of Artificial Sequence: Synthetic polynucleotide
  source 1..100: mol_type = other RNA
gaaaagcccactggacagtcgttttagagctagaaatagcaagttaaaataaggctagtccgttatcaacttgaaaaagtggcaccgagtcggtgctttt

SEQ ID NO: 9 - RNA, 120 nt, synthetic construct
  misc_feature 1..120: note = Description of Artificial Sequence: Synthetic polynucleotide
  source 1..120: mol_type = other RNA
gaaaagcccactggacagtcgttttagagctagaaatagcaagttaaaataaggctagtccgttatcaacttgaaaaagtggcaccgagtcggtgcgctagttattgctcaggcccgttt

SEQ ID NO: 10 - DNA, 260 bp, synthetic construct
  misc_feature 1..260: note = Description of Artificial Sequence: Synthetic oligonucleotide
  misc_feature 1..260: note = Description of Combined DNA/RNA Molecule: Synthetic oligonucleotide
  source 1..260: mol_type = other DNA
aagctaatacgactcactataggaaaagcccactggacagtcgttttagagctagaaatagcaagttaaaataaggctagtccgttatcaacttgaaaaagtggcaccgagtcggtgcgctagttattgctcaggcccgttttgaaagcccactggacagtcgttttagagctagaaatagcaagttaaaataaggctagtccgttatcaacttgaaaaagtggcaccgagtcggtgcgctagttattgctcaggcccgt

SEQ ID NO: 11 - DNA, 121 bp, synthetic construct
  misc_feature 1..121: note = Description of Artificial Sequence: Synthetic polynucleotide
  source 1..121: mol_type = other DNA
aactgttgggaagggcgatcggtgcgggcctcttcgctattacgccagctggcgaaagggggatgtgctgcaaggcgattaagttgggtaacgccagggtcgggcctgagcaataactagc

SEQ ID NO: 12 - DNA, 129 bp, synthetic construct
  misc_feature 1..129: note = Description of Artificial Sequence: Synthetic polynucleotide
  source 1..129: mol_type = other DNA
accctggcgttacccaacttaatcgccttgcagcacatccccctttcgccagctggcgtaatagcgaagaggcccgcaccgatcgcccttcccaacagtttttttttgtccagtgggcttttcagttcg

SEQ ID NO: 13 - DNA, 18 bp, synthetic construct
  misc_feature 1..18: note = Description of Artificial Sequence: Synthetic oligonucleotide
  source 1..18: mol_type = other DNA
gaagcggcggcaatacgt

SEQ ID NO: 14 - protein, 1368 aa, Streptococcus pyogenes [sequence omitted]

SEQ ID NO: 15 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 16 - protein, 586 aa, synthetic construct
  REGION 1..586: note = Description of Artificial Sequence: Synthetic polypeptide
EGEKPLAGMDFAIADSVTDEMLADKAALVVEVVGDNYHHAPIVGIALANERGRFFLRPETALADPKFLAWLGDETKKKTMFDSKRAAVALKWKGIELRGVVFDLLLAAYLLDPAQAAGDVAAVAKMHQYEAVRSDEAVYGKGAKRTVPDEPTLAEHLVRKAAAIWALEEPLMDELRRNEQDRLLTELEQPLAGILANMEFTGVKVDTKRLEQMGAELTEQLQAVERRIYELAGQEFNINSPKQLGTVLFDKLQLPVLKKTKTGYSTSADVLEKLAPHHEIVEHILHYRQLGKLQSTYIEGLLKVVHPVTGKVHTMFNQALTQTGRLSSVEPNLQNIPIRLEEGRKIRQAFVPSEPDWLIFAADYSQIELRVLAHIAEDDNLIEAFRRGLDIHTKTAMDIFHVSEEDVTANMRRQAKAVNFGIVYGISDYGLAQNLNITRKEAAEFIERYFASFPGVKQYMDNIVQEAKQKGYVTTLLHRRRYLPDITSRNFNVRSFAERTAMNTPIQGSAADIIKKAMIDLSVRLREERLQARLLLQVHDELILEAPKEEIERLCRLVPEVMEQAVTLRVPLKVDYHYGPTWYDAK

SEQ ID NO: 17 - protein, 15 aa, synthetic construct
  REGION 1..15: note = Description of Artificial Sequence: Synthetic peptide
AHIVMVDAYKPTKKG

SEQ ID NO: 18 - protein, 129 aa, synthetic construct
  REGION 1..129: note = Description of Artificial Sequence: Synthetic polypeptide
DYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 19 - protein, 313 aa, Vaccinia virus
RALFYKDGKLFTDNNFLNPVSDDNPAYEVLQHVKIPTHLTDVVVYEQTWEEALTRLIFVGSDSKGRRQYFYGKMHVQNRNAKRDRIFVRVYNVMKRINCFINKNIKKSSTDSNYQLAVFMLMETMFFIRFGKMKYLKENETVGLLTLKNKHIEISPDEIVIKFVGKDKVSHEFVVHKSNRLYKPLLKLTDDSSPEEFLFNKLSERKVYECIKQFGIRIKDLRTYGVNYTFLYNFWTNVKSISPLPSPKKLIALTIKQTAEVVGHTPSISKRAYMATTILEMVKDKNFLDVVSKTTFDEFLSIVVDHVKSSTDG

SEQ ID NO: 20 - protein, 653 aa, Escherichia coli
MRLFIAEKPSLARAIADVLPKPHRKGDGFIECGNGQVVTWCIGHLLEQAQPDAYDSRYARWNLADLPIVPEKWQLQPRPSVTKQLNVIKRFLHEASEIVHAGDPDREGQLLVDEVLDYLQLAPEKRQQVQRCLINDLNPQAVERAIDRLRSNSEFVPLCVSALARARADWLYGINMTRAYTILGRNAGYQGVLSVGRVQTPVLGLVVRRDEEIENFVAKDFFEVKAHIVTPADERFTAIWQPSEACEPYQDEEGRLLHRPLAEHVVNRISGQPAIVTSYNDKRESESAPLPFSLSALQIEAAKRFGLSAQNVLDICQKLYETHKLITYPRSDCRYLPEEHFAGRHAVMNAISVHAPDLLPQPVVDPDIRNRCWDDKKVDAHHAIIPTARSSAINLTENEAKVYNLIARQYLMQFCPDAVFRKCVIELDIAKGKFVAKARFLAEAGWRTLLGSKERDEENDGTPLPVVAKGDELLCEKGEVVERQTQPPRHFTDATLLSAMTGIARFVQDKDLKKILRATDGLGTEATRAGIIELLFKRGFLTKKGRYIHSTDAGKALFHSLPEMATRPDMTAHWESVLTQISEKQCRYQDFMQPLVGTLYQLIDQAKRTPVRQFRGIVAPGSGGSADKKKAAPRKRSAKKSPPADEVGSGAIA

SEQ ID NO: 21 - protein, 865 aa, Escherichia coli
MGKALVIVESPAKAKTINKYLGSDYVVKSSVGHIRDLPTSGSAAKKSADSTSTKTAKKPKKDERGALVNRMGVDPWHNWEAHYEVLPGKEKVVSELKQLAEKADHIYLATDLDREGEAIAWHLREVIGGDDARYSRVVFNEITKNAIRQAFNKPGELNIDRVNAQQARRFMDRVVGYMVSPLLWKKIARGLSAGRVQSVAVRLVVEREREIKAFVPEEFWEVDASTTTPSGEALALQVTHQNDKPFRPVNKEQTQAAVSLLEKARYSVLEREDKPTTSKPGAPFITSTLQQAASTRLGFGVKKTMMMAQRLYEAGYITYMRTDSTNLSQDAVNMVRGYISDNFGKKYLPESPNQYASKENSQEAHEAIRPSDVNVMAESLKDMEADAQKLYQLIWRQFVACQMTPAKYDSTTLTVGAGDFRLKARGRILRFDGWTKVMPALRKGDEDRILPAVNKGDALTLVELTPAQHFTKPPARFSEASLVKELEKRGIGRPSTYASIISTIQDRGYVRVENRRFYAEKMGEIVTDRLEENFRELMNYDFTAQMENSLDQVANHEAEWKAVLDHFFSDFTQQLDKAEKDPEEGGMRPNQMVLTSIDCPTCGRKMGIRTASTGVFLGCSGYALPPKERCKTTINLVPENEVLNVLEGEDAETNALRAKRRCPKCGTAMDSYLIDPKRKLHVCGNNPTCDGYEIEEGEFRIKGYDGPIVECEKCGSEMHLKMGRFGKYMACTNEECKNTRKILRNGEVAPPKEDPVPLPELPCEKSDAYFVLRDGAAGVFLAANTFPKSRETRAPLVEELYRFRDRLPEKLRYLADAPQQDPEGNKTMVRFSRKTKQQYVSSEKDGKATGWSAFYVDGKWVEGKK

SEQ ID NO: 22 - protein, 553 aa, Escherichia coli
MLAQLTISNFAIVRELEIDFHSGMTVITGETGAGKSIAIDALGLCLGGRAEADMVRTGAARADLCARFSLKDTPAALRWLEENQLEDGHECLLRRVISSDGRSRGFINGTAVPLSQLRELGQLLIQIHGQHAHQLLTKPEHQKFLLDGYANETSLLQEMTARYQLWHQSCRDLAHHQQLSQERAARAELLQYQLKELNEFNPQPGEFEQIDEEYKRLANSGQLLTTSQNALALMADGEDANLQSQLYTAKQLVSELIGMDSKLSGVLDMLEEATIQIAEASDELRHYCDRLDLDPNRLFELEQRISKQISLARKHHVSPEALPQYYQSLLEEQQQLDDQADSQETLALAVTKHHQQALEIARALHQQRQQYAEELAQLITDSMHALSMPHGQFTIDVKFDEHHLGADGADRIEFRVTTNPGQPMQPIAKVASGGELSRIALAIQVITARKMETPALIFDEVDVGISGPTAAVVGKLLRQLGESTQVMCVTHLPQVAGCGHQHYFVSKETDGAMTETHMQSLNKKARLQELARLLGGSEVTRNTLANAKELLAA

SEQ ID NO: 23 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 24 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 25 - protein, 898 aa, Homo sapiens
MARRRLPDRPPNGIGAGERPRLVPRPINVQDSVNRLTKPFRVPYKNTHIPPAAGRIATGSDNIVGGRSLRKRSATVCYSGLDINADEAEYNSQDISFSQLTKRRKDALSAQRLAKDPTRLSHIQYTLRRSFTVPIKGYVQRHSLPLTLGMKKKITPEPRPLHDPTDEFAIVLYDPSVDGEMIVHDTSMDNKEEESKKMIKSTQEKDNINKEKNSQEERPTQRIGRHPALMTNGVRNKPLRELLGDSENSAENKKKFASVPVVIDPKLAKILRPHQVEGVRFLYRCVTGLVMKDYLEAEAFNTSSEDPLKSDEKALTESQKTEQNNRGAYGCIMADEMGLGKTLQCIALMWTLLRQGPQGKRLIDKCIIVCPSSLVNNWANELIKWLGPNTLTPLAVDGKKSSMGGGNTTVSQAIHAWAQAQGRNIVKPVLIISYETLRRNVDQLKNCNVGLMLADEGHRLKNGDSLTFTALDSISCPRRVILSGTPIQNDLSEYFALLSFSNPGLLGSRAEFRKNFENPILRGRDADATDKEITKGEAQLQKLSTIVSKFIIRRTNDILAKYLPCKYEHVIFVNLKPLQNELYNKLIKSREVKKVVKGVGGSQPLRAIGILKKLCNHPNLLNFEDEFDDEDDLELPDDYNMPGSKARDVQTKYSAKFSILERFLHKIKTESDDKIVLISNYTQTLDLIEKMCRYKHYSAVRLDGTMSINKRQKLVDRFNDPEGQEFIFLLSSKAGGCGINLIGANRLILMDPDWNPAADQQALARVWRDGQKKDCFIYRFISTGTIEEKIFQRQSMKMSLSSCVVDAKEDVERLFSSDNLRQLFQKNENTICETHETYHCKRCNAQGKQLKRAPAMLYGDATTWNHLNHDALEKTNDHLLKNEHHYNDISFAFQYISH

SEQ ID NO: 26 - protein, 1988 aa, synthetic construct
  REGION 1..1988: note = Description of Artificial Sequence: Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGGSGGGGSGGGGSEGEKPLAGMDFAIADSVTDEMLADKAALVVEVVGDNYHHAPIVGIALANERGRFFLRPETALADPKFLAWLGDETKKKTMFDSKRAAVALKWKGIELRGVVFDLLLAAYLLDPAQAAGDVAAVAKMHQYEAVRSDEAVYGKGAKRTVPDEPTLAEHLVRKAAAIWALEEPLMDELRRNEQDRLLTELEQPLAGILANMEFTGVKVDTKRLEQMGAELTEQLQAVERRIYELAGQEFNINSPKQLGTVLFDKLQLPVLKKTKTGYSTSADVLEKLAPHHEIVEHILHYRQLGKLQSTYIEGLLKVVHPVTGKVHTMFNQALTQTGRLSSVEPNLQNIPIRLEEGRKIRQAFVPSEPDWLIFAADYSQIELRVLAHIAEDDNLIEAFRRGLDIHTKTAMDIFHVSEEDVTANMRRQAKAVNFGIVYGISDYGLAQNLNITRKEAAEFIERYFASFPGVKQYMDNIVQEAKQKGYVTTLLHRRRYLPDITSRNFNVRSFAERTAMNTPIQGSAADIIKKAMIDLSVRLREERLQARLLLQVHDELILEAPKEEIERLCRLVPEVMEQAVTLRVPLKVDYHYGPTWYDAKEGADPKKKRKVDPKKKRKV

SEQ ID NO: 27 - DNA, 1057 bp, synthetic construct
  misc_feature 1..1057: note = Description of Artificial Sequence: Synthetic polynucleotide
  source 1..1057: mol_type = other DNA
agagttgctaaaattgggcaaaagatcatcattagaagagttacaaagtggtggagggagtatggtcagcaagggagcagagttgtttacaggaattgtgcctattttgatcgagcttaatggtgatgtcaatggtcacaagttcagcgtctctggagagggcgaaggggatgccacatacggtaagcttacgttaaaattcatttgcacaacaggcaagttacctgtaccatggcccacacttgtgaccaccttaagttacggggttcagtgcttctctaggtaccctgatcacatgaagcagcacgatttcttcaagtctgccatgcctgagggatacattcaggagagaaccatattcttcgaggatgacgggaactacaagagcagggctgaggtcaagttcgaaggggatacactggtaaataggatcgagttgaccggcacagatttcaaggaagatggaaacatcctgggcaataagatggagtacaactataacgcccacaatgtctacataatgaccgataaagctaagaatggcataaaggttaatttcaagattaggcacaacatagaggatgggtccgtgcagcttgccgatcactaccagcagaatacgcccataggagatggacctgtgttgttacctgataaccactatttaagtactcagagtgctttatcaaaggaccctaacgaaaagagggatcacatgatctacttcgggttcgtcacagcggcagcaataacccacggcatggatgagctttacaagtaaggagggggattgttaccggtaaaagaagctgaggataaactcagcataaatgatccgctttttgagaggcagtggcacttggtcaatccaagttttcctggcagtgatataaatgttcttgatctgtggtacaataatattacaggcgcaggggtcgtggctgccattgttgatgatggccttgactacgaaaatgaagacttgaaggataatttttgcgctgaaggttcttgggatttcaacgacaatcgggcctgagcaataactagcaaaaaa

SEQ ID NO: 28 - DNA, 1070 bp, synthetic construct
  misc_feature 1..1070: note = Description of Artificial Sequence: Synthetic polynucleotide
  source 1..1070: mol_type = other DNA
agagttgctaaaattgggcaaaagatcatcattagaagagttacagggggggaggtggaagtggtggagggagtatggtcagcaagggagcagagttgtttacaggaattgtgcctattttgatcgagcttaatggtgatgtcaatggtcacaagttcagcgtctctggagagggcgaaggggatgccacatacggtaagcttacgttaaaattcatttgcacaacaggcaagttacctgtaccatggcccacacttgtgaccaccttaagttacggggttcagtgcttctctaggtaccctgatcacatgaagcagcacgatttcttcaagtctgccatgcctgagggatacattcaggagagaaccatattcttcgaggatgacgggaactacaagagcagggctgaggtcaagttcgaaggggatacactggtaaataggatcgagttgaccggcacagatttcaaggaagatggaaacatcctgggcaataagatggagtacaactataacgcccacaatgtctacataatgaccgataaagctaagaatggcataaaggttaatttcaagattaggcacaacatagaggatgggtccgtgcagcttgccgatcactaccagcagaatacgcccataggagatggacctgtgttgttacctgataaccactatttaagtactcagagtgctttatcaaaggaccctaacgaaaagagggatcacatgatctacttcgggttcgtcacagcggcagcaataacccacggcatggatgagctttacaagtaaggagggggattgttaccggtaaaagaagctgaggataaactcagcataaatgatccgctttttgagaggcagtggcacttggtcaatccaagttttcctggcagtgatataaatgttcttgatctgtggtacaataatattacaggcgcaggggtcgtggctgccattgttgatgatggccttgactacgaaaatgaagacttgaaggataatttttgcgctgaaggttcttgggatttcaacgacaatcgggcctgagcaataactagcaaaaaa

SEQ ID NO: 29 - DNA, 37 bp, synthetic construct
  misc_feature 1..37: note = Description of Artificial Sequence: Synthetic oligonucleotide
  misc_feature 1..37: note = Description of Combined DNA/RNA Molecule: Synthetic oligonucleotide
  source 1..37: mol_type = other DNA
gctagttattgctcaggcccgattgtcgttgaaatcc

SEQ ID NO: 30 - DNA, 45 bp, synthetic construct
  misc_feature 1..45: note = Description of Artificial Sequence: Synthetic oligonucleotide
  misc_feature 1..45: note = Description of Combined DNA/RNA Molecule: Synthetic oligonucleotide
  source 1..45: mol_type = other DNA
agagttgctaaaattgggcaaaagatcatcattagaagagttaca

SEQ ID NO: 31 - DNA, 26 bp, synthetic construct
  misc_feature 1..26: note = Description of Artificial Sequence: Synthetic oligonucleotide
  source 1..26: mol_type = other DNA
gagttgctaaaattgggcaaaagatc

SEQ ID NO: 32 - DNA, 25 bp, synthetic construct
  misc_feature 1..25: note = Description of Artificial Sequence: Synthetic oligonucleotide
  source 1..25: mol_type = other DNA
gcggatcatttatgctgagtttatc

SEQ ID NO: 33 - DNA, 60 bp, synthetic construct
  misc_feature 1..60: note = Description of Artificial Sequence: Synthetic oligonucleotide
  variation 34: note = any base
  source 1..60: mol_type = other DNA
acactctttccctacacgacgctcttccgatctngagttgctaaaattgggcaaaagatc

SEQ ID NO: 34 - DNA, 61 bp, synthetic construct
  misc_feature 1..61: note = Description of Artificial Sequence: Synthetic oligonucleotide
  variation 34..35: note = any base
  source 1..61: mol_type = other DNA
acactctttccctacacgacgctcttccgatctnngagttgctaaaattgggcaaaagatc

SEQ ID NO: 35 - DNA, 62 bp, synthetic construct
  misc_feature 1..62: note = Description of Artificial Sequence: Synthetic oligonucleotide
  variation 34..36: note = any base
  source 1..62: mol_type = other DNA
acactctttccctacacgacgctcttccgatctnnngagttgctaaaattgggcaaaagatc

SEQ ID NO: 36 - DNA, 63 bp, synthetic construct
  misc_feature 1..63: note = Description of Artificial Sequence: Synthetic oligonucleotide
  variation 34..37: note = any base
  source 1..63: mol_type = other DNA
acactctttccctacacgacgctcttccgatctnnnngagttgctaaaattgggcaaaagatc

SEQ ID NO: 37 - DNA, 64 bp, synthetic construct
  misc_feature 1..64: note = Description of Artificial Sequence: Synthetic oligonucleotide
  variation 34..38: note = any base
  source 1..64: mol_type = other DNA
acactctttccctacacgacgctcttccgatctnnnnngagttgctaaaattgggcaaaagatc

SEQ ID NO: 38 - DNA, 65 bp, synthetic construct
  misc_feature 1..65: note = Description of Artificial Sequence: Synthetic oligonucleotide
  variation 34..39: note = any base
  source 1..65: mol_type = other DNA
acactctttccctacacgacgctcttccgatctnnnnnngagttgctaaaattgggcaaaagatc

SEQ ID NO: 39 - DNA, 60 bp, synthetic construct
  misc_feature 1..60: note = Description of Artificial Sequence: Synthetic oligonucleotide
  variation 35: note = any base
  source 1..60: mol_type = other DNA
gtgactggagttcagacgtgtgctcttccgatctngcggatcatttatgctgagtttatc

SEQ ID NO: 40 - DNA, 61 bp, synthetic construct
  misc_feature 1..61: note = Description of Artificial Sequence: Synthetic oligonucleotide
  variation 35..36: note = any base
  source 1..61: mol_type = other DNA
gtgactggagttcagacgtgtgctcttccgatctnngcggatcatttatgctgagtttatc

SEQ ID NO: 41 - DNA, 62 bp, synthetic construct
  misc_feature 1..62: note = Description of Artificial Sequence: Synthetic oligonucleotide
  variation 35..37: note = any base
  source 1..62: mol_type = other DNA
gtgactggagttcagacgtgtgctcttccgatctnnngcggatcatttatgctgagtttatc

SEQ ID NO: 42 - DNA, 63 bp, synthetic construct
  misc_feature 1..63: note = Description of Artificial Sequence: Synthetic oligonucleotide
  variation 35..38: note = any base
  source 1..63: mol_type = other DNA
gtgactggagttcagacgtgtgctcttccgatctnnnngcggatcatttatgctgagtttatc

SEQ ID NO: 43 - DNA, 64 bp, synthetic construct
  misc_feature 1..64: note = Description of Artificial Sequence: Synthetic oligonucleotide
  variation 35..39: note = any base
  source 1..64: mol_type = other DNA
gtgactggagttcagacgtgtgctcttccgatctnnnnngcggatcatttatgctgagtttatc

SEQ ID NO: 44 - DNA, 65 bp, synthetic construct
  misc_feature 1..65: note = Description of Artificial Sequence: Synthetic oligonucleotide
  variation 35..40: note = any base
  source 1..65: mol_type = other DNA
gtgactggagttcagacgtgtgctcttccgatctnnnnnngcggatcatttatgctgagtttatc

SEQ ID NO: 45 - DNA, 59 bp, synthetic construct
  misc_feature 1..59: note = Description of Artificial Sequence: Synthetic oligonucleotide
  source 1..59: mol_type = other DNA
aatgatacggcgaccaccgagatctacacatagaggcacactctttccctacacgacgc

SEQ ID NO: 46 - DNA, 59 bp, synthetic construct
  misc_feature 1..59: note = Description of Artificial Sequence: Synthetic oligonucleotide
  source 1..59: mol_type = other DNA
aatgatacggcgaccaccgagatctacaccctatcctacactctttccctacacgacgc

SEQ ID NO: 47 - DNA, 53 bp, synthetic construct
  misc_feature 1..53: note = Description of Artificial Sequence: Synthetic oligonucleotide
  source 1..53: mol_type = other DNA
caagcagaagacggcatacgagattccggagagtgactggagttcagacgtgt

SEQ ID NO: 48 - DNA, 53 bp, synthetic construct
  misc_feature 1..53: note = Description of Artificial Sequence: Synthetic oligonucleotide
  source 1..53: mol_type = other DNA
caagcagaagacggcatacgagatcgctcattgtgactggagttcagacgtgt

SEQ ID NO: 49 - DNA, 39 bp, synthetic construct
  misc_feature 1..39: note = Description of Artificial Sequence: Synthetic oligonucleotide
  source 1..39: mol_type = other DNA
atgaaagtgaggaaatatattactttatgcttttggtgg

SEQ ID NO: 50 - DNA, 30 bp, synthetic construct
  misc_feature 1..30: note = Description of Artificial Sequence: Synthetic oligonucleotide
  source 1..30: mol_type = other DNA
gagaagcactgaaccccgtaacttaaggtg

SEQ ID NO: 51 - protein, 876 aa, Geobacillus stearothermophilus
MKKKLVLIDGNSVAYRAFFALPLLHNDKGIHTNAVYGFTMMLNKILAEEQPTHLLVAFDAGKTTFRHETFQEYKGGRQQTPPELSEQFPLLRELLKAYRIPAYELDHYEADDIIGTLAARAEQEGFEVKIISGDRDLTQLASRHVTVDITKKGITDIEPYTPETVREKYGLTPEQIVDLKGLMGDKSDNIPGVPGIGEKTAVKLLKQFGTVENVLASIDEVKGEKLKENLRQHRDLALLSKQLASICRDAPVELSLDDIVYEGQDREKVIALFKELGFQSFLEKMAAPAAEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSQIELRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

SEQ ID NO: 52 - protein, 704 aa, Escherichia phage T7
MIVSDIEANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGKRFGSHALEAWGYRLGEMKGEYKDDFKRMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQIAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICH

SEQ ID NO: 53 - protein, 898 aa, Enterobacteria phage T4
MKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 54 - protein, 832 aa, Thermus aquaticus [sequence omitted]

SEQ ID NO: 55 - protein, 1702 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSILPNEWLPIIENGEIKFVKIGEFINSYMEKQKENVKTVENTEVLEVNNLFAFSFNKKIKESEVKKVKALIRHKYKGKAYEIQLSSGRKINITAGHSLFTVRNGEIKEVSGDGIKEGDLIVAPKKIKLNEKGVSINIPELISDLSEEETADIVMTISAKGRKNFFKGMLRTLRWMFGEENRRIRTFNRYLFHLEKLGLIKLLPRGYEVTDWERLKKYKQLYEKLAGSVKYNGNKREYLVMFNEIKDFISYFPQKELEEWKIGTLNGFRTNCILKVDEDFGKLLGYYVSEGYAGAQKNKTGGISYSVKLYNEDPNVLESMKNVAEKFFGKVRVDRNCVSISKKMAYLVMKCLCGALAENKRIPSVILTSPEPVRWSFLEAYFTGDGDIHPSKRFRLSTKSELLANQLVFLLNSLGISSVKIGFDSGVYRVYINEDLQFPQTSREKNTYYSNLIPKEILRDVFGKEFQKNMTFKKFKELVDSGKLNREKAKLLEFFINGDIVLDRVKSVKEKDYEGYVYDLSVEDNENFLVGFGLLYAHNSYYGYMGYPKARWYSKECAESVTAWGRHYIEMTIREIEEKFGFKVLYADSVSGESEIIIRQNGKIRFVKIKDLFSKVDYSIGEKEYCILEGVEALTLDDDGKLVWKPVPYVMRHRANKRMFRIWLTNSWYIDVTEDHSLIGYLNTSKTKTAKKIGERLKEVKPFELGKAVKSLICPNAPLKDENTKTSEIAVKFWELVGLIVGDGNWGGDSRWAEYYLGLSTGKDAEEIKQKLLEPLKTYGVISNYYPKNEKGDFNILAKSLVKFMKRHFKDEKGRRKIPEFMYELPVTYIEAFLRGLFSADGTVTIRKGVPEIRLTNIDADFLREVRKLLWIVGISNSIFAETTPNRYNGVSTGTYSKHLRIKNKWRFAERIGFLIERKQKRLLEHLKSARVKRNTIDFGFDLVHVKKVEEIPYEGYVYDIEVEETHRFFANNILVHNTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 56 - protein, 605 aa, Escherichia coli
VISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 57 - protein, 575 aa, Bacillus phage phi29 [sequence omitted]

SEQ ID NO: 58 - protein, 799 aa, Homo sapiens
GFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 59 - protein, 5 aa, synthetic construct
  REGION 1..5: note = Description of Artificial Sequence: Synthetic peptide
  MOD_RES 3: note = Any amino acid
LPXTG

SEQ ID NO: 60 - protein, 21 aa, synthetic construct
  REGION 1..21: note = Description of Artificial Sequence: Synthetic peptide
  SITE 1..21: note = This sequence may encompass 1-7 "Gly Gly Gly" repeating units
GGGGGGGGGGGGGGGGGGGGG

SEQ ID NO: 61 - protein, 35 aa, synthetic construct
  REGION 1..35: note = Description of Artificial Sequence: Synthetic polypeptide
  SITE 1..35: note = This sequence may encompass 1-7 "Gly Gly Gly Gly Ser" repeating units
GGGGSGGGGSGGGGSGGGGSGGGGSGGGGSGGGGS

SEQ ID NO: 62 - protein, 28 aa, synthetic construct
  REGION 1..28: note = Description of Artificial Sequence: Synthetic peptide
  SITE 1..28: note = This sequence may encompass 1-7 "Gly Gly Gly Ser" repeating units
GGGSGGGSGGGSGGGSGGGSGGGSGGGS

SEQ ID NO: 63 - protein, 4 aa, synthetic construct
  REGION 1..4: note = Description of Artificial Sequence: Synthetic peptide
GGGS

SEQ ID NO: 64 - DNA, 23 bp, synthetic construct
  misc_feature 1..23: note = Description of Artificial Sequence: Synthetic oligonucleotide
  source 1..23: mol_type = other DNA
atcattagaagagttacaggggg

SEQ ID NO: 65 - DNA, 16 bp, synthetic construct
  misc_feature 1..16: note = Description of Artificial Sequence: Synthetic oligonucleotide
  source 1..16: mol_type = other DNA
gagttacaaagtggtg

SEQ ID NO: 66 - DNA, 461 bp, synthetic construct
  misc_feature 1..461: note = Description of Artificial Sequence: Synthetic polynucleotide
  source 1..461: mol_type = other DNA
agagttgctaaaattgggcaaaagatcatcattagaagagttacaaagtggtggagggagtatggtcagcaagggagcagagttgtttacaggaattgtgcctattttgatcgagcttaatggtgatgtcaatggtcacaagttcagcgtctctggagagggcgaaggggatgccacatacggtaagcttacgtttgttaccggtaaaagaagctgaggataaactcagcataaatgatccgctttttgagaggcagtggcacttggtcaatccaagttttcctggcagtgatataaatgttcttgatctgtggtacaataatattacaggcgcaggggtcgtggctgccattgttgatgatggccttgactacgaaaatgaagacttgaaggataatttttgcgctgaaggttcttgggatttcaacgacaatcgggcctgagcaataactagcaaaaaa

SEQ ID NO: 67 - protein, 4 aa, synthetic construct
  REGION 1..4: note = Description of Artificial Sequence: Synthetic peptide
GGGS

SEQ ID NO: 68 - DNA, 23 bp, synthetic construct
  misc_feature 1..23: note = Description of Artificial Sequence: Synthetic oligonucleotide
  source 1..23: mol_type = other DNA
tgtccagtgggcttttcagttcg

SEQ ID NO: 69 - DNA, 20 bp, synthetic construct
  misc_feature 1..20: note = Description of Artificial Sequence: Synthetic oligonucleotide
  source 1..20: mol_type = other DNA
gaaaagcccactggacagtc

SEQ ID NO: 70 - RNA, 100 nt, synthetic construct; identical to SEQ ID NO: 8
  misc_feature 1..100: note = Description of Artificial Sequence: Synthetic polynucleotide
  source 1..100: mol_type = other RNA

SEQ ID NO: 71 - RNA, 120 nt, synthetic construct; identical to SEQ ID NO: 9
  misc_feature 1..120: note = Description of Artificial Sequence: Synthetic polynucleotide
  source 1..120: mol_type = other RNA

SEQ ID NO: 72 - protein, 8 aa, synthetic construct
  REGION 1..8: note = Description of Artificial Sequence: Synthetic peptide
GGGSGGGS

SEQ ID NO: 73 - DNA, 140 bp, Saccharomyces cerevisiae
  source 1..140: mol_type = unassigned DNA
gagttgctaaaattgggcaaaagatcatcattagaagagttacagggggataacaacgaccacatattatctgtccatgatttattcccgcgtaacgacctatttaagagactaccggtgcctgctccaccaatggactc

SEQ ID NO: 74 - DNA, 134 bp, Saccharomyces cerevisiae
  source 1..134: mol_type = unassigned DNA
tgctaaaattgggcaaaagatcatcattagaagagttacaaagtggtggagggagtatggtcagcaagggagcagagttgtttacaggaattgtgcctattttgatcgagcttaatggtgatgtcaatggtcac

SEQ ID NO: 75 - DNA, 135 bp, Saccharomyces cerevisiae
  source 1..135: mol_type = unassigned DNA
gctaaaattgggcaaaagatcatcattagaagagttacagggggataacaacgaccacatattatctgtccatgatttattcccgcgtaacgacctatttaagagactaccggtgcctgctccaccaatggactc

SEQ ID NO: 76 - DNA, 136 bp, Saccharomyces cerevisiae
  source 1..136: mol_type = unassigned DNA
tgctaaaattgggcaaaagatcatcattagaagagttacagggggataacaacgaccacatattatctgtccatgatttattcccgcgtaacgacctatttaagagactaccggtgcctgctccaccaatggactc

SEQ ID NO: 77 - DNA, 139 bp, Saccharomyces cerevisiae
  source 1..139: mol_type = unassigned DNA
agttgctaaaattgggcaaaagatcatcattagaagagttacagggggataacaacgaccacatattatctgtccatgatttattcccgcgtaacgacctatttaagagactaccggtgcctgctccaccaatggactc

SEQ ID NO: 78 - DNA, 138 bp, Saccharomyces cerevisiae
  source 1..138: mol_type = unassigned DNA
gagttgctaaaattgggcaaaagatcatcattagaagagttacaaagtggtggagggagtatggtcagcaagggagcagagttgtttacaggaattgtgcctattttgatcgagcttaatggtgatgtcaatggtcac

SEQ ID NO: 79 - DNA, 139 bp, Saccharomyces cerevisiae
  source 1..139: mol_type = unassigned DNA
gagttgctaaaattgggcaaaagatcatcattagaagagttacagggggataacaacgaccacatattatctgtccatgatatattcccgcgtaacgacctatttaagagagtaccggtgcctgctccaccaatggact

SEQ ID NO: 80 - DNA, 137 bp, Saccharomyces cerevisiae
  source 1..137: mol_type = unassigned DNA
agttgctaaaattgggcaaaagatcatcattagaagagttacaaagtggtggagggagtatggtcagcaagggagcagagttgtttacaggaattgtgcctattttgatcgagcttaatggtgatgtcaatggtcac

SEQ ID NO: 81 - DNA, 138 bp, Saccharomyces cerevisiae
  source 1..138: mol_type = unassigned DNA
gttgctaaaattgggcaaaagatcatcattagaagagttacagggggataacaacgaccacatattatctgtccatgatttattcccgcgtaacgacctatttaagagactaccggtgcctgctccaccaatggactc

SEQ ID NO: 82 - DNA, 16 bp, Saccharomyces cerevisiae
  source 1..16: mol_type = unassigned DNA
gagttacaaagtggtg

(19) United States
(12) Patent Application Publication
(10) Pub. No.: US 2024/0101987 A1
(43) Pub. Date: Mar. 28, 2024

(54) POLYPEPTIDE FUSIONS OR CONJUGATES FOR GENE EDITING

(71) Applicant: 4M GENOMICS INC., Pleasanton, CA (US)

(72) Inventors: Arek BIBILLO, Walnut Creek, CA (US); Pranav PATEL, Pleasanton, CA (US)

(21) Appl. No.: 18/351,747

(22) Filed: Jul. 13, 2023

Related U.S. Application Data
(63) Continuation of application No. PCT/US2022/012616, filed on Jan. 14, 2022.
(60) Provisional application No. 63/138,289, filed on Jan. 15, 2021.

(51) Int. Cl. C12N 9/22; C12N 9/12; C12N 15/11; C12N 15/90

(52) U.S. Cl. CPC C12N 9/22; C12N 9/1252; C12N 15/11; C12N 15/111; C12N 15/905; C12Y 207/07007; C12N 2310/20

(57) ABSTRACT

Provided herein are improved methods, compositions, and systems for editing genomic DNA, including editing genomic DNA by inserting long DNA sequences into genomic DNA.

### CROSS-REFERENCE

This application is a continuation application of International Patent Application No. PCT/US2022/012616, filed Jan. 14, 2022, which claims the benefit of U.S. Provisional Application No. 63/138,289, filed on Jan. 15, 2021, each of which is incorporated in its entirety herein.

### SEQUENCE LISTING

The instant application contains a Sequence Listing which has been submitted electronically in XML format and is hereby incorporated by reference in its entirety. Said XML copy, created on Aug. 9, 2023, is named 59405-701_301_SL.xml and is 125,192 bytes in size.

### BACKGROUND

Programmable nucleases such as CRISPR-associated Cas endonucleases and TALEN endonucleases have revolutionized the ability to perform gene editing in organisms in a precise, site-directed manner.

### SUMMARY

In some aspects, the present disclosure provides for a composition comprising a fusion protein comprising: (a) a programmable nuclease configured to bind a double-stranded deoxyribonucleic acid (DNA) site; and (b) a polypeptide with DNA polymerase activity linked to the programmable nuclease. In some embodiments, the programmable nuclease is configured to cleave at least one strand of DNA at the double-stranded DNA site. In some embodiments, the programmable nuclease is a Cas protein or a Transcription activator-like effector nuclease (TALEN). In some embodiments, the programmable nuclease is a Cas protein, wherein the Cas protein is a Class 2, Type II Cas protein or a Class 2, Type V Cas protein. In some embodiments, the programmable nuclease comprises a Cas9 protein, a Cas12a protein, a Cas12b protein, a Cas12c protein, a Cas12d protein, a Cas12e protein, a Cas 12f protein, a C2C10 protein, a Cas14ab protein, a Type V-U1 protein, a Type V-U2 protein, a Type V-U3 protein, a Type V-U4 protein, a Type V-U5 protein, a derivative thereof, or a hybrid thereof. In some embodiments, the composition further comprises a guide polynucleotide configured to interact with the Cas protein, wherein the guide polynucleotide is configured to hybridize to the double-stranded DNA site. In some embodiments, the guide polynucleotide comprises DNA, ribonucleic acid (RNA), or a combination thereof. In some embodiments, the programmable nuclease is a TALEN, wherein the TALEN comprises at least one transcription activator-like effector (TAL) DNA-binding domain and an endonuclease domain. In some embodiments, the endonuclease domain comprises a FokI endonuclease domain or a PvuII endonuclease domain. In some embodiments, the composition further comprises an insert DNA molecule comprising a region with complementarity to a region 5′ to the double-stranded DNA site or a region with complementarity to a region 3′ to the nucleic acid site. In some embodiments, the region with complementarity to a region 5′ to the nucleic acid site or the region with complementarity to a region 3′ to the nucleic acid site comprises at least 4 to 30 bp or at least 4 to 400 bp. In some embodiments, the region with complementarity to a region 5′ to the nucleic acid site or a region with complementarity to a region 3′ to the nucleic acid site, wherein the region comprises a mismatch or mutation of at least 1 bp to at least 5 bp. In some embodiments, the programmable nuclease is a Cas endonuclease, wherein the Cas In some embodiments, the single endonuclease domain is a RuvC domain. In some embodiments, the insert nucleic acid sequence comprises at least about 1 bp to at least about 20 kb. In some embodiments, the insert DNA molecule is a single-stranded deoxyribonucleic acid molecule , at least partially a single-stranded deoxyribonucleic acid molecule, or at least partially a double-stranded deoxyribonucleic acid molecule. In some embodiments, the insert DNA molecule is a double-stranded deoxyribonucleic acid molecule. In some embodiments, the insert DNA molecule is at least partially a double-stranded deoxyribonucleic acid molecule, wherein the insert DNA molecule comprises a single-stranded region at a 3′ end and a single-stranded region at a 5′ end. In some embodiments, the insert DNA molecule is: (i) linked to the programmable nuclease; (ii) linked to a guide polynucleotide configured to interact with a Cas endonuclease, wherein the programmable nuclease is a Cas enzyme; or (iii) hybridized to a guide polynucleotide configured to interact with a Cas endonuclease, wherein the programmable nuclease is a Cas enzyme. In some embodiments, the programmable nuclease is a Cas protein, wherein the composition further comprises a guide polynucleotide configured to interact with the Cas protein, wherein the guide polynucleotide RNA further comprises a hybridization domain at a 3′ end; and wherein the insert DNA molecule comprises a first end configured to hybridize with the hybridization domain of the guide polynucleotide at the 3′ end of the insert DNA. In some embodiments, the insert DNA molecule comprises a region with complementarity to a region 5′ to the double-stranded DNA site at the 5′ end of the insert DNA. In some embodiments, the polypeptide with DNA polymerase activity is linked N-terminal to the programmable nuclease. In some embodiments, the polypeptide with DNA polymerase activity is linked C-terminal to the programmable nuclease. In some embodiments, the composition further comprises a linker between the programmable nuclease and the polypeptide with DNA polymerase activity. In some embodiments, the linker comprises a peptide bond, an isopeptide bond, a small molecule linker, or a combination thereof. In some embodiments, the linker comprises LPXTG (SEQ ID NO: 59), GGG, (GGG)ₙ (SEQ ID NO: 60), (GGGGS)ₙ (SEQ ID NO: 61), (GGGS)ₙ (SEQ ID NO: 62), N₁₋₇, a biotin-streptavidin pair or an analog of a biotin-streptavidin pair, or SpyTag/SpyCatcher sequences linked by an isopeptide bond. In some embodiments, the polypeptide with DNA polymerase activity comprises a T7 DNA polymerase, Bst polymerase or an analog thereof, T4 DNA polymerase, Taq polymerase, Vent polymerase, Q5 polymerase, Klenow fragment, DNA polymerase theta, or Phi29 polymerase. In some embodiments, the polypeptide with DNA polymerase activity comprises a sequence having at least 80%, at least 85%, at least 86%, at least 87%, at least 88%, at least 89%, at least 90%, at least 91%, at least 92%, at least 93%, at least 94%, at least 95%, at least 96%, at least 97%, or at least 99% sequence identity to any one of SEQ ID NOs: 16, 26, 51, 52, 53, 54, 55, 56, 57, or 58, or a variant thereof. In some embodiments, the Cas protein comprises a sequence having at least 80%, at least 85%, at least 86%, at least 87%, at least 88%, at least 89%, at least 90%, at least 91%, at least 92%, at least 93%, at least 94%, at least 95%, at least 96%, at least 97%, or. at least 99% sequence identity to any one of SEQ ID NOs: 14 or 15, or a variant thereof. In some embodiments, the fusion protein comprises a sequence having at least 80%, at least 85%, at least 86%, at least 87%, at least 88%, at least 89%, at least 90%, at least 91%, at least 92%, at least 93%, at least 94%, at least 95%, at least 96%, at least 97%, or at least 99% sequence identity to SEQ ID NO: 26 or a variant thereof.

In some aspects, the present disclosure provides for a system comprising any of the compositions described herein.

In some aspects, the present disclosure provides for a nucleic acid sequence encoding the fusion protein or composition of any of the claims described herein.

In some aspects, the present disclosure provides for a method of editing a double stranded DNA site in a cell, comprising introducing to the cell any of the compositions described herein.

In some aspects, the present disclosure provides for a method of editing a double-stranded DNA site in a cell, comprising introducing to the cell: (a) a fusion protein comprising: (i) a programmable nuclease configured to bind a double-stranded DNA site wherein the programmable nuclease is a Cas protein; and (ii) a polypeptide with DNA polymerase activity linked to the programmable nuclease; (b) a guide polynucleotide configured to interact with the Cas protein and configured to target the genomic locus; and (c) an insert DNA molecule comprising a region with complementarity to a region 5′ to the double-stranded DNA site or a region with complementarity to a region 3′ to the nucleic acid site. In some embodiments, the region with complementarity to a region 5′ to the nucleic acid site or the region with complementarity to a region 3′ to the nucleic acid site comprises at least 4 to 30 bp or at least 4 to 400 bp. In some embodiments, the region with complementarity to a region 5′ to the nucleic acid site or a region with complementarity to a region 3′ to the nucleic acid site, wherein the region comprises a mismatch or mutation of at least 1 bp to at least 5 bp. In some embodiments, the Cas protein is a Class 2, Type II Cas protein or a Class 2, Type V Cas protein. In some embodiments, the programmable nuclease comprises a Cas9 protein, a Cas12a protein, a Cas12b protein, a Cas12c protein, a Cas12d protein, a Cas12e protein, a Cas 12f protein, a C2C10 protein, a Cas14ab protein, a Type V-U1 protein, a Type V-U2 protein, a Type V-U3 protein, a Type V-U4 protein, a Type V-U5 protein, a derivative thereof, or a hybrid thereof. In some embodiments, the guide polynucleotide comprises DNA, RNA, or a combination thereof. In some embodiments, the insert DNA molecule is a single-stranded deoxyribonucleic acid molecule, at least partially a single-stranded deoxyribonucleic acid molecule, or at least partially a double-stranded deoxyribonucleic acid molecule. In some embodiments, the insert DNA molecule is a double-stranded deoxyribonucleic acid molecule. In some embodiments, the insert DNA molecule is at least partially a double-stranded deoxyribonucleic acid molecule, wherein the insert DNA molecule comprises a single-stranded region at a 3′ end and a single-stranded region at a 5′ end. In some embodiments: the guide polynucleotide further comprises a hybridization domain at a 3′ end; and the insert DNA molecule comprises a first end configured to hybridize with the hybridization domain of the guide polynucleotide at the 3′ end of the insert DNA. In some embodiments, the insert DNA molecule comprises a region with complementarity to a region 5′ to the double-stranded DNA site at the 5′ end of the insert DNA. In some embodiments, the polypeptide with DNA polymerase activity is linked N-terminal to the programmable nuclease. In some embodiments, the polypeptide with DNA polymerase activity is linked C-terminal to the programmable nuclease. In some embodiments, the fusion protein further comprises a linker between the programmable nuclease and the polypeptide with DNA polymerase activity. In some embodiments, the polypeptide with DNA polymerase activity comprises a T7 DNA polymerase, Bst polymerase or an analog thereof, T4 DNA polymerase, Taq polymerase, Vent polymerase, Q5 polymerase, Klenow fragment, DNA polymerase theta, or Phi29 polymerase. In some embodiments, the polypeptide with DNA polymerase activity comprises a sequence having at least 80%, at least 85%, at least 86%, at least 87%, at least 88%, at least 89%, at least 90%, at least 91%, at least 92%, at least 93%, at least 94%, at least 95%, at least 96%, at least 97%, or at least 99% sequence identity to any one of SEQ ID NOs: 16, 26, 51, 52, 53, 54, 55, 56, 57, or 58, or a variant thereof. In some embodiments, the Cas protein comprises a sequence having at least 80%, at least 85%, at least 86%, at least 87%, at least 88%, at least 89%, at least 90%, at least 91%, at least 92%, at least 93%, at least 94%, at least 95%, at least 96%, at least 97%, or. at least 99% sequence identity to any one of SEQ ID NOs: 14 or 15, or a variant thereof. In some embodiments, the method is at least about 3-times effective for introducing the DNA insert to the genomic locus, compared to the method using only a Cas protein without a polypeptide with DNA polymerase activity. In some embodiments, the method has at least about 10%, at least about 15%, at least about 20%, or at least about 25% efficiency for integration of the DNA insert to the genomic locus. In some embodiments, the cell is a prokaryotic cell. In some embodiments, the cell is a eukaryotic cell. In some embodiments, the cell is a yeast cell. In some embodiments, the cell is a human cell. In some embodiments, introducing to the cell further comprises contacting the cell with a nucleic acid or vector encoding the fusion protein or the guide polynucleotide. In some embodiments, introducing to the cell further comprises contacting the cell with a ribonucleoprotein complex (RNP) comprising the fusion protein or the guide polynucleotide.

In some aspects, the present disclosure provides for a vector comprising or any of the compositions described herein.

In some aspects, the present disclosure provides for a host cell comprising any of the vectors or nucleic acids described herein. In some embodiments, the cell is a prokaryotic cell. In some embodiments, the cell is a eukaryotic cell. In some embodiments, the cell is a yeast cell or a human cell.

In some aspects, the present disclosure provides for a composition comprising: (a) a programmable nuclease configured to bind a double-stranded DNA site; and (b) a polypeptide with DNA polymerase activity linked to the programmable nuclease. In some embodiments, the programmable nuclease is configured to cleave at least one strand of DNA at the double-stranded DNA site. In some embodiments, the programmable nuclease is a Cas protein or a Transcription activator-like effector nuclease (TALEN). In some embodiments, the programmable nuclease is a Cas protein, wherein the Cas protein is a Class 2, Type II Cas protein or a Class 2, Type V Cas protein. In some embodiments, the programmable nuclease comprises a Cas9 protein, a Cas12a protein, a Cas12b protein, a Cas12c protein, a Cas12d protein, a Cas12e protein, a Cas 12f protein, a C2C10 protein, a Cas14ab protein, a Type V-U1 protein, a Type V-U2 protein, a Type V-U3 protein, a Type V-U4 protein, a Type V-U5 protein, a derivative thereof, or a hybrid thereof. In some embodiments, the composition further comprises a guide polynucleotide configured to interact with the Cas protein, wherein the guide polynucleotide is configured to hybridize to the double-stranded DNA site. In some embodiments, the guide polynucleotide comprises DNA, RNA, or a combination thereof. In some embodiments, the programmable nuclease is a TALEN, wherein the TALEN comprises at least one Transcription activator-like effector (TAL) DNA-binding domain and an endonuclease domain. In some embodiments, the endonuclease domain comprises a FokI endonuclease domain or a PvuII endonuclease domain. In some embodiments, the composition further comprises an insert DNA molecule comprising a region with complementarity to a region 5′ to the double-stranded DNA site. In some embodiments, the region with complementarity to a region 5′ to the nucleic acid site or the region with complementarity to a region 3′ to the nucleic acid site comprises at least 4 to 30 bp or 4 to 400 bp. In some embodiments, the region with homology to a region 5′ to the nucleic acid site or a region with homology to a region 3′ to the nucleic acid site, wherein the region comprises a mismatch or mutation of at least 1 bp to 5 bp. In some embodiments, the programmable nuclease is a Cas endonuclease, wherein the Cas endonuclease comprises an inactivating mutation in a single endonuclease domain. In some embodiments, the single endonuclease domain is a RuvC domain In some embodiments, the insert nucleic acid sequence comprises 1 bp to 20 kb. In some embodiments, the insert DNA molecule is a single-stranded deoxyribonucleic acid molecule , at least partially a single-stranded deoxyribonucleic acid molecule, or at least partially a double-stranded deoxyribonucleic acid molecule. In some embodiments, the insert DNA molecule is: (i) linked to the programmable nuclease; (ii) linked to a guide polynucleotide configured to interact with a Cas endonuclease, wherein the programmable nuclease is a Cas enzyme; or (iii) hybridized to a guide polynucleotide configured to interact with a Cas endonuclease, wherein the programmable nuclease is a Cas enzyme. In some embodiments, the programmable nuclease is a Cas protein, wherein the composition further comprises a guide polynucleotide configured to interact with the Cas protein, wherein (a) the guide polynucleotide further comprises a hybridization domain at a 3′ end; and/or (b) wherein the insert DNA molecule comprises a first end configured to hybridize with the hybridization domain of the heterologous guide polynucleic acid. In some embodiments, the insert DNA molecule comprises the region with complementarity to a region 5′ to the double-stranded DNA site at a second end. In some embodiments, the polypeptide with DNA polymerase activity is linked N-terminal to the programmable nuclease. In some embodiments, the polypeptide with DNA polymerase activity is linked C-terminal to the programmable nuclease. In some embodiments, the programmable nuclease is linked to the polypeptide with DNA polymerase activity via a linker. In some embodiments, the linker comprises a peptide bond, an isopeptide bond, a small molecule linker, or a combination thereof. In some embodiments, the linker comprises LPXTG (SEQ ID NO: 59), GGG, (GGG)ₙ (SEQ ID NO: 60), (GGGGS)ₙ (SEQ ID NO: 61), (GGGS)ₙ (SEQ ID NO: 62), N₁₋₇, a biotin-streptavidin pair or an analog of a biotin-streptavidin pair, or SpyTag/SpyCatcher sequences linked by an isopeptide bond. In some embodiments, the polypeptide with DNA polymerase activity comprises a T7 DNA polymerase, Bst polymerase or an analog thereof, T4 DNA polymerase, Taq polymerase, Vent polymerase, Q5 polymerase, Klenow fragment, DNA polymerase theta, or Phi29 polymerase.

In some aspects, the present disclosure provides for a system comprising: (a) a class 2, type V Cas endonuclease capable of cleaving at least one strand of a DNA duplex; (b) a polypeptide with polymerase activity linked to the Cas endonuclease; (c) a guide polynucleotide comprising (i) a region targeting a DNA site in a cellular genome and (b) a region binding the class 2, type V Cas endonuclease, wherein the guide polynucleotide is configured to direct the class 2, type V cas endonuclease to cleave a at least one strand of DNA at a DNA site to generate a 3′ and a 5′ cleavage product; and/or (d) an insert DNA molecule comprising a 3′ arm capable of hybridizing with the 5′ cleavage product cleaved from the nucleic acid site in the cellular genome. In some embodiments, the class 2, type V Cas endonuclease comprises a Cas12a protein, a Cas12b protein, a Cas12c protein, a Cas12d protein, a Cas12e protein, a Cas12f protein, a C2C10 protein, a Cas14ab protein, a Type V-U1 protein, a Type V-U2 protein, a Type V-U3 protein, a Type V-U4 protein, a Type V-U5 protein, a derivative thereof, or a hybrid thereof. In some embodiments, the insert DNA molecule comprises an insert DNA sequence contiguous with the 3′ arm. In some embodiments, the 3′ arm comprise at least 4 to 400 base pairs. In some embodiments, the insert DNA sequence comprises at least 1 bp to 20 kb. In some embodiments, the insert DNA molecule is a single-stranded deoxyribonucleic acid molecule , at least partially a single-stranded deoxyribonucleic acid molecule, or at least partially a double-stranded deoxyribonucleic acid molecule. In some embodiments, the insert DNA molecule is: (i) covalently linked to the guide polynucleotide; or (ii) hybridized to the guide polynucleotide. In some embodiments of the system: (a) the guide polynucleotide further comprises a hybridization domain at a 3′ end; and/or (b) the insert DNA molecule comprises a first end configured to hybridize with the hybridization domain of the heterologous guide polynucleic acid. In some embodiments of the system: (a) the guide polynucleotide further comprises a hybridization domain at a 5′ end; and/or (b) the insert DNA molecule comprises a first end configured to hybridize with the hybridization domain of the heterologous guide polynucleic acid. In some embodiments, the class 2, type V Cas endonuclease is Cas12a. In some embodiments, the insert DNA molecule comprises the 5′ or 3′ arm at a second end. In some embodiments, the polypeptide with DNA polymerase activity is linked N-terminal to the programmable nuclease In some embodiments, the polypeptide with DNA polymerase activity is linked C-terminal to the programmable nuclease. In some embodiments, the system further comprises a linker between the programmable nuclease and the polypeptide with DNA polymerase activity. In some embodiments, the linker comprises a peptide bond, an isopeptide bond, a small molecule linker, or a combination thereof. In some embodiments, the linker comprises LPXTG (SEQ ID NO: 59), GGG, (GGG)ₙ (SEQ ID NO: 60), (GGGGS)ₙ (SEQ ID NO: 61), (GGGS)ₙ (SEQ ID NO: 62), N₁₋₇, a biotin-streptavidin pair or an analog of a biotin-streptavidin pair, or SpyTag/SpyCatcher sequences linked by an isopeptide bond. In some embodiments, the polypeptide with polymerase activity has DNA polymerase activity. In some embodiments, the polypeptide with DNA polymerase activity comprises a T7 DNA polymerase, Bst polymerase or an analog thereof, a T4 DNA polymerase, a Taq polymerase, a Vent polymerase, a Q5 polymerase, a Klenow fragment, a Phi29 polymerase, a functional fragment thereof, or a combination thereof.

In some aspects, the present disclosure provides for a composition comprising: (a) a programmable nuclease configured to bind a double-stranded DNA site; and/or (b) a polypeptide having DNA topoisomerase activity linked to the programmable nuclease, wherein the polypeptide having DNA topoisomerase activity contains a catalytic hydroxyl group linked to an insert DNA template. In some embodiments, the programmable nuclease is configured to cleave at least one strand of DNA at the double-stranded DNA site. In some embodiments, the programmable nuclease is a Cas protein or a Transcription activator-like effector nuclease (TALEN). In some embodiments, the programmable nuclease is a Cas protein, wherein the Cas protein is a Class 2, Type II Cas protein or a Class 2, Type V Cas protein. In some embodiments, the programmable nuclease comprises a Cas9 protein, a Cas12a protein, a Cas12b protein, a Cas12c protein, a Cas12d protein, a Cas12e protein, a Cas12f protein, a C2C10 protein, a Cas14ab protein, a Type V-U1 protein, a Type V-U2 protein, a Type V-U3 protein, a Type V-U4 protein, a Type V-U5 protein, a derivative thereof, or a hybrid thereof. In some embodiments, the composition further comprises a guide polynucleotide configured to interact with the Cas protein, wherein the guide polynucleotide is configured to hybridize to the nucleic acid site in the genome. In some embodiments, the guide polynucleotide comprises RNA or DNA. In some embodiments, the programmable nuclease is a TALEN, wherein the TALEN comprises at least one TAF effector DNA-binding domain and a FokI endonuclease domain. In some embodiments, the composition further comprises an insert DNA molecule comprising a region homologous to a region 5′ to the nucleic acid site or a region homologous to a region 3′ to the nucleic acid site contiguous with an insert nucleic acid sequence. In some embodiments, the region homologous to a region 5′ to the nucleic acid site or the region homologous to a region 3′ to the nucleic acid site comprises at least 4 base pairs to 400 base pairs. In some embodiments, the insert nucleic acid sequence comprises 1 base pair to 20kb. In some embodiments, the insert DNA molecule is a single-stranded deoxyribonucleic acid molecule , at least partially a single-stranded deoxyribonucleic acid molecule, or at least partially a double-stranded deoxyribonucleic acid molecule. In some embodiments, the insert DNA molecule is linked to the catalytic hydroxyl group of the polypeptide having DNA topoisomerase activity at a first end, and wherein the insert DNA molecule comprises the region homologous to a region 5′ to the nucleic acid site or the region homologous to a region 3′ to the nucleic acid site at a second end. In some embodiments, the polypeptide having DNA topoisomerase activity is linked N-terminal to the programmable nuclease. In some embodiments, the polypeptide having DNA topoisomerase activity is linked C-terminal to the programmable nuclease. In some embodiments, the composition further comprises a linker between the programmable nuclease and the polypeptide having DNA topoisomerase activity. In some embodiments, the linker comprises a peptide bond, an isopeptide bond, a small molecule linker, or a combination thereof. In some embodiments, the linker comprises LPXTG (SEQ ID NO: 59), GGG, (GGG)ₙ (SEQ ID NO: 60), (GGGGS)ₙ (SEQ ID NO: 61), (GGGS)ₙ (SEQ ID NO: 62), N₁₋₇, a biotin-streptavidin pair or an analog of a biotin-streptavidin pair, or SpyTag/SpyCatcher sequences linked by an isopeptide bond. In some embodiments, the polypeptide having DNA topoisomerase activity comprises a Type I topoisomerase or a Type II topoisomerase. In some embodiments, the Type I topoisomerase comprises a Type 1A topoisomerase. In some embodiments, the Type 1A topoisomerase comprises E. coli Eubacterial DNA topoisomerase I, E. coli Eubacterial DNA topoisomerase III, S. cerevisiae Yeast DNA topoisomerase IIII, H. sapiens DNA topoisomerase IIIα or IIIβ, S. acidocaldarius eubacterial and archaeal reverse DNA gyrase, or M. kandleri eubacterial reverse gyrase. In some embodiments, the composition comprises a Type I topoisomerase, and the Type I topoisomerase comprises a Type 1B topoisomerase. In some embodiments, the composition comprises a Type 1B topoisomerase, and the Type 1B topoisomerase comprises H. sapiens eukaryotic DNA topoisomerase I, Vaccinia poxvirus topoisomerase I, or M. kandleri hyperthermophilic eubacterial DNA topoisomerase V. In some embodiments, the composition comprises a Type II topoisomerase, and the Type II topoisomerase comprises a Type IIA topoisomerase. In some embodiments, the composition comprises a Type IIA topoisomerase, and the Type IIA topoisomerase comprises E. coli eubacterial DNA gyrase, E. coli eubacterial DNA topoisomerase IV, S. cerevisiae yeast DNA topoisomerase II, or H. sapiens mammalian DNA topoisomerase IIα or IIβ. In some embodiments, the composition comprises a Type II topoisomerase, and the Type II topoisomerase comprises a Type IIB topoisomerase. In some embodiments, the composition comprises a Type IIB topoisomerase, and the Type IIB topoisomerase comprises S. shibatae archaeal DNA topoisomerase VI.

In some aspects, the present disclosure provides for a composition comprising a complex having the following linked components: (a) a polynucleotide comprising a region homologous to a nucleic acid site in a cellular genome; (b) a displacement annealing domain comprising: (i) a polypeptide having RecA-like activity; and/or (ii) at least one polypeptide having RecN-like activity; and/or (c) a polypeptide with DNA polymerase activity. In some embodiments, the displacement annealing domain comprises from N- to C- terminus: at least one first polypeptide with RecA-like activity, an optional first linker, a polypeptide having RecN-like activity, an optional second linker, and at least one second polypeptide having RecA-like activity. In some embodiments, the at least one first polypeptide with RecA-like activity comprises at least two, at least three, at least four, or at least five polypeptides with Rec-A like activity. In some embodiments, the at least one second polypeptide with RecA-like activity comprises at least two, at least three, at least four, or at least five polypeptides with Rec-A like activity. In some embodiments, the complex comprises the following polypeptides from N- to C-terminus: a polypeptide with Rec A-like activity, a polypeptide with RecN-like activity, a polypeptide with RecA-like activity, and a polypeptide with DNA polymerase activity. In some embodiments, the polypeptide with RecA-like activity is RecA from E. coli or Rad54 from H. sapiens. In some embodiments, the polypeptide with RecN-like activity is RecN from E. coli or Rad51 from H. sapiens. In some embodiments, the polypeptide with DNA polymerase activity comprises a T7 DNA polymerase, Bst polymerase or an analog thereof, a T4 DNA polymerase, a Taq polymerase, a Vent polymerase, a Q5 polymerase, a Klenow fragment, a Phi29 polymerase, a functional fragment thereof, or any combination thereof. In some embodiments, the polynucleotide comprising the region homologous to the nucleic acid site in the cellular genome is linked to an N-terminus or C-terminus of the displacement annealing domain. In some embodiments, the polynucleotide comprising the region homologous to the nucleic acid site in the cellular genome is linked to an N-terminus or a C-terminus of the polypeptide with DNA polymerase activity. In some embodiments, the region homologous to a nucleic acid site in a cellular genome comprises at least 10, at least 20, at least 30, at least 40, or at least 50 base pairs. In some embodiments, the polynucleotide comprising a region homologous to a nucleic acid site in a cellular genome further comprises an insert nucleic acid sequence comprising at least about 1 bp to at least about 20 kb.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts a schematic of a polymerase-programmable nuclease fusion, and an illustration of how such a complex can function to insert DNA into a target DNA site. In this depiction (A) the programmable nuclease is a Cas endonuclease, such as a Cas9 endonuclease, and an insert DNA template is linked to the DNA polymerase-Cas9 complex via hybridization to an end of the sgRNA used to target the nuclease to the DNA site. Upon nuclease targeting to the site (B), a 3′ end of the insert DNA template is able to hybridize to a region on one side of a DNA strand that will later be cleaved by the programmable endonuclease. Upon cleavage of the DNA strand by the programmable endonuclease (C), a free 3′ end of the target DNA is liberated. Once liberated, the free 3′ end of the target DNA is able to be extended by the polymerase fused to the programmable nuclease (D). Subsequent DNA repair by the cell at the target DNA site functions to integrate the insert DNA at the target site.

FIG. 1B depicts a schematic of a polymerase-programmable nuclease fusion, and an illustration of how a complex can function to introduce a DNA mutation near a target site. In this depiction (A) the programmable nuclease is a Cas endonuclease, such as a Cas9 endonuclease, bearing an inactivating mutation in one of the endonuclease domains (e.g. the RuvC endonuclease domain) and a DNA bearing a mutation within a 3′ domain that is capable of hybridizing to the top DNA strand near the sgRNA cleavage site is linked to the Cas-polymerase complex via hybridization to an end of the sgRNA used to target the nuclease to the DNA site. The 3′ domain of the DNA is capable of hybridizing to the top DNA strand via D-loop formation by the Cas endonuclease in the vicinity of the sgRNA target site; however, because the RuvC (top) domain of the Cas endonuclease is disabled, this strand is not cut and is able to be extended. The DNA polymerase linked to the Cas enzyme (B) is then able to extend the end of the DNA bearing the mutation along the top strand of the target DNA to generate a long DNA molecule bearing the mutation. Subsequent DNA repair by the cell of the cleavage site in the bottom strand (produced by the one functional domain of the endonuclease) integrates the strand bearing the mutation at the target DNA site in the genome.

FIG. 1C depicts one proposed arrangement of the hybridization site from the insert DNA, the cut site of the target DNA, the insert template, the hybridization site between the sgRNA and the insert template, and the hybridization of the “seed” region of the sgRNA to the target DNA for use with a polymerase-endonuclease fusion according to some of the methods according to the disclosure. In this arrangement, the sequence complementary to the target DNA site incorporated into the DNA insert template is included on the 3′ end of one strand of a double-stranded insert template, and is complementary to about 23 nucleotides immediately preceding the DNA cleavage site by the endonuclease or Cas9 on the top (5′-3′ oriented) DNA strand near the cleavage site. Figure discloses SEQ ID NOS 68-69, respectively, in order of appearance.

FIG. 2 depicts two example guide sgRNAs functional with a Cas endonuclease (e.g., Cas9) targeting a same DNA site for use in methods described herein, one without a hybridization arm for attachment of an insert DNA template (left) and with a hybridization arm for attachment of an insert DNA template (right). Figure discloses SEQ ID NOS 70-71, respectively, in order of appearance.

FIG. 3 depicts the experiment of Example 2, demonstrating that addition of a hybridization arm to an sgRNA does not impair DNA cleavage. The left panel depicts sequences of the GE-8 (without hybridization arm) and GE-9 (with hybridization arm). The right panel depicts an agarose gel of an experiment where GE-8 and GE-9 were both used with Cas9 to digest a target DNA site in lambda DNA. Figure discloses SEQ ID NOS 8-9, respectively, in order of appearance.

FIG. 4 depicts the QPCR results of the experiment of Example 3, demonstrating that Cas9 cleavage is able to generate a 3′ end that can be extended by a properly designed insert DNA template in a single reaction. Shown is a fluorescence vs cycle (Ct) plot for quantitation of target-insert DNA fusions generated in: (a) a reaction containing insert DNA, target, Cas9, polymerase, and sgRNA (leftmost curve); and (b) control reactions lacking one of the components (right 4 curves, labeled with missing component).

FIG. 5 depicts the results of the experiment of Example 4 or 4A, demonstrating that the combined cleavage/extension reaction of Example 3 accommodates a wide range of insert DNA sizes. Shown is an agarose gel of a check PCR reaction performed on combined cleavage/extension reactions incorporating progressively larger insert DNA templates (50 bp, 200 bp, 500 bp, 2000 bp).

FIG. 6 depicts the results of the experiment of Example 5, demonstrating that annealing the insert DNA template to the sgRNA such that it is colocalized with the Cas complex improves the efficiency of the combined cleavage/extension reaction. Shown is a fluorescence vs cycle (Ct) plot for quantitation of target-insert DNA fusions generated in: (1) a reaction where insert DNA was annealed to a hybridization arm of an sgRNA (leftmost curve); and (2) a reaction containing a comparable sgRNA lacking a hybridization arm (rightmost curve).

FIGS. 7, 8, 9 and 10 depict domain diagrams illustrating various different schemes for how Cas protein-DNA polymerase fusions can be generated from separately prepared: (a) Type II or Type V-A enzymes and (b) DNA polymerase, using the SpyTag/SpyCatcher system to link two separately translated peptides. Either the SpyTag or SpyCatcher can be on either end of the Cas enzyme as long as the other of the SpyTag or SpyCatcher is on either end of the DNA polymerase. Other complementary linking systems (e.g. biotin-streptavidin) systems can be used in place of the SpyTag/SpyCatcher system. These schemes also contemplate an optional a linker amino acid sequence which may be variable length and composition optimized to maximize performance of both CRISPR effector protein and DNA polymerase.

FIG. 11 depicts domain diagrams illustrating different schemes for how Cas protein-DNA polymerase fusions can be generated from cotranslated: (a) Type II or Type V-A enzymes and (b) DNA polymerase. DNA polymerase can be located on either the N-terminus or C-terminus of the Cas enzyme.

FIG. 12 depicts domain diagrams for different Cas members of the Class I Cas enzyme family (Type II, Type V-A, Type V-B, Type V-C, Type V-U1/U2/U5, and Type V-U4/U3). Contemplated within this disclosure are hybrids of any of the types of enzymes depicted in this figure, where either whole domains or portions thereof that are the same type between different classes depicted here are swapped between the classes. Also contemplated are hybrids where individual residues within a given domain are swapped for equivalent residues in the same domain from a different Type Cas enzyme.

FIGS. 13A, 13B, and 13C depict schematics of a topoisomerase-programmable nuclease fusions, and an illustration of how such a fusions can function to insert DNA into a target DNA site. In this depiction (A) the programmable nuclease is a Cas endonuclease (e.g. Cas9) and an insert DNA: (a) has one strand of one end covalently linked to a catalytic hydroxyl of the topoisomerase domain provided as part of the Cas9-topoisomerase fusion; and (b) has a region with homology to a region proximal to the target DNA cleavage site at the end opposite that linked to the topoisomerase. Upon nuclease targeting to the site (B) the top DNA strand is cleaved by the Cas enzyme to liberate a free hydroxyl group, which can act to displace the catalytic hydroxyl linked to the insert DNA. Displacement of the catalytic hydroxyl linked to the insert DNA template (C) results in linkage of the insert DNA to the target DNA via the liberated hydroxyl. Subsequent DNA repair mechanisms inside the cell result in integration of the insert DNA at the target site. Shown are depictions of this scheme for Topoisomerase type I (13B) and Topoisomerase type II(13A).

FIG. 14 depicts domain diagrams illustrating different schemes for how Cas protein-DNA polymerase fusions can be generated from cotranslated: (a) Type II or Type V-A enzymes and (b) topoisomerase. Topoisomerase can be located on either the N-terminus or C-terminus of the Cas enzyme.

FIGS. 15, 16, and 17 depict how oligonucleotides linked to a displacement annealing domain-DNA polymerase fusion can be used to introduce mutations at a target site in genomic DNA. The displacement annealing domains are shown in FIGS. 16 and 17 (top), and can comprise RecA and RecN domains, or their human counterparts (Rad51 and Rad54 domains). The displacement annealing domains can be connected to either the N-terminus or C-terminus of a polypeptide having DNA polymerase (FIGS. 16 and 17, bottom). An oligonucleotide which bears a mutation within a domain capable of hybridizing to a target DNA site is linked to a displacement annealing domain, which is in turn linked to the DNA polymerase (FIG. 15, A). The displacement annealing domain allows for hybridization of the oligonucleotide bearing the mutation to the target DNA (B). Once hybridized to the target DNA, the DNA polymerase linked to the displacement annealing domain allows for extension of the oligonucleotide bearing the mutation along the DNA strand it is hybridized to, generating a long template bearing the mutation which is later incorporated into genomic DNA by endogenous repair mechanism.

FIG. 18 depicts a schematic illustrating the arrangement of the system used for genome editing (particularly the insertion of a DNA insert at a specific locus), such as the genome editing performed in Example 7. The system comprises a fusion protein, a gRNA, and a DNA insert. The fusion protein comprises an endonuclease (e.g. a Cas effector) fused to a DNA polymerase (which can have stand displacement, high processivity & high-fidelity properties). The gRNA targets the desired insertion site in genomic DNA, is capable of binding to the Cas effector, and has an extended 3′ arm for DNA insert. The 3′ arm of the gRNA allows for hybridization to a DNA insert, which can range in size from e.g. about 50 nucleotides to about 5000 nucleotides in length. The DNA insert additionally comprises a 3′ single-stranded region capable of hybridizing to one of the DNA strands at the site targeted by the gRNA. Once the endonuclease specifically recognizes and cleaves the site targeted by the gRNA and cleaves the target DNA, the resulting 3′ end liberated from the DNA can hybridize to the insert DNA, which can then be extended by the DNA polymerase. The resulting product extended by the DNA polymerase is covalently attached to the target DNA on one end and has dsDNA flap. Without wishing to be bound by theory, it is understood that homologous recombination on the dsDNA flap side of the insert can allow integration of the whole DNA insert in a precise and efficient manner.

FIG. 19 depicts a schematic illustrating the design of a DNA insert used for genome editing according to the methods described herein, such as in Example 7. The insert template is a dsDNA with two 3′-single-stranded overhangs. One of the overhangs is configured to hybridize to gRNA adapter sequence. The second overhang is configured to anneal to the genomic target sequence near the Cas endonuclease cleavage site. After annealing of the overhang to the released by Cas endonuclease target 3′-end, the target DNA serves as an extension primer to produce an extended product by the DNA polymerase part of the endonuclease-DNA polymerase fusion.

FIG. 20 depicts a schematic illustrating insertion of a DNA insert according to the methods described herein, in this case targeting Kex2 as described in Example 7. The DNA insert (“390 DNA insert”, bottom) is 455 nucleotides in length. 3′ and 5′ regions of the DNA insert are homologous to regions of the Kex2 gene (“Wild-type Kex2 fragment in Yeast”, top) separated by a region of variable length (in this case, 95 nucleotides) that is to be deleted when the DNA insert is integrated. Between the Kex2 homology arms, the DNA insert comprises a GGGS linker (SEQ ID NO: 63) in-frame with a GFP sequence. Successful insertion of the DNA insert results in deletion of 95 nucleotides of the original Kex2 sequence. “Rank 1” in the figure illustrates the target sequence of 5′-ATCATTAGAAGAGTTACAGGGGG-3′ (SEQ ID NO: 64) targeted by the gRNA used in Example 7. Figure discloses SEQ ID NO: 72.

FIG. 21 illustrates a plasmid (pGE-112) used as a control (“Cas only method”) for genome editing experiments in yeast such as in Example 7. The plasmid comprises: (a) SpCas9 alone bearing an NLS signal without a fusion partner under an ScPGK1 promoter and ScPGK1 terminator, and (b) a gRNA designed to target Kex2 (“Kex2 sgRNA (rank 1)”) having a scaffold sequence binding to SpCas9 under control of a tRNA Phe promoter .

FIG. 22 illustrates a plasmid (pGE-113) used to test the efficiency of the Cas-DNA polymerase fusion method described herein for genome editing experiments in yeast such as in Example 7. The plasmid comprises: (a) SpCas9 alone fused to Bst polymerase via a linker bearing an NLS signal under an ScPGK1 promoter and ScPGK1 terminator, and (b) a gRNA designed to target Kex2 (“Kex2 sgRNA (rank 1)”) having a scaffold sequence binding to SpCas9 under control of a tRNA Phe promoter .

FIG. 23 summarizes an Illumina NGS analysis of the Kex2 editing experiment performed in Example 7, illustrating that the Cas-polymerase fusion method (“4M method”) successfully edits at the Kex2 site. The top sequence in the figure is the wild-type Kex2 sequence, while the bottom sequences are exemplary sequencing results of the genome editing condition using the Cas-polymerase fusion enzyme (“4M method”, the condition where yeast competent cells (EBY100) were electroporated with pGE113 and 390 insert DNA). The sequences shown are the vicinity of the Kex2-insert junction. Two type of sequences are observed: wild-type unrecombined sequences (sequences with fully highlighted residues) and chimeric recombined sequences including the 390 insert (sequences with highlighted and unhighlighted residues). The bar labeled “Rank 1” illustrates the gRNA targeting site (5′- ATCATTAGAAGAGTTACAGGGGG-3′ (SEQ ID NO: 64)). The Kex2-390 Insert junction (labeled in figure) shows precise insertion as predicted with no variability as illustrated by the characteristic 5′-GAGTTACA/AAGTGGTG-3′ (SEQ ID NO: 65) sequence. The results in this figure were generated using the NGS target library as described in Example 7. Figure discloses SEQ ID NOS 73-77, 74, 76-77, 74-75, 77, 75, 78, 74, 79, 74, 80, 75, and 81-82, respectively, in order of appearance.

FIG. 24 illustrates editing efficiency as assessed as Example 7 and compared between: (a) the “Cas only method” using electroporation of the pGE-112 plasmid into yeast, and (b) the “4M method” involving the Cas-DNApol fusion using electroporation of the pGE-113 plasmid into yeast. The left panel chart summarizes the two conditions assessed in this experiment, while the right panel graph illustrates efficiency of insertion of the DNA insert (% recombined sequence) by each method. As can be seen by the graph, the efficiency of DNA insertion is improved in the “4M method” approximately 3-fold over the “Cas only” method, indicating that the Cas-DNA polymerase fusion improves the fidelity of insertion of DNA. For the efficiency graph, efficiency was estimated using 483288 sequences for the “Cas only method” and 341994 sequences for the “4M method”.

FIG. 25 illustrates a yeast editing experiment performed as in Example 7 verifying the dependency of the editing reaction on the DNA-editing enzymes, demonstrating that the editing reaction requires the Cas-DNApol fusion and does not proceed with the insert alone. The left panel illustrates the various “leave one out” test conditions performed in this experiment, whereas the right panel illustrates PCR products generated from electroporated yeast for each test condition (amplified using one primer complementary to Kex2 sequence--GE-249; and a second primer complementary to the 347 DNA insert- GE-173). The product corresponding to the Kex2-DNA insert junction only appears in condition 3 (see arrow), demonstrating that both Cas-DNApol fusion (provided in pGE-113) and DNA insert are required for the recombination reaction in yeast cells.

FIG. 26 illustrates a yeast editing experiment performed as in Example 7 illustrating the effect of DNA insert concentration on genomic insertion efficiency compared between the “Cas only method” and the “4M method”. The left panel chart illustrates the various conditions assessed in this experiment (amounts of DNA are expressed in micrograms), while the right panel illustrates PCR products generated from electroporated yeast for each condition (amplified using one primer complementary to Kex2 sequence--GE-249; and a second primer complementary to the 347 DNA insert- GE-173). As can be seen by comparison of lanes A-C in the right panel (“Cas only method”) to lanes D-F (“4M method”), the “4M method” is markedly less dependent on insert DNA concentration, as recombination still occurs at the 1.2 μg and 0.3 μg insert conditions for the “4M method”, but does not occur at the 1.2 μg and 0.3 μg insert conditions for the “Cas only method”.

FIG. 27 illustrates a qPCR analysis of the same conditions tested in FIG. 26 to assess the recombination efficiency of each method more accurately at the different insert DNA concentrations. Shown are qPCR traces of Ct (cycle time) versus fluorescence compared between the two different methods (labeled as pGE113 for the “4M method” and pGE112 for the “Cas only method”) for each different DNA insert concentration assessed in FIG. 26. The difference between the methods was assessed as ˜2 Ct at 5 μg insert DNA, ˜15 Ct at 1.2 μg insert DNA, and ˜10 Ct at 0.3 μg insert DNA.

### DETAILED DESCRIPTION

There is a need for endonuclease compositions, methods, and systems that improve the efficiency of transgene insertions into precise locations for genomic editing. Insertion efficiencies of transgenes using CRISPR-Cas relying on simple homologous recombination can be in the single digits at for large inserts, making approaches relying on such methods technically laborious. Provided herein are methods, compositions, and systems for improved gene editing, particularly involving large insert DNAs.

### Definitions

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (RI. Freshney, ed. (2010)) (which are entirely incorporated by reference herein).

As used herein, the term “programmable nuclease” generally refers to endonucleases that are “targeted” (“programed”) to recognize and edit a pre-determined site in a genome of an organism. In an embodiment, the programmable nuclease can induce site specific DNA cleavage at a pre-determined site in a genome. In an embodiment, the programmable nuclease may be programmed to recognize a genomic location with a DNA binding protein domain, or combination of DNA binding protein domains.

As used herein, a “guide nucleic acid” or “guide polynucleotide” generally refers to a nucleic acid that may hybridize to another nucleic acid. A guide nucleic acid may be RNA. A guide nucleic acid may be DNA. The guide nucleic acid may be programmed to bind specifically to a nucleic acid with a particular sequence. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called a noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. Guide nucleic acids may comprise a nucleic acid targeting segment (e.g. a crRNA) and a protein binding sequence. Guide nucleic acids may comprise a nucleic acid targeting segment (e.g. a crRNA) a protein binding sequence, and a trans-activating RNA (e.g. a tracrRNA).

A guide nucleic acid may comprise a segment that can be referred to as a “nucleic acid-targeting segment” a “nucleic acid-targeting sequence” or a “seed sequence”. In some cases, the sequence is 19-21 nucleotides in length. In some cases, “nucleic acid-targeting segment” or a “nucleic acid-targeting sequence” comprises a crRNA. A nucleic acid-targeting segment may comprise a sub-segment that may be referred to as a “protein binding segment” or “protein binding sequence” or “Cas protein binding segment”.

The term “tracrRNA” or “tracr sequence”, as used herein, can generally refer to a nucleic acid with at least about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes, S. aureus, etc). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence. tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA sequence over a stretch of at least 6 contiguous nucleotides.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with parameters of ; the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

The term “optimally aligned” in the context of two or more nucleic acids or polypeptide sequences, generally refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

Included in the current disclosure are variants of any of the enzymes, proteins, or domains described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to any one of the endonuclease protein sequences described herein. In some embodiments, such conservatively substituted variants are functional variants. Such functional variants can encompass sequences with substitutions such that the activity of one or more critical active site residues or guide polynucleotide binding residues of the endonuclease are not disrupted.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues. In some embodiments, any of the endonucleases described herein can comprise a nickase mutation. In some embodiments, any of the endonucleases described herein can comprise a RuvC domain lacking nuclease activity. In some embodiments, any of the endonucleases described herein can be configured to cleave one strand of a double-stranded target deoxyribonucleic acid. In some embodiments, any of the endonucleases described herein can comprise can be configured to lack endonuclease activity or be catalytically dead.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

### Endonuclease Compositions

In some aspects, the present disclosure provides for a composition comprising a programmable nuclease configured to bind a double-stranded DNA site. The programmable nuclease can be linked to a polypeptide having a second enzymatic activity. The programmable nuclease can be fused to a polypeptide having a second enzymatic activity. The programmable nuclease can be conjugated to a polypeptide having a second enzymatic activity. The programmable nuclease can be configured to cleave at least one strand of DNA at the double-stranded DNA site. The programmable nuclease can be configured to cleave both strands of DNA at the double-stranded DNA site. The programmable nuclease can comprise a Cas protein or a Transcription activator-like effector nuclease (TALEN). Cas proteins suitable for the methods described herein include, but are not limited to, Class 2, Type II Cas proteins and a Class 2, Type V Cas proteins, including e.g., Cas9 proteins, Cas12a proteins, Cas12b proteins, Cas12c proteins, Cas12d proteins, Cas12e proteins, Cas 12f proteins, C2C10 proteins, Cas14ab proteins, Type V-U1 proteins, Type V-U2 proteins, Type V-U3 proteins, Type V-U4 proteins, Type V-U5 proteins, derivatives thereof, or hybrids thereof. The programmable nuclease can comprise a Transcription activator-like effector nuclease (TALEN). The Cas protein can comprise an inactivating mutation in one or both endonuclease domains. The Cas protein can comprise an inactivating mutation in a RuvC domain, an HNH domain, or both RuvC and HNH domains. The TALEN can comprise at least one, at least two, at least three, at least four, at least five, at least six, at least seven, at least 8, at least 9, or at least 10 Transcription activator-like effector (TAL) DNA-binding domains fused to an endonuclease domain. The endonuclease domain can comprise an endonuclease specific for four or fewer, three or fewer, two or fewer, one or fewer, or no nucleotide residues. The endonuclease domain can comprise a FokI endonuclease domain or a PvuII endonuclease domain.

When the programmable nuclease is a Cas enzyme, the composition comprising the programmable nuclease can further comprise a guide polynucleotide configured to interact with the Cas protein, wherein the guide polynucleotide is configured to hybridize to the double-stranded DNA site. The guide polynucleotide can comprise DNA, RNA, or a combination thereof. The guide nucleic acid can comprise a nucleic acid-targeting sequence and a Cas-binding sequence. The nucleic acid targeting sequence can comprise at least about 19-21 nucleotides in length that are configured to hybridize to the double-stranded DNA site. In some cases, the guide polynucleotide can comprise the nucleic acid-targeting sequence at a first end and a second end comprising a hybridization domain capable of hybridizing to at least one strand of an insert DNA molecule. In some cases, the hybridization domain is at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 nucleotides.

In some cases, the composition comprising the programmable nuclease configured to bind the double-stranded DNA site linked to the polypeptide having a second enzymatic activity further comprises an insert DNA molecule. The insert DNA molecule can comprise a region configured to hybridize to a region 5′ to the double-stranded DNA site. The insert DNA molecule can comprise a region with complementarity to a region 5′ to the double-stranded DNA site. The region configured to hybridize to the region 5′ to the double-stranded DNA site or the region with complementarity to a region 5′ to the double-stranded DNA site can comprise at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35 nucleotides, at least 50 nucleotides, at least 100 nucleotides, at least 150 nucleotides, at least 200 nucleotides, at least 250 nucleotides, at least 300 nucleotides, at least 350 nucleotides, or at least 400 nucleotides. In some embodiments, the region configured to hybridize to the region 5′ to the double-stranded DNA site or the region with complementarity to a region 5′ to the double-stranded DNA site comprises a mismatch of at least 1, at least 2, at least 3, at least 4, at least 5, or at least 6 nucleotides. In some cases when the insert DNA comprises a mismatch, the Cas protein can comprise an inactivating mutation in one or both endonuclease domains. In some cases when the insert DNA comprises a mismatch, the Cas protein can comprise an inactivating mutation in a RuvC domain. In some cases when the insert DNA comprises a mismatch, the region configured to hybridize or the region with complementarity to the region 5′ to the double-stranded DNA site comprises at least 10, 20, 30, 40, or 50 nucleotides between a hybridization domain to the guide polynucleotide and the region hybridizing 5′ to the double-stranded DNA site. The insert DNA molecule can comprise at least 1 nucleotide, at least 50 bp, at least 100 bp, at least 150 bp, at least 200 bp, at least 250 bp, at least 300 bp, at least 350 bp, at least 400 bp, at least 450 bp, at least 500 bp, at least 600 bp, at least 700 bp, at least 800 bp, at least 900 bp, at least 1000 bp, at least 1200 bp, at least 1400 bp, at least 1600 bp, at least 1800 bp, at least 2000 bp, at least 2500 bp, at least 3000 bp, at least 3500 bp, at least 4000 bp, at least 4500 bp, at least 5000 bp, at least 5500 bp, at least 6000 bp, at least 6500 bp, at least 7000 bp, at least 7500 bp, at least 8000 bp, at least 8500 bp, at least 9000 bp, at least 9500 bp, or at least 10,000 bp. The insert DNA molecule can comprise at least 1 nucleotide, at least 50 bp, at least 100 bp, at least 150 bp, at least 200 bp, at least 250 bp, at least 300 bp, at least 350 bp, at least 400 bp, at least 450 bp, at least 500 bp, at least 600 bp, at least 700 bp, at least 800 bp, at least 900 bp, at least 1000 bp, at least 1200 bp, at least 1400 bp, at least 1600 bp, at least 1800 bp, at least 2000 bp, at least 2500 bp, at least 3000 bp, at least 3500 bp, at least 4000 bp, at least 4500 bp, at least 5000 bp, at least 5500 bp, at least 6000 bp, at least 6500 bp, at least 7000 bp, at least 7500 bp, at least 8000 bp, at least 8500 bp, at least 9000 bp, at least 9500 bp, or at least 10,000 bp aside from the region complementary or the region configured to hybridize. The insert DNA molecule can be single stranded deoxyribonucleic acid molecule, at least partially a single-stranded deoxyribonucleic acid molecule, or at least partially a double-stranded deoxyribonucleic acid molecule. The insert DNA molecule can be linked to the programmable nuclease. The insert DNA molecule can comprise a hybridization domain configured to hybridize to a region of a guide polynucleotide. In some cases, the hybridization domain can comprise at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 nucleotides. The insert DNA molecule can be linked to a guide polynucleotide configured to interact with a Cas endonuclease, wherein the programmable nuclease is a Cas enzyme. The insert DNA molecule can be hybridized to a guide polynucleotide configured to interact with a Cas endonuclease, wherein the programmable nuclease is a Cas enzyme.

In some cases, the composition enables an improved efficiency of insertion of the insert DNA molecule. In some cases, the composition allows for at least about a 1%, 2%, 5%, 10%, 15%, 20%, 25%, 30%, 35%, 40%, 45%, or 50% efficiency of insertion of said insert DNA into a cell.

The programmable nuclease can be linked to a polypeptide having a second enzymatic activity, which can be a polymerase activity. The second enzymatic activity can comprise a DNA polymerase activity. Accordingly, the polypeptide with a second enzymatic activity can comprise a DNA polymerase or a functional fragment thereof. DNA polymerases suitable for use with the methods and compositions described herein include, but are not limited to, T7 DNA polymerase, Bst polymerase or analogs thereof, a T4 DNA polymerase, Taq polymerase, Vent polymerase, Q5 polymerase, Klenow fragment, Phi29 polymerase, functional fragments thereof, or combinations thereof. The DNA polymerase can be an isothermal DNA polymerase, such as Bst polymerase or Bst2.0 polymerase. The polypeptide with a second enzymatic activity can be linked N-terminal to the programmable nuclease. The polypeptide with a second enzymatic activity can be linked C-terminal to the programmable nuclease. The polypeptide with a second enzymatic activity can be linked to the programmable nuclease using a linker between the polypeptide with a second enzymatic activity and the programmable nuclease. The linker can comprise a peptide bond, an isopeptide bond, a small molecule linker, or a combination thereof. The linker can comprise LPXTG (SEQ ID NO: 59), (GGG)ₙ (SEQ ID NO: 60), (GGGGS)ₙ (SEQ ID NO: 61), (GGGS)ₙ (SEQ ID NO: 62), N₁₋₇, a biotin-streptavidin pair or an analog of a biotin-streptavidin pair, or SpyTag/SpyCatcher sequences linked by an isopeptide bond.

In some aspects, the present disclosure provides for a system comprising a class 2, type V Cas endonuclease capable of cleaving at least one strand of a DNA duplex and a polypeptide with polymerase activity linked to the Cas endonuclease. The system can further comprise a guide polynucleotide comprising (i) a region targeting a nucleic acid site in a cellular genome and (b) a region binding the class 2, type V Cas endonuclease, wherein the guide polynucleotide is configured to direct the class 2, type V cas endonuclease to cleave a at least one strand of DNA at a DNA site to generate a 3′ and a 5′ cleavage product. The system can further comprise an insert DNA molecule comprising a 3′ arm capable of hybridizing with the 5′ cleavage product cleaved from the nucleic acid site in the cellular genome.

The guide polynucleotide can have a variety of configurations. The guide polynucleotide can comprise DNA, RNA, or a combination thereof. The guide nucleic acid can comprise a nucleic acid-targeting sequence and a Cas-binding sequence. The nucleic acid targeting sequence can comprise at least about 19-21 nucleotides in length that are configured to hybridize to the double-stranded DNA site. In some cases, the guide polynucleotide can comprise the nucleic acid-targeting sequence at a first end and a second end comprising a hybridization domain capable of hybridizing to at least one strand of an insert DNA molecule. In some cases, the hybridization domain is at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 nucleotides.

The class 2, type V Cas endonuclease can comprise a variety of Cas proteins. Cas proteins suitable for the methods described herein include, but are not limited to, e.g., Cas12a proteins, Cas12b proteins, Cas12c proteins, Cas12d proteins, Cas12e proteins, Cas 12f proteins, C2C10 proteins, derivatives thereof, or hybrids thereof. The Cas protein can comprise an inactivating mutation in one or both endonuclease domains.

The system can further comprise an insert DNA molecule comprising a 3′ arm capable of hybridizing with the 5′ cleavage product cleaved from the nucleic acid site in the cellular genome. In some cases, the 3′ arm comprises a sequence complementary to a region 5′ to the DNA site. The region configured to hybridize to the region 5′ to the DNA site or the region with complementarity to a region 5′ to the DNA site can comprise at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35 nucleotides, at least 50 nucleotides, at least 100 nucleotides, at least 150 nucleotides, at least 200 nucleotides, at least 250 nucleotides, at least 300 nucleotides, at least 350 nucleotides, or at least 400 nucleotides. In some embodiments, the region configured to hybridize to the region 5′ to the DNA site or the region with complementarity to a region 5′ to the DNA site comprises a mismatch of at least 1, at least 2, at least 3, at least 4, at least 5, or at least 6 nucleotides. In some cases when the insert DNA comprises a mismatch, the Cas protein can comprise an inactivating mutation in one or both endonuclease domains. The insert DNA molecule can comprise an insert DNA sequence contiguous with the 3′ arm. The insert DNA sequence can comprise at least 1 nucleotide, at least 50 bp, at least 100 bp, at least 150 bp, at least 200 bp, at least 250 bp, at least 300 bp, at least 350 bp, at least 400 bp, at least 450 bp, at least 500 bp, at least 600 bp, at least 700 bp, at least 800 bp, at least 900 bp, at least 1000 bp, at least 1200 bp, at least 1400 bp, at least 1600 bp, at least 1800 bp, at least 2000 bp, at least 2500 bp, at least 3000 bp, at least 3500 bp, at least 4000 bp, at least 4500 bp, at least 5000 bp, at least 5500 bp, at least 6000 bp, at least 6500 bp, at least 7000 bp, at least 7500 bp, at least 8000 bp, at least 8500 bp, at least 9000 bp, at least 9500 bp, or at least 10,000 bp. The insert DNA molecule can comprise at least 1 nucleotide, at least 50 bp, at least 100 bp, at least 150 bp, at least 200 bp, at least 250 bp, at least 300 bp, at least 350 bp, at least 400 bp, at least 450 bp, at least 500 bp, at least 600 bp, at least 700 bp, at least 800 bp, at least 900 bp, at least 1000 bp, at least 1200 bp, at least 1400 bp, at least 1600 bp, at least 1800 bp, at least 2000 bp, at least 2500 bp, at least 3000 bp, at least 3500 bp, at least 4000 bp, at least 4500 bp, at least 5000 bp, at least 5500 bp, at least 6000 bp, at least 6500 bp, at least 7000 bp, at least 7500 bp, at least 8000 bp, at least 8500 bp, at least 9000 bp, at least 9500 bp, or at least 10,000 bp aside from the region complementary or the region configured to hybridize. The insert DNA molecule can be single stranded deoxyribonucleic acid molecule, at least partially a single-stranded deoxyribonucleic acid molecule, or at least partially a double-stranded deoxyribonucleic acid molecule. The insert DNA molecule can be linked to the class 2, type V Cas endonuclease. The insert DNA molecule can comprise a hybridization domain configured to hybridize to a region of a guide polynucleotide. In some cases, the hybridization domain can comprise at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 nucleotides. The insert DNA molecule can be linked to a guide polynucleotide configured to interact with the class 2, type V Cas endonuclease. The insert DNA molecule can be hybridized to a guide polynucleotide configured to interact with the class 2, type V Cas endonuclease.

In some cases, the system enables an improved efficiency of insertion of the insert DNA molecule. In some cases, the composition allows for at least about a 1%, 2%, 5%, 10%, 15%, 20%, 25%, 30%, 35%, 40%, 45%, or 50% efficiency of insertion of said insert DNA into a cell.

The system can comprise a polypeptide with polymerase activity linked to the Cas endonuclease. Accordingly, the polypeptide with polymerase activity can comprise a DNA polymerase or a functional fragment thereof. DNA polymerases suitable for use with the methods and compositions described herein include, but are not limited to, T7 DNA polymerase, Bst polymerase or analogs thereof, a T4 DNA polymerase, Taq polymerase, Vent polymerase, Q5 polymerase, Klenow fragment, Phi29 polymerase, functional fragments thereof, or combinations thereof. The DNA polymerase can be an isothermal DNA polymerase, such as Bst polymerase or Bst2.0 polymerase. The polypeptide with polymerase activity can be linked N-terminal to the programmable nuclease. The polypeptide with polymerase activity can be linked C-terminal to the programmable nuclease. The polypeptide with polypeptide with polymerase activity can be linked to the programmable nuclease using a linker between the polypeptide with a second enzymatic activity and the programmable nuclease. The linker can comprise a peptide bond, an isopeptide bond, a small molecule linker, or a combination thereof. The linker can comprise LPXTG (SEQ ID NO: 59), (GGG)ₙ (SEQ ID NO: 60), (GGGGS)ₙ (SEQ ID NO: 61), (GGGS)ₙ (SEQ ID NO: 62), N₁₋₇, a biotin-streptavidin pair or an analog of a biotin-streptavidin pair, or SpyTag/SpyCatcher sequences linked by an isopeptide bond.

In some aspects, the present disclosure provides for a composition comprising: (a) a programmable nuclease configured to bind a double-stranded DNA site; and (b) a polypeptide having DNA topoisomerase activity linked to the programmable nuclease. In some cases, the polypeptide having DNA topoisomerase activity contains a catalytic hydroxyl group linked to an insert DNA template. In some cases, the programmable nuclease is configured to cleave at least one strand of DNA at the double-stranded DNA site. In some cases, the programmable nuclease is configured to cleave both strands of DNA at the double-stranded DNA site. The programmable nuclease can comprise a Cas protein or a Transcription activator-like effector nuclease (TALEN). Cas proteins suitable for the methods described herein include, but are not limited to, Class 2, Type II Cas proteins and a Class 2, Type V Cas proteins, including e.g., Cas9 proteins, Cas12a proteins, Cas12b proteins, Cas12c proteins, Cas12d proteins, Cas12e proteins, Cas 12f proteins, C2C10 proteins, Cas14ab proteins, Type V-U1 proteins, Type V-U2 proteins, Type V-U3 proteins, Type V-U4 proteins, Type V-U5 proteins, derivatives thereof, or hybrids thereof. The programmable nuclease can comprise a Transcription activator-like effector nuclease (TALEN). The Cas protein can comprise an inactivating mutation in one or both endonuclease domains. The Cas protein can comprise an inactivating mutation in a RuvC domain, an HNH domain, or both RuvC and HNH domains. The TALEN can comprise at least one, at least two, at least three, at least four, at least five, at least six, at least seven, at least 8, at least 9, or at least 10 Transcription activator-like effector (TAL) DNA-binding domains fused to an endonuclease domain. The endonuclease domain can comprise an endonuclease specific for four or fewer, three or fewer, two or fewer, one or fewer, or no nucleotide residues. The endonuclease domain can comprise a FokI endonuclease domain or a PvuII endonuclease domain. In some embodiments the fusion comprises a non-LTR retrotransposon polymerase-endonuclease.

The programmable nuclease can comprise a Cas protein or a Transcription activator-like effector nuclease (TALEN). Cas proteins suitable for the methods described herein include, but are not limited to, Class 2, Type II Cas proteins and a Class 2, Type V Cas proteins, including e.g., Cas9 proteins, Cas12a proteins, Cas12b proteins, Cas12c proteins, Cas12d proteins, Cas12e proteins, Cas 12f proteins, C2C10 proteins, Cas14ab proteins, Type V-U1 proteins, Type V-U2 proteins, Type V-U3 proteins, Type V-U4 proteins, Type V-U5 proteins, derivatives thereof, or hybrids thereof. The programmable nuclease can comprise a Transcription activator-like effector nuclease (TALEN). The Cas protein can comprise an inactivating mutation in one or both endonuclease domains. The Cas protein can comprise an inactivating mutation in a RuvC domain, an HNH domain, or both RuvC and HNH domains. The TALEN can comprise at least one, at least two, at least three, at least four, at least five, at least six, at least seven, at least 8, at least 9, or at least 10 Transcription activator-like effector (TAL) DNA-binding domains fused to an endonuclease domain. The endonuclease domain can comprise an endonuclease specific for four or fewer, three or fewer, two or fewer, one or fewer, or no nucleotide residues. The endonuclease domain can comprise a FokI endonuclease domain or a PvuII endonuclease domain.

When the programmable nuclease is a Cas enzyme, the composition comprising the programmable nuclease can further comprise a guide polynucleotide configured to interact with the Cas protein, wherein the guide polynucleotide is configured to hybridize to the double-stranded DNA site. The guide polynucleotide can comprise DNA, RNA, or a combination thereof. The guide nucleic acid can comprise a nucleic acid-targeting sequence and a Cas-binding sequence. The nucleic acid targeting sequence can comprise at least about 19-21 nucleotides in length that are configured to hybridize to the double-stranded DNA site. In some cases, the guide polynucleotide can comprise the nucleic acid-targeting sequence at a first end and a second end comprising a hybridization domain capable of hybridizing to at least one strand of an insert DNA molecule. In some cases, the hybridization domain is at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 nucleotides.

In some cases, the composition further comprises an insert DNA molecule. The insert DNA molecule can comprise a region homologous to a region 5′ to the double-stranded DNA site. The insert DNA molecule can comprise a region identical to a region 5′ to the double-stranded DNA site. The insert DNA molecule can comprise a region with at least 80%, 81%, 82%, 83%, 84%, 85%, 86%, 87%, 88%, 89%, 90%, 91%, 92%, 93%, 94%, 95%, 96%, 97%, 98%, or 99% identity to a region 5′ to the double-stranded DNA site. The region homologous to the region 5′ to the double-stranded DNA site or the region with complementarity to a region 5′ to the double-stranded DNA site can comprise at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35 nucleotides, at least 50 nucleotides, at least 100 nucleotides, at least 150 nucleotides, at least 200 nucleotides, at least 250 nucleotides, at least 300 nucleotides, at least 350 nucleotides, or at least 400 nucleotides. In some embodiments, the region homologous to the region 5′ to the double-stranded DNA site or the region with complementarity to a region 5′ to the double-stranded DNA site comprises a mismatch of at least 1, at least 2, at least 3, at least 4, at least 5, or at least 6 nucleotides. The insert DNA molecule can comprise at least 1 nucleotide, at least 50 bp, at least 100 bp, at least 150 bp, at least 200 bp, at least 250 bp, at least 300 bp, at least 350 bp, at least 400 bp, at least 450 bp, at least 500 bp, at least 600 bp, at least 700 bp, at least 800 bp, at least 900 bp, at least 1000 bp, at least 1200 bp, at least 1400 bp, at least 1600 bp, at least 1800 bp, at least 2000 bp, at least 2500 bp, at least 3000 bp, at least 3500 bp, at least 4000 bp, at least 4500 bp, at least 5000 bp, at least 5500 bp, at least 6000 bp, at least 6500 bp, at least 7000 bp, at least 7500 bp, at least 8000 bp, at least 8500 bp, at least 9000 bp, at least 9500 bp, or at least 10,000 bp. The insert DNA molecule can comprise at least 1 nucleotide, at least 50 bp, at least 100 bp, at least 150 bp, at least 200 bp, at least 250 bp, at least 300 bp, at least 350 bp, at least 400 bp, at least 450 bp, at least 500 bp, at least 600 bp, at least 700 bp, at least 800 bp, at least 900 bp, at least 1000 bp, at least 1200 bp, at least 1400 bp, at least 1600 bp, at least 1800 bp, at least 2000 bp, at least 2500 bp, at least 3000 bp, at least 3500 bp, at least 4000 bp, at least 4500 bp, at least 5000 bp, at least 5500 bp, at least 6000 bp, at least 6500 bp, at least 7000 bp, at least 7500 bp, at least 8000 bp, at least 8500 bp, at least 9000 bp, at least 9500 bp, or at least 10,000 bp aside from the region homologous. The insert DNA molecule can be single stranded deoxyribonucleic acid molecule, at least partially a single-stranded deoxyribonucleic acid molecule, or at least partially a double-stranded deoxyribonucleic acid molecule. The insert DNA molecule can be linked to the polypeptide with topoisomerase activity. The insert DNA molecule can be linked to the catalytic hydroxyl group of the polypeptide having DNA topoisomerase activity at a first end. The first end can be a 5′ end or a 3′ end. The insert DNA molecule can comprise the region homologous to a region 5′ to the nucleic acid site or the region homologous to a region 3′ to the nucleic acid site at a second end. The second end can be a 5′ end or a 3′ end.

In some cases, the composition enables an improved efficiency of insertion of the insert DNA molecule. In some cases, the composition allows for at least about a 1%, 2%, 5%, 10%, 15%, 20%, 25%, 30%, 35%, 40%, 45%, or 50% efficiency of insertion of said insert DNA into a cell.

The polypeptide having DNA topoisomerase activity linked to the programmable nuclease can be configured in a variety of ways. The polypeptide having DNA topoisomerase activity can be linked N-terminal to the programmable nuclease. The polypeptide having DNA topoisomerase activity can be linked C-terminal to the programmable nuclease. The polypeptide having DNA topoisomerase activity can be linked N-terminal or C-terminal to the programmable nuclease via a linker molecule. The linker molecule can comprise a peptide bond, an isopeptide bond, a small molecule linker, or a combination thereof. The linker can comprise LPXTG (SEQ ID NO: 59), (GGG)ₙ (SEQ ID NO: 60), (GGGGS)ₙ (SEQ ID NO: 61), (GGGS)ₙ (SEQ ID NO: 62), N₁₋₇, a biotin-streptavidin pair, or an analog of a biotin-streptavidin pair, or SpyTag/SpyCatcher sequences linked by an isopeptide bond. The polypeptide having DNA topoisomerase activity can comprise a topoisomerase enzyme or a functional fragment thereof. The topoisomerase can comprise a Type I topoisomerase or a Type II topoisomerase. Type I topoisomerases can include Type 1A topoisomerases, such as e.g., E. coli Eubacterial DNA topoisomerase I, E. coli Eubacterial DNA topoisomerase III, S. cerevisiae Yeast DNA topoisomerase IIII, H. sapiens DNA topoisomerase IIIα or IIIβ, S. acidocaldarius eubacterial and archaeal reverse DNA gyrase, or M. kandleri eubacterial reverse gyrase. Type I topoisomerases can include Type 1B topoisomerases, such as e.g., H. sapiens eukaryotic DNA topoisomerase I, Vaccinia poxvirus topoisomerase I, or M. kandleri hyperthermophilic eubacterial DNA topoisomerase V. Type II topoisomerases can comprise Type IIA topoisomerases such as e.g., E. coli eubacterial DNA gyrase, E. coli eubacterial DNA topoisomerase IV, S. cerevisiae yeast DNA topoisomerase II, or H. sapiens mammalian DNA topoisomerase IIαor IIβ. Type II topoisomerases can comprise Type IIB topoisomerases such as e.g., S. shibatae archaeal DNA topoisomerase VI.

### Displacement-Loop Forming Compositions

In some aspects, the present disclosure provides for a composition comprising a complex having the following linked components: (a) a polynucleotide comprising a region homologous to a nucleic acid site in a cellular genome; (b) a displacement annealing domain; and (c) a polypeptide with DNA polymerase activity. In some cases, the displacement annealing domain can comprise (i) a polypeptide having RecA-like activity; and (ii) at least one polypeptide having RecN-like activity. In some cases, the displacement annealing domain comprises from N- to C- terminus: at least one first polypeptide with RecA-like activity, an optional first linker, a polypeptide having RecN-like activity, an optional second linker, and at least one second polypeptide having RecA-like activity. In some cases, the at least one first polypeptide with RecA-like activity comprises at least two, at least three, at least four, or at least five polypeptides with Rec-A like activity. In some cases, the at least one second polypeptide with RecA-like activity comprises at least two, at least three, at least four, or at least five polypeptides with Rec-A like activity. In some cases, the complex having the linked components comprises the following polypeptides from N- to C-terminus: a polypeptide with Rec A-like activity, a polypeptide with RecN-like activity, a polypeptide with RecA-like activity, and a polypeptide with DNA polymerase activity. The polypeptide with RecA-like activity can be RecA from E. coli or Rad54 from H. sapiens. The polypeptide with RecN-like activity can be RecN from E. coli or Rad51 from H. sapiens.

The polypeptide with DNA polymerase activity can be configured in a variety of ways. The polypeptide with DNA polymerase activity can comprise a DNA polymerase or a functional fragment thereof. DNA polymerases suitable for use with compositions and methods herein include e.g., T7 DNA polymerase, Bst polymerase or an analog thereof, T4 DNA polymerase, Taq polymerase, Vent polymerase, Q5 polymerase, Klenow fragment, Phi29 polymerase, a functional fragment thereof, or any combination thereof. The polypeptide with DNA polymerase activity can be linked N-terminal to the programmable nuclease. The polypeptide with DNA polymerase activity can be linked C-terminal to the programmable nuclease. The polypeptide with DNA polymerase activity can be linked N-terminal or C-terminal to the programmable nuclease via a linker molecule. The linker molecule can comprise a peptide bond, an isopeptide bond, a small molecule linker, or a combination thereof. The linker can comprise LPXTG (SEQ ID NO: 59), (GGG)ₙ (SEQ ID NO: 60), (GGGGS)ₙ (SEQ ID NO: 61), (GGGS)ₙ (SEQ ID NO: 62), N₁₋₇, a biotin-streptavidin pair, or an analog of a biotin-streptavidin pair, or SpyTag/SpyCatcher sequences linked by an isopeptide bond.

The polynucleotide comprising the region homologous to the nucleic acid site in the cellular genome can be configured in a variety of ways. The region homologous to the nucleic acid site in a cellular genome comprises at least 10, at least 20, at least 30, at least 40, or at least 50 base pairs. The region homologous to the nucleic acid site in the cellular genome can comprise a region identical to the nucleic acid site. The region homologous to the nucleic acid site in the cellular genome can comprise a region homologous to the nucleic acid site. The region homologous to the nucleic acid site in the cellular genome can comprise a region with at least 80%, 81%, 82%, 83%, 84%, 85%, 86%, 87%, 88%, 89%, 90%, 91%, 92%, 93%, 94%, 95%, 96%, 97%, 98%, or 99% identity to the nucleic acid site. The polynucleotide comprising the region homologous to the nucleic acid site in the cellular genome can be linked to an N-terminus or C-terminus of the displacement annealing domain. The polynucleotide comprising the region homologous to the nucleic acid site in the cellular genome can be linked to an N-terminus or a C-terminus of the polypeptide with DNA polymerase activity. In some cases, the polynucleotide comprising a region homologous to a nucleic acid site in a cellular genome further comprises an insert nucleic acid sequence comprising at least about 1 bp to at least about 20 kb. The insert DNA molecule can comprise at least 1 nucleotide, at least 50 bp, at least 100 bp, at least 150 bp, at least 200 bp, at least 250 bp, at least 300 bp, at least 350 bp, at least 400 bp, at least 450 bp, at least 500 bp, at least 600 bp, at least 700 bp, at least 800 bp, at least 900 bp, at least 1000 bp, at least 1200 bp, at least 1400 bp, at least 1600 bp, at least 1800 bp, at least 2000 bp, at least 2500 bp, at least 3000 bp, at least 3500 bp, at least 4000 bp, at least 4500 bp, at least 5000 bp, at least 5500 bp, at least 6000 bp, at least 6500 bp, at least 7000 bp, at least 7500 bp, at least 8000 bp, at least 8500 bp, at least 9000 bp, at least 9500 bp, or at least 10,000 bp. The insert DNA molecule can comprise at least 1 nucleotide, at least 50 bp, at least 100 bp, at least 150 bp, at least 200 bp, at least 250 bp, at least 300 bp, at least 350 bp, at least 400 bp, at least 450 bp, at least 500 bp, at least 600 bp, at least 700 bp, at least 800 bp, at least 900 bp, at least 1000 bp, at least 1200 bp, at least 1400 bp, at least 1600 bp, at least 1800 bp, at least 2000 bp, at least 2500 bp, at least 3000 bp, at least 3500 bp, at least 4000 bp, at least 4500 bp, at least 5000 bp, at least 5500 bp, at least 6000 bp, at least 6500 bp, at least 7000 bp, at least 7500 bp, at least 8000 bp, at least 8500 bp, at least 9000 bp, at least 9500 bp, or at least 10,000 bp.

TABLE 1
Sequences of Genes and Components Described Herein

| SEQ | | |
|---|---|---|
| ID | | |
| NO: | TDESCRIPION | SEQUENCE |
| 1 | GE-1 | CGAACUGAAAAGCCCACUGGACA UAU |
| | primer | AACTGTTGGGAAGGGCGA |
| 2 | GE-2 | CGTATTACCGCCTTTGAGTGAG |
| | primer | |
| 3 | GE-3 | GCGCGGTATTATCCCGTATTGA |
| | primer | |
| 4 | GE-4 | TGAAACGCTTGCTGCAACG |
| | primer | |
| 5 | GE-5 | GTACAAAAGCGGTGTTCGCAATC |
| | primer | |
| 6 | GE-6 | GTTACCCAACTTAATCGCCTTGCAG |
| | primer | |
| 7 | GE-7 | TGCGTATTGGGCGCTCTTC |
| | primer | |
| 8 | sgRNA | mG*mA*mA* rArArG rCrCrC rArCrU rGrGrA rCrArG |
| | GE-8 | rUrCrG rUrUrU rUrArG rArGrC rUrArG rArArA |
| | | rUrArG rCrArA rGrUrU rArArA rArUrA rArGrG |
| | | rCrUrA rGrUrC rCrGrU rUrArU rCrArA rCrUrU |
| | | rGrArA rArArA rGrUrG rGrCrA rCrCrG rArGrU |
| | | rCrGrG rUrGrC mU*mU*mU* rU |
| 9 | sgRNA | mG*mA*mA* rArArG rCrCrC rArCrU rGrGrA rCrArG |
| | GE-9 | rUrC rG rUrUrU rUrArG rArGrC rUrArG rArArA |
| | | rUrArG rCrArA rGrUrU rArArA rArUrA rArGrG |
| | | rCrUrA rGrUrC rCrGrU rUrArU rCrArA rCrUrU |
| | | rGrArA rArArA rGrUrG rGrCrA rCrCrG rArGrU |
| | | rCrGrG rUrGrC rGrCrU rArGrU rUrArU rUrGrC |
| | | rUrCrA rGrGrC rCrCrG mU*mU* rU |
| 10 | G-Block | aagcTAATACGACTCACTATAGGAAAAGCCCACTGGACAGTCGT |
| | primer | TTTAGAGCTAGAAATAGCAAGTTAAAATAA |
| | | GGCTAGTCCGTTATCAACTTGAAAAAGTGGCACCGAGTCGGTGC |
| | | GCT AGT TAT TGC TCA GGC CCG TTTT |
| | | rG rA rArArG rCrCrC rArCrU rGrGrA rCrArG |
| | | rUrCrG rUrUrU rUrArG rArGrC rUrArG rArArA |
| | | rUrArG rCrArA rGrUrU rArArA rArUrA rArGrG |
| | | rCrUrA rGrUrC rCrGrU rUrArU rCrArA rCrUrU |
| | | rGrArA rArArA rGrUrG rGrCrA rCrCrG rArGrU |
| | | rCrGrG rUrGrC rGrCrU rArGrU rUrArU rUrGrC |
| | | rUrCrA rGrGrC rCrCrG rU |
| 11 | GE-10 | AACTGTTGGGAAGGGCGATCGGTGCGGGCCTCTTCGCTATTACG |
| | | CCAGCTGGCGAAAGGGGGATGTGCTGCAAGGCGATTAAGTTGGG |
| | | TAACGCCAGGGT CGG GCC TGA GCA ATA ACT AGC |
| 12 | GE-11 | ACCCTGGCGTTACCCAACTTAATCGCCTTGCAGCACATCCCCCT |
| | | TTCGCCAGCTGGCGTAATAGCGAAGAGGCCCGCACCGATCGCCC |
| | | TTCCCAACAGTT TTTTTT TGTCCAGTGGGCTTTTCAGTTCG |
| 13 | GE-20 | GAAGCGGCGGCAATACGT |
| 14 | SpCas9 | MDKKYSIGLDIGTNSVGWAVITDEYKVPSKKFKVLGNTDRHSIK |
| | sequence | KNLIGALLFDSGETAEATRLKRTARRRYTRRKNRICYLQEIFSN |
| | | EMAKVDDSFFHRLEESFLVEEDKKHERHPIFGNIVDEVAYHEKY |
| | | PTIYHLRKKLVDSTDKADLRLIYLALAHMIKFRGHFLIEGDLNP |
| | | DNSDVDKLFIQLVQTYNQLFEENPINASGVDAKAILSARLSKSR |
| | | RLENLIAQLPGEKKNGLFGNLIALSLGLTPNFKSNFDLAEDAKL |
| | | QLSKDTYDDDLDNLLAQIGDQYADLFLAAKNLSDAILLSDILRV |
| | | NTEITKAPLSASMIKRYDEHHQDLTLLKALVRQQLPEKYKEIFF |
| | | DQSKNGYAGYIDGGASQEEFYKFIKPILEKMDGTEELLVKLNRE |
| | | DLLRKQRTFDNGSIPHQIHLGELHAILRRQEDFYPFLKDNREKI |
| | | EKILTFRIPYYVGPLARGNSRFAWMTRKSEETITPWNFEEVVDK |
| | | GASAQSFIERMTNFDKNLPNEKVLPKHSLLYEYFTVYNELTKVK |
| | | YVTEGMRKPAFLSGEQKKAIVDLLFKTNRKVTVKQLKEDYFKKI |
| | | ECFDSVEISGVEDRFNASLGTYHDLLKIIKDKDFLDNEENEDIL |
| | | EDIVLTLTLFEDREMIEERLKTYAHLFDDKVMKQLKRRRYTGWG |
| | | RLSRKLINGIRDKQSGKTILDFLKSDGFANRNFMQLIHDDSLTF |
| | | KEDIQKAQVSGQGDSLHEHIANLAGSPAIKKGILQTVKVVDELV |
| | | KVMGRHKPENIVIEMARENQTTQKGQKNSRERMKRIEEGIKELG |
| | | SQILKEHPVENTQLQNEKLYLYYLQNGRDMYVDQELDINRLSDY |
| | | DVDHIVPQSFLKDDSIDNKVLTRSDKNRGKSDNVPSEEVVKKMK |
| | | NYWRQLLNAKLITQRKFDNLTKAERGGLSELDKAGFIKRQLVET |
| | | RQITKHVAQILDSRMNTKYDENDKLIREVKVITLKSKLVSDFRK |
| | | DFQFYKVREINNYHHAHDAYLNAVVGTALIKKYPKLESEFVYGD |
| | | YKVYDVRKMIAKSEQEIGKATAKYFFYSNIMNFFKTEITLANGE |
| | | IRKRPLIETNGETGEIVWDKGRDFATVRKVLSMPQVNIVKKTEV |
| | | QTGGFSKESILPKRNSDKLIARKKDWDPKKYGGFDSPTVAYSVL |
| | | VVAKVEKGKSKKLKSVKELLGITIMERSSFEKNPIDFLEAKGYK |
| | | EVKKDLIIKLPKYSLFELENGRKRMLASAGELQKGNELALPSKY |
| | | VNFLYLASHYEKLKGSPEDNEQKQLFVEQHKHYLDEIIEQISEF |
| | | SKRVILADANLDKVLSAYNKHRDKPIREQAENIIHLFTLTNLGA |
| | | PAAFKYFDTTIDRKRYTSTKEVLDATLIHQSITGLYETRIDLSQ |
| | | LGGD |
| 15 | SauCas9 | MKRNYILGLDIGITSVGYGIIDYETRDVIDAGVRLFKEANVENN |
| | | EGRRSKRGARRLKRRRRHRIQRVKKLLFDYNLLTDHSELSGINP |
| | | YEARVKGLSQKLSEEEFSAALLHLAKRRGVHNVNEVEEDTGNEL |
| | | STKEQISRNSKALEEKYVAELQLERLKKDGEVRGSINRFKTSDY |
| | | VKEAKQLLKVQKAYHQLDQSFIDTYIDLLETRRTYYEGPGEGSP |
| | | FGWKDIKEWYEMLMGHCTYFPEELRSVKYAYNADLYNALNDLNN |
| | | LVITRDENEKLEYYEKFQIIENVFKQKKKPTLKQIAKEILVNEE |
| | | DIKGYRVTSTGKPEFTNLKVYHDIKDITARKEIIENAELLDQIA |
| | | KILTIYQSSEDIQEELTNLNSELTQEEIEQISNLKGYTGTHNLS |
| | | LKAINLILDELWHTNDNQIAIFNRLKLVPKKVDLSQQKEIPTTL |
| | | VDDFILSPVVKRSFIQSIKVINAIIKKYGLPNDIIIELAREKNS |
| | | KDAQKMINEMQKRNRQTNERIEEIIRTTGKENAKYLIEKIKLHD |
| | | MQEGKCLYSLEAIPLEDLLNNPFNYEVDHIIPRSVSFDNSFNNK |
| | | VLVKQEENSKKGNRTPFQYLSSSDSKISYETFKKHILNLAKGKG |
| | | RISKTKKEYLLEERDINRFSVQKDFINRNLVDTRYATRGLMNLL |
| | | RSYFRVNNLDVKVKSINGGFTSFLRRKWKFKKERNKGYKHHAED |
| | | ALIIANADFIFKEWKKLDKAKKVMENQMFEEKQAESMPEIETEQ |
| | | EYKEIFITPHQIKHIKDFKDYKYSHRVDKKPNRELINDTLYSTR |
| | | KDDKGNTLIVNNLNGLYDKDNDKLKKLINKSPEKLLMYHHDPQT |
| | | YQKLKLIMEQYGDEKNPLYKYYEETGNYLTKYSKKDNGPVIKKI |
| | | KYYGNKLNAHLDITDDYPNSRNKVVKLSLKPYRFDVYLDNGVYK |
| | | FVTVKNLDVIKKENYYEVNSKCYEEAKKLKKISNQAEFIASFYN |
| | | NDLIKINGELYRVIGVNNDLLNRIEVNMIDITYREYLENMNDKR |
| | | PPRIIKTIASKTQSIKKYSTDILGNLYEVKSKKHPQIIKKG |
| 16 | Bst2.0 | EGEKPLAGMDFAIADSVTDEMLADKAALVVEVVGDNYHHAPIVG |
| | polymerase | IALANERGRFFLRPETALADPKFLAWLGDETKKKTMFDSKRAAV |
| | sequence | ALKWKGIELRGVVFDLLLAAYLLDPAQAAGDVAAVAKMHQYEAV |
| | | RSDEAVYGKGAKRTVPDEPTLAEHLVRKAAAIWALEEPLMDELR |
| | | RNEQDRLLTELEQPLAGILANMEFTGVKVDTKRLEQMGAELTEQ |
| | | LQAVERRIYELAGQEFNINSPKQLGTVLFDKLQLPVLKKTKTGY |
| | | STSADVLEKLAPHHEIVEHILHYRQLGKLQSTYIEGLLKVVHPV |
| | | TGKVHTMFNQALTQTGRLSSVEPNLQNIPIRLEEGRKIRQAFVP |
| | | SEPDWLIFAADYSQIELRVLAHIAEDDNLIEAFRRGLDIHTKTA |
| | | MDIFHVSEEDVTANMRRQAKAVNFGIVYGISDYGLAQNLNITRK |
| | | EAAEFIERYFASFPGVKQYMDNIVQEAKQKGYVTTLLHRRRYLP |
| | | DITSRNFNVRSFAERTAMNTPIQGSAADIIKKAMIDLSVRLREE |
| | | RLQARLLLQVHDELILEAPKEEIERLCRLVPEVMEQAVTLRVPL |
| | | KVDYHYGPTWYDAK |
| 17 | Spy Tag | AHIVMVDAYKPTKKG |
| | sequence | |
| 18 | Spycatcher | DYDIPTTENLYFQGAMVDTLSGLSSEQGQSGDMTIEEDSATHIK |
| | sequence | FSKRDEDGKELAGATMELRDSSGKTISTWISDGQVKDFYLYPGK |
| | | YTFVETAAPDGYEVATAITFTVNEQGQVTVNGKATKGDAHI |
| 19 | Vaccinia | RALFYKDGKLFTDNNFLNPVSDDNPAYEVLQHVKIPTHLTDVVV |
| | Topoisomerase | YEQTWEEALTRLIFVGSDSKGRRQYFYGKMHVQNRNAKRDRIFV |
| | sequence | RVYNVMKRINCFINKNIKKSSTDSNYQLAVFMLMETMFFIRFGK |
| | | MKYLKENETVGLLTLKNKHIEISPDEIVIKFVGKDKVSHEFVVH |
| | | KSNRLYKPLLKLTDDSSPEEFLFNKLSERKVYECIKQFGIRIKD |
| | | LRTYGVNYTFLYNFWTNVKSISPLPSPKKLIALTIKQTAEVVGH |
| | | TPSISKRAYMATTILEMVKDKNFLDVVSKTTFDEF |
| | | LSIVVDHVKSSTDG |
| 20 | DNA | MRLFIAEKPSLARAIADVLPKPHRKGDGFIECGNGQVVTWCIGH |
| | topoisomerase | LLEQAQPDAYDSRYARWNLADLPIVPEKWQLQPRPSVTKQLNVI |
| | III (E. coli) | KRFLHEASEIVHAGDPDREGQLLVDEVLDYLQLAPEKRQQVQRC |
| | | LINDLNPQAVERAIDRLRSNSEFVPLCVSALARARADWLYGINM |
| | | TRAYTILGRNAGYQGVLSVGRVQTPVLGLVVRRDEEIENFVAKD |
| | | FFEVKAHIVTPADERFTAIWQPSEACEPYQDEEGRLLHRPLAEH |
| | | VVNRISGQPAIVTSYNDKRESESAPLPFSLSALQIEAAKRFGLS |
| | | AQNVLDICQKLYETHKLITYPRSDCRYLPEEHFAGRHAVMNAIS |
| | | VHAPDLLPQPVVDPDIRNRCWDDKKVDAHHAIIPTARSSAINLT |
| | | ENEAKVYNLIARQYLMQFCPDAVFRKCVIELDIAKGKFVAKARF |
| | | LAEAGWRTLLGSKERDEENDGTPLPVVAKGDELLCEKGEVVERQ |
| | | TQPPRHFTDATLLSAMTGIARFVQDKDLKKILRATDGLGTEATR |
| | | AGIIELLFKRGFLTKKGRYIHSTDAGKALFHSLPEMATRPDMTA |
| | | HWESVLTQISEKQCRYQDFMQPLVGTLYQLIDQAKRTPVRQFRG |
| | | IVAPGSGGSADKKKAAPRKRSAKKSPPADEVGSGAIA |
| 21 | DNA | MGKALVIVESPAKAKTINKYLGSDYVVKSSVGHIRDLPTSGSAA |
| | topoisomerase | KKSADSTSTKTAKKPKKDERGALVNRMGVDPWHNWEAHYEVLPG |
| | type | KEKVVSELKQLAEKADHIYLATDLDREGEAIAWHLREVIGGDDA |
| | I(E. coli) | RYSRVVFNEITKNAIRQAFNKPGELNIDRVNAQQARRFMDRVVG |
| | | YMVSPLLWKKIARGLSAGRVQSVAVRLVVEREREIKAFVPEEFW |
| | | EVDASTTTPSGEALALQVTHQNDKPFRPVNKEQTQAAVSLLEKA |
| | | RYSVLEREDKPTTSKPGAPFITSTLQQAASTRLGFGVKKTMMMA |
| | | QRLYEAGYITYMRTDSTNLSQDAVNMVRGYISDNFGKKYLPESP |
| | | NQYASKENSQEAHEAIRPSDVNVMAESLKDMEADAQKLYQLIWR |
| | | QFVACQMTPAKYDSTTLTVGAGDFRLKARGRILRFDGWTKVMPA |
| | | LRKGDEDRILPAVNKGDALTLVELTPAQHFTKPPARFSEASLVK |
| | | ELEKRGIGRPSTYASIISTIQDRGYVRVENRRFYAEKMGEIVTD |
| | | RLEENFRELMNYDFTAQMENSLDQVANHEAEWKAVLDHFFSDFT |
| | | QQLDKAEKDPEEGGMRPNQMVLTSIDCPTCGRKMGIRTASTGVF |
| | | LGCSGYALPPKERCKTTINLVPENEVLNVLEGEDAETNALRAKR |
| | | RCPKCGTAMDSYLIDPKRKLHVCGNNPTCDGYEIEEGEFRIKGY |
| | | DGPIVECEKCGSEMHLKMGRFGKYMACTNEECKNTRKILRNGEV |
| | | APPKEDPVPLPELPCEKSDAYFVLRDGAAGVFLAANTFPKSRET |
| | | RAPLVEELYRFRDRLPEKLRYLADAPQQDPEGNKTMVRFSRKTK |
| | | QQYVSSEKDGKATGWSAFYVDGKWVEGKK |
| 22 | RecN | MLAQLTISNFAIVRELEIDFHSGMTVITGETGAGKSIAIDALGL |
| | sequence | CLGGRAEADMVRTGAARADLCARFSLKDTPAALRWLEENQLEDG |
| | | HECLLRRVISSDGRSRGFINGTAVPLSQLRELGQLLIQIHGQHA |
| | | HQLLTKPEHQKFLLDGYANETSLLQEMTARYQLWHQSCRDLAHH |
| | | QQLSQERAARAELLQYQLKELNEFNPQPGEFEQIDEEYKRLANS |
| | | GQLLTTSQNALALMADGEDANLQSQLYTAKQLVSELIGMDSKLS |
| | | GVLDMLEEATIQIAEASDELRHYCDRLDLDPNRLFELEQRISKQ |
| | | ISLARKHHVSPEALPQYYQSLLEEQQQLDDQADSQETLALAVTK |
| | | HHQQALEIARALHQQRQQYAEELAQLITDSMHALSMPHGQFTID |
| | | VKFDEHHLGADGADRIEFRVTTNPGQPMQPIAKVASGGELSRIA |
| | | LAIQVITARKMETPALIFDEVDVGISGPTAAVVGKLLRQLGEST |
| | | QVMCVTHLPQVAGCGHQHYFVSKETDGAMTETHMQSLNKKARLQ |
| | | ELARLLGGSEVTRNTLANAKELLAA |
| 23 | RecA | MAIDENKQKALAAALGQIEKQFGKGSIMRLGEDRSMDVETISTG |
| | sequence | SLSLDIALGAGGLPMGRIVEIYGPESSGKTTLTLQVIAAAQREG |
| | | KTCAFIDAEHALDPIYARKLGVDIDNLLCSQPDTGEQALEICDA |
| | | LARSGAVDVIVVDSVAALTPKAEIEGEIGDSHMGLAARMMSQAM |
| | | RKLAGNLKQSNTLLIFINQIRMKIGVMFGNPETTTGGNALKFYA |
| | | SVRLDIRRIGAVKEGENVVGSETRVKVVKNKIAAPFKQAEFQIL |
| | | YGEGINFYGELVDLGVKEKLIEKAGAWYSYKGEKIGQGKANATA |
| | | WLKDNPETAKEIEKKVRELLLSNPNSTPDFSVDDSEGVAETNED |
| | | F |
| 24 | RAD51 | MAMQMQLEANADTSVEEESFGPQPISRLEQCGINANDVKKLEEA |
| | sequence | GFHTVEAVAYAPKKELINIKGISEAKADKILAEAAKLVPMGFTT |
| | | ATEFHQRRSEIIQITTGSKELDKLLQGGIETGSITEMFGEFRTG |
| | | KTQICHTLAVTCQLPIDRGGGEGKAMYIDTEGTFRPERLLAVAE |
| | | RYGLSGSDVLDNVAYARAFNTDHQTQLLYQASAMMVESRYALLI |
| | | VDSATALYRTDYSGRGELSARQMHLARFLRMLLRLADEFGVAVV |
| | | ITNQVVAQVDGAAMFAADPKKPIGGNIIAHASTTRLYLRKGRGE |
| | | TRICKIYDSPCLPEAEAMFAINADGVGDAKD |
| 25 | RAD54 | MARRRLPDRPPNGIGAGERPRLVPRPINVQDSVNRLTKPFRVPY |
| | sequence | KNTHIPPAAGRIATGSDNIVGGRSLRKRSATVCYSGLDINADEA |
| | | EYNSQDISFSQLTKRRKDALSAQRLAKDPTRLSHIQYTLRRSFT |
| | | VPIKGYVQRHSLPLTLGMKKKITPEPRPLHDPTDEFAIVLYDPS |
| | | VDGEMIVHDTSMDNKEEESKKMIKSTQEKDNINKEKNSQEERPT |
| | | QRIGRHPALMTNGVRNKPLRELLGDSENSAENKKKFASVPVVID |
| | | PKLAKILRPHQVEGVRFLYRCVTGLVMKDYLEAEAFNTSSEDPL |
| | | KSDEKALTESQKTEQNNRGAYGCIMADEMGLGKTLQCIALMWTL |
| | | LRQGPQGKRLIDKCIIVCPSSLVNNWANELIKWLGPNTLTPLAV |
| | | DGKKSSMGGGNTTVSQAIHAWAQAQGRNIVKPVLIISYETLRRN |
| | | VDQLKNCNVGLMLADEGHRLKNGDSLTFTALDSISCPRRVILSG |
| | | TPIQNDLSEYFALLSFSNPGLLGSRAEFRKNFENPILRGRDADA |
| | | TDKEITKGEAQLQKLSTIVSKFIIRRTNDILAKYLPCKYEHVIF |
| | | VNLKPLQNELYNKLIKSREVKKVVKGVGGSQPLRAIGILKKLCN |
| | | HPNLLNFEDEFDDEDDLELPDDYNMPGSKARDVQTKYSAKFSIL |
| | | ERFLHKIKTESDDKIVLISNYTQTLDLIEKMCRYKHYSAVRLDG |
| | | TMSINKRQKLVDRFNDPEGQEFIFLLSSKAGGCGINLIGANRLI |
| | | LMDPDWNPAADQQALARVWRDGQKKDCFIYRFISTGTIEEKIFQ |
| | | RQSMKMSLSSCVVDAKEDVERLFSSDNLRQLFQKNENTICETHE |
| | | TYHCKRCNAQGKQLKRAPAMLYGDATTWNHLNHDALEKTNDHLL |
| | | KNEHHYNDISFAFQYISH |
| 26 | SpCas9- | MDKKYSIGLDIGTNSVGWAVITDEYKVPSKKFKVLGNTDRHSIK |
| | Bstpol | KNLIGALLFDSGETAEATRLKRTARRRYTRRKNRICYLQEIFSN |
| | fusion | EMAKVDDSFFHRLEESFLVEEDKKHERHPIFGNIVDEVAYHEKY |
| | | PTIYHLRKKLVDSTDKADLRLIYLALAHMIKFRGHFLIEGDLNP |
| | | DNSDVDKLFIQLVQTYNQLFEENPINASGVDAKAILSARLSKSR |
| | | RLENLIAQLPGEKKNGLFGNLIALSLGLTPNFKSNFDLAEDAKL |
| | | QLSKDTYDDDLDNLLAQIGDQYADLFLAAKNLSDAILLSDILRV |
| | | NTEITKAPLSASMIKRYDEHHQDLTLLKALVRQQLPEKYKEIFF |
| | | DQSKNGYAGYIDGGASQEEFYKFIKPILEKMDGTEELLVKLNRE |
| | | DLLRKQRTFDNGSIPHQIHLGELHAILRRQEDFYPFLKDNREKI |
| | | EKILTFRIPYYVGPLARGNSRFAWMTRKSEETITPWNFEEVVDK |
| | | GASAQSFIERMTNFDKNLPNEKVLPKHSLLYEYFTVYNELTKVK |
| | | YVTEGMRKPAFLSGEQKKAIVDLLFKTNRKVTVKQLKEDYFKKI |
| | | ECFDSVEISGVEDRFNASLGTYHDLLKIIKDKDFLDNEENEDIL |
| | | EDIVLTLTLFEDREMIEERLKTYAHLFDDKVMKQLKRRRYTGWG |
| | | RLSRKLINGIRDKQSGKTILDFLKSDGFANRNFMQLIHDDSLTF |
| | | KEDIQKAQVSGQGDSLHEHIANLAGSPAIKKGILQTVKVVDELV |
| | | KVMGRHKPENIVIEMARENQTTQKGQKNSRERMKRIEEGIKELG |
| | | SQILKEHPVENTQLQNEKLYLYYLQNGRDMYVDQELDINRLSDY |
| | | DVDHIVPQSFLKDDSIDNKVLTRSDKNRGKSDNVPSEEVVKKMK |
| | | NYWRQLLNAKLITQRKFDNLTKAERGGLSELDKAGFIKRQLVET |
| | | RQITKHVAQILDSRMNTKYDENDKLIREVKVITLKSKLVSDFRK |
| | | DFQFYKVREINNYHHAHDAYLNAVVGTALIKKYPKLESEFVYGD |
| | | YKVYDVRKMIAKSEQEIGKATAKYFFYSNIMNFFKTEITLANGE |
| | | IRKRPLIETNGETGEIVWDKGRDFATVRKVLSMPQVNIVKKTEV |
| | | QTGGFSKESILPKRNSDKLIARKKDWDPKKYGGFDSPTVAYSVL |
| | | VVAKVEKGKSKKLKSVKELLGITIMERSSFEKNPIDFLEAKGYK |
| | | EVKKDLIIKLPKYSLFELENGRKRMLASAGELQKGNELALPSKY |
| | | VNFLYLASHYEKLKGSPEDNEQKQLFVEQHKHYLDEIIEQISEF |
| | | SKRVILADANLDKVLSAYNKHRDKPIREQAENIIHLFTLTNLGA |
| | | PAAFKYFDTTIDRKRYTSTKEVLDATLIHQSITGLYETRIDLSQ |
| | | LGGDGGGGSGGGGSGGGGSEGEKPLAGMDFAIADSVTDEMLADK |
| | | AALVVEVVGDNYHHAPIVGIALANERGRFFLRPETALADPKFLA |
| | | WLGDETKKKTMFDSKRAAVALKWKGIELRGVVFDLLLAAYLLDP |
| | | AQAAGDVAAVAKMHQYEAVRSDEAVYGKGAKRTVPDEPTLAEHL |
| | | VRKAAAIWALEEPLMDELRRNEQDRLLTELEQPLAGILANMEFT |
| | | GVKVDTKRLEQMGAELTEQLQAVERRIYELAGQEFNINSPKQLG |
| | | TVLFDKLQLPVLKKTKTGYSTSADVLEKLAPHHEIVEHILHYRQ |
| | | LGKLQSTYIEGLLKVVHPVTGKVHTMFNQALTQTGRLSSVEPNL |
| | | QNIPIRLEEGRKIRQAFVPSEPDWLIFAADYSQIELRVLAHIAE |
| | | DDNLIEAFRRGLDIHTKTAMDIFHVSEEDVTANMRRQAKAVNFG |
| | | IVYGISDYGLAQNLNITRKEAAEFIERYFASFPGVKQYMDNIVQ |
| | | EAKQKGYVTTLLHRRRYLPDITSRNFNVRSFAERTAMNTPIQGS |
| | | AADIIKKAMIDLSVRLREERLQARLLLQVHDELILEAPKEEIER |
| | | LCRLVPEVMEQAVTLRVPLKVDYHYGPTWYDAKEGADPKKKRKV |
| | | DPKKKRKV] |
| 66 | GE-390 | AGAGTTGCTAAAATTGGGCAAAAGATCATCATTAGAAGAGTTAC |
| | | AAAGTGGTGGAGGGAGTATGGTCAGCAAGGGAGCAGAGTTGTTT |
| | | ACAGGAATTGTGCCTATTTTGATCGAGCTTAATGGTGATGTCAA |
| | | TGGTCACAAGTTCAGCGTCTCTGGAGAGGGCGAAGGGGATGCCA |
| | | CATACGGTAAGCTTACGTTTGTTACCGGTAAAAGAAGCTGAGGA |
| | | TAAACTCAGCATAAATGATCCGCTTTTTGAGAGGCAGTGGCACT |
| | | TGGTCAATCCAAGTTTTCCTGGCAGTGATATAAATGTTCTTGAT |
| | | CTGTGGTACAATAATATTACAGGCGCAGGGGTCGTGGCTGCCAT |
| | | TGTTGATGATGGCCTTGACTACGAAAATGAAGACTTGAAGGATA |
| | | ATTTTTGCGCTGAAGGTTCTTGGGATTTCAACGACAATCGGGCC |
| | | TGAGCAATAACTAGCAAAAAA |
| 27 | GE-347 | AGAGTTGCTAAAATTGGGCAAAAGATCATCATTAGAAGAGTTAC |
| | | AAAGTGGTGGAGGGAGTATGGTCAGCAAGGGAGCAGAGTTGTTT |
| | | ACAGGAATTGTGCCTATTTTGATCGAGCTTAATGGTGATGTCAA |
| | | TGGTCACAAGTTCAGCGTCTCTGGAGAGGGCGAAGGGGATGCCA |
| | | CATACGGTAAGCTTACGTTAAAATTCATTTGCACAACAGGCAAG |
| | | TTACCTGTACCATGGCCCACACTTGTGACCACCTTAAGTTACGG |
| | | GGTTCAGTGCTTCTCTAGGTACCCTGATCACATGAAGCAGCACG |
| | | ATTTCTTCAAGTCTGCCATGCCTGAGGGATACATTCAGGAGAGA |
| | | ACCATATTCTTCGAGGATGACGGGAACTACAAGAGCAGGGCTGA |
| | | GGTCAAGTTCGAAGGGGATACACTGGTAAATAGGATCGAGTTGA |
| | | CCGGCACAGATTTCAAGGAAGATGGAAACATCCTGGGCAATAAG |
| | | ATGGAGTACAACTATAACGCCCACAATGTCTACATAATGACCGA |
| | | TAAAGCTAAGAATGGCATAAAGGTTAATTTCAAGATTAGGCACA |
| | | ACATAGAGGATGGGTCCGTGCAGCTTGCCGATCACTACCAGCAG |
| | | AATACGCCCATAGGAGATGGACCTGTGTTGTTACCTGATAACCA |
| | | CTATTTAAGTACTCAGAGTGCTTTATCAAAGGACCCTAACGAAA |
| | | AGAGGGATCACATGATCTACTTCGGGTTCGTCACAGCGGCAGCA |
| | | ATAACCCACGGCATGGATGAGCTTTACAAGTAAGGAGGGGGATT |
| | | GTTACCGGTAAAAGAAGCTGAGGATAAACTCAGCATAAATGATC |
| | | CGCTTTTTGAGAGGCAGTGGCACTTGGTCAATCCAAGTTTTCCT |
| | | GGCAGTGATATAAATGTTCTTGATCTGTGGTACAATAATATTAC |
| | | AGGCGCAGGGGTCGTGGCTGCCATTGTTGATGATGGCCTTGACT |
| | | ACGAAAATGAAGACTTGAAGGATAATTTTTGCGCTGAAGGTTCT |
| | | TGGGATTTCAACGACAATCGGGCCTGAGCAATAACTAGCAAAAA |
| | | A |
| 28 | GE-335 | AGAGTTGCTAAAATTGGGCAAAAGATCATCATTAGAAGAGTTAC |
| | | AGGGGGGGAGGTGGAAGTGGTGGAGGGAGTATGGTCAGCAAGGG |
| | | AGCAGAGTTGTTTACAGGAATTGTGCCTATTTTGATCGAGCTTA |
| | | ATGGTGATGTCAATGGTCACAAGTTCAGCGTCTCTGGAGAGGGC |
| | | GAAGGGGATGCCACATACGGTAAGCTTACGTTAAAATTCATTTG |
| | | CACAACAGGCAAGTTACCTGTACCATGGCCCACACTTGTGACCA |
| | | CCTTAAGTTACGGGGTTCAGTGCTTCTCTAGGTACCCTGATCAC |
| | | ATGAAGCAGCACGATTTCTTCAAGTCTGCCATGCCTGAGGGATA |
| | | CATTCAGGAGAGAACCATATTCTTCGAGGATGACGGGAACTACA |
| | | AGAGCAGGGCTGAGGTCAAGTTCGAAGGGGATACACTGGTAAAT |
| | | AGGATCGAGTTGACCGGCACAGATTTCAAGGAAGATGGAAACAT |
| | | CCTGGGCAATAAGATGGAGTACAACTATAACGCCCACAATGTCT |
| | | ACATAATGACCGATAAAGCTAAGAATGGCATAAAGGTTAATTTC |
| | | AAGATTAGGCACAACATAGAGGATGGGTCCGTGCAGCTTGCCGA |
| | | TCACTACCAGCAGAATACGCCCATAGGAGATGGACCTGTGTTGT |
| | | TACCTGATAACCACTATTTAAGTACTCAGAGTGCTTTATCAAAG |
| | | GACCCTAACGAAAAGAGGGATCACATGATCTACTTCGGGTTCGT |
| | | CACAGCGGCAGCAATAACCCACGGCATGGATGAGCTTTACAAGT |
| | | AAGGAGGGGGATTGTTACCGGTAAAAGAAGCTGAGGATAAACTC |
| | | AGCATAAATGATCCGCTTTTTGAGAGGCAGTGGCACTTGGTCAA |
| | | TCCAAGTTTTCCTGGCAGTGATATAAATGTTCTTGATCTGTGGT |
| | | ACAATAATATTACAGGCGCAGGGGTCGTGGCTGCCATTGTTGAT |
| | | GATGGCCTTGACTACGAAAATGAAGACTTGAAGGATAATTTTTG |
| | | CGCTGAAGGTTCTTGGGATTTCAACGACAATCGGGCCTGAGCAA |
| | | TAACTAGCAAAAAA |
| 29 | GE-328 | GCUAGTUATUGCUCAGGCCCGAUTGUCGTTGAAATCC |
| 30 | GE-348 | AGAGUTGCUAAAAUTGGGCAAAAGAUCATCATUAGAAGAGTTAC |
| | | A |
| 31 | GE-349 | GAGTTGCTAAAATTGGGCAAAAGATC |
| 32 | GE-351 | GCGGATCATTTATGCTGAGTTTATC |
| 33 | GE-352 | ACACTCTTTCCCTACACGACGCTCTTCCGATCTNGAGTTGCTAA |
| | | AATTGGGCAAAAGATC |
| 34 | GE-353 | ACACTCTTTCCCTACACGACGCTCTTCCGATCT NN |
| | | GAGTTGCTAAAATTGGGCAAAAGATC |
| 35 | GE-354 | ACACTCTTTCCCTACACGACGCTCTTCCGATCT NNN |
| | | GAGTTGCTAAAATTGGGCAAAAGATC |
| 36 | GE-355 | ACACTCTTTCCCTACACGACGCTCTTCCGATCT NNNN |
| | | GAGTTGCTAAAATTGGGCAAAAGATC |
| 37 | GE-356 | ACACTCTTTCCCTACACGACGCTCTTCCGATCT NNNNN |
| | | GAGTTGCTAAAATTGGGCAAAAGATC |
| 38 | GE-357 | ACACTCTTTCCCTACACGACGCTCTTCCGATCT NNNNNN |
| | | GAGTTGCTAAAATTGGGCAAAAGATC |
| 39 | GE-364 | GTGACTGGAGTTCAGACGTGTGCTCTTCCGATCT N |
| | | GCGGATCATTTATGCTGAGTTTATC |
| 40 | GE-365 | GTGACTGGAGTTCAGACGTGTGCTCTTCCGATCT NN |
| | | GCGGATCATTTATGCTGAGTTTATC |
| 41 | GE-366 | GTGACTGGAGTTCAGACGTGTGCTCTTCCGATCT NNN |
| | | GCGGATCATTTATGCTGAGTTTATC |
| 42 | GE-367 | GTGACTGGAGTTCAGACGTGTGCTCTTCCGATCT NNNN |
| | | GCGGATCATTTATGCTGAGTTTATC |
| 43 | GE-368 | GTGACTGGAGTTCAGACGTGTGCTCTTCCGATCT NNNNN |
| | | GCGGATCATTTATGCTGAGTTTATC |
| 44 | GE-369 | GTGACTGGAGTTCAGACGTGTGCTCTTCCGATCT NNNNNN |
| | | GCGGATCATTTATGCTGAGTTTATC |
| 45 | Sample | AATGATACGGCGACCACCGAGATCTACACATAGAGGCACACTCT |
| | index | TTCCCTACACGACGC |
| | primer GE- | |
| | 375 | |
| 46 | Sample | AATGATACGGCGACCACCGAGATCTACACCCTATCCTACACTCT |
| | index | TTCCCTACACGACGC |
| | primer | |
| | GE-376 | |
| 47 | Sample | CAAGCAGAAGACGGCATACGAGATTCCGGAGAGTGACTGGAGTT |
| | index | CAGACGTGT |
| | primer GE- | |
| | 383 | |
| 48 | Sample | CAAGCAGAAGACGGCATACGAGATCGCTCATTGTGACTGGAGTT |
| | index | CAGACGTGT |
| | primer GE- | |
| | 384 | |
| 49 | Primer GE- | ATGAAAGTGAGGAAATATATTACTTTATGCTTTTGGTGG |
| | 249 | |
| 50 | Primer GE- | GAGAAGCACTGAACCCCGTAACTTAAGGTG |
| | 173 | |
| 51 | Q45458 | MKKKLVLIDGNSVAYRAFFALPLLHNDKGIHTNAVYGFTMMLNK |
| | DNA | ILAEEQPTHLLVAFDAGKTTFRHETFQEYKGGRQQTPPELSEQF |
| | polymerase | PLLRELLKAYRIPAYELDHYEADDIIGTLAARAEQEGFEVKIIS |
| | I | GDRDLTQLASRHVTVDITKKGITDIEPYTPETVREKYGLTPEQI |
| | Geobacillus | VDLKGLMGDKSDNIPGVPGIGEKTAVKLLKQFGTVENVLASIDE |
| | stearotherm- | VKGEKLKENLRQHRDLALLSKQLASICRDAPVELSLDDIVYEGQ |
| | ophilus | DREKVIALFKELGFQSFLEKMAAPAAEGEKPLEEMEFAIVDVIT |
| | | EEMLADKAALVVEVMEENYHDAPIVGIALVNEHGRFFMRPETAL |
| | | ADSQFLAWLADETKKKSMFDAKRAVVALKWKGIELRGVAFDLLL |
| | | AAYLLNPAQDAGDIAAVAKMKQYEAVRSDEAVYGKGVKRSLPDE |
| | | QTLAEHLVRKAAAIWALEQPFMDDLRNNEQDQLLTKLEQPLAAI |
| | | LAEMEFTGVNVDTKRLEQMGSELAEQLRAIEQRIYELAGQEFNI |
| | | NSPKQLGVILFEKLQLPVLKKTKTGYSTSADVLEKLAPHHEIVE |
| | | NILHYRQLGKLQSTYIEGLLKVVRPDTGKVHTMFNQALTQTGRL |
| | | SSAEPNLQNIPIRLEEGRKIRQAFVPSEPDWLIFAADYSQIELR |
| | | VLAHIADDDNLIEAFQRDLDIHTKTAMDIFHVSEEEVTANMRRQ |
| | | AKAVNFGIVYGISDYGLAQNLNITRKEAAEFIERYFASFPGVKQ |
| | | YMENIVQEAKQKGYVTTLLHRRRYLPDITSRNFNVRSFAERTAM |
| | | NTPIQGSAADIIKKAMIDLAARLKEEQLQARLLLQVHDELILEA |
| | | PKEEIERLCELVPEVMEQAVTLRVPLKVDYHYGPTWYDAK |
| 52 | P00581 | MIVSDIEANALLESVTKFHCGVIYDYSTAEYVSYRPSDFGAYLD |
| | DNA- | ALEAEVARGGLIVFHNGHKYDVPALTKLAKLQLNREFHLPRENC |
| | directed | IDTLVLSRLIHSNLKDTDMGLLRSGKLPGKRFGSHALEAWGYRL |
| | DNA | GEMKGEYKDDFKRMLEEQGEEYVDGMEWWNFNEEMMDYNVQDVV |
| | polymerase | VTKALLEKLLSDKHYFPPEIDFTDVGYTTFWSESLEAVDIEHRA |
| | Escherichia | AWLLAKQERNGFPFDTKAIEELYVELAARRSELLRKLTETFGSW |
| | phage T7 | YQPKGGTEMFCHPRTGKPLPKYPRIKTPKVGGIFKKPKNKAQRE |
| | | GREPCELDTREYVAGAPYTPVEHVVFNPSSRDHIQKKLQEAGWV |
| | | PTKYTDKGAPVVDDEVLEGVRVDDPEKQAAIDLIKEYLMIQKRI |
| | | GQSAEGDKAWLRYVAEDGKIHGSVNPNGAVTGRATHAFPNLAQI |
| | | PGVRSPYGEQCRAAFGAEHHLDGITGKPWVQAGIDASGLELRCL |
| | | AHFMARFDNGEYAHEILNGDIHTKNQIAAELPTRDNAKTFIYGF |
| | | LYGAGDEKIGQIVGAGKERGKELKKKFLENTPAIAALRESIQQT |
| | | LVESSQWVAGEQQVKWKRRWIKGLDGRKVHVRSPHAALNTLLQS |
| | | AGALICKLWIIKTEEMLVEKGLKHGWDGDFAYMAWVHDEIQVGC |
| | | RTEEIAQVVIETAQEAMRWVGDHWNFRCLLDTEGKMGPNWAICH |
| 53 | P04415 | MKEFYISIETVGNNIVERYIDENGKERTREVEYLPTMFRHCKEE |
| | DNA- | SKYKDIYGKNCAPQKFPSMKDARDWMKRMEDIGLEALGMNDFKL |
| | directed | AYISDTYGSEIVYDRKFVRVANCDIEVTGDKFPDPMKAEYEIDA |
| | DNA | ITHYDSIDDRFYVFDLLNSMYGSVSKWDAKLAAKLDCEGGDEVP |
| | polymerase | QEILDRVIYMPFDNERDMLMEYINLWEQKRPAIFTGWNIEGFDV |
| | Enterobacteria | PYIMNRVKMILGERSMKRFSPIGRVKSKLIQNMYGSKEIYSIDG |
| | phage | VSILDYLDLYKKFAFTNLPSFSLESVAQHETKKGKLPYDGPINK |
| | T4 | LRETNHQRYISYNIIDVESVQAIDKIRGFIDLVLSMSYYAKMPF |
| | | SGVMSPIKTWDAIIFNSLKGEHKVIPQQGSHVKQSFPGAFVFEP |
| | | KPIARRYIMSFDLTSLYPSIIRQVNISPETIRGQFKVHPIHEYI |
| | | AGTAPKPSDEYSCSPNGWMYDKHQEGIIPKEIAKVFFQRKDWKK |
| | | KMFAEEMNAEAIKKIIMKGAGSCSTKPEVERYVKFSDDFLNELS |
| | | NYTESVLNSLIEECEKAATLANTNQLNRKILINSLYGALGNIHF |
| | | RYYDLRNATAITIFGQVGIQWIARKINEYLNKVCGTNDEDFIAA |
| | | GDTDSVYVCVDKVIEKVGLDRFKEQNDLVEFMNQFGKKKMEPMI |
| | | DVAYRELCDYMNNREHLMHMDREAISCPPLGSKGVGGFWKAKKR |
| | | YALNVYDMEDKRFAEPHLKIMGMETQQSSTPKAVQEALEESIRR |
| | | ILQEGEESVQEYYKNFEKEYRQLDYKVIAEVKTANDIAKYDDKG |
| | | WPGFKCPFHIRGVLTYRRAVSGLGVAPILDGNKVMVLPLREGNP |
| | | FGDKCIAWPSGTELPKEIRSDVLSWIDHSTLFQKSFVKPLAGMC |
| | | ESAGMDYEEKASLDFLFG |
| 54 | P19821 | MRGMLPLFEPKGRVLLVDGHHLAYRTFHALKGLTTSRGEPVQAV |
| | DNA | YGFAKSLLKALKEDGDAVIVVFDAKAPSFRHEAYGGYKAGRAPT |
| | polymerase | PEDFPRQLALIKELVDLLGLARLEVPGYEADDVLASLAKKAEKE |
| | I, | GYEVRILTADKDLYQLLSDRIHVLHPEGYLITPAWLWEKYGLRP |
| | thermostable | DQWADYRALTGDESDNLPGVKGIGEKTARKLLEEWGSLEALLKN |
| | Thermus | LDRLKPAIREKILAHMDDLKLSWDLAKVRTDLPLEVDFAKRREP |
| | aquaticus | DRERLRAFLERLEFGSLLHEFGLLESPKALEEAPWPPPEGAFVG |
| | | FVLSRKEPMWADLLALAAARGGRVHRAPEPYKALRDLKEARGLL |
| | | AKDLSVLALREGLGLPPGDDPMLLAYLLDPSNTTPEGVARRYGG |
| | | EWTEEAGERAALSERLFANLWGRLEGEERLLWLYREVERPLSAV |
| | | LAHMEATGVRLDVAYLRALSLEVAEEIARLEAEVFRLAGHPFNL |
| | | NSRDQLERVLFDELGLPAIGKTEKTGKRSTSAAVLEALREAHPI |
| | | VEKILQYRELTKLKSTYIDPLPDLIHPRTGRLHTRFNQTATATG |
| | | RLSSSDPNLQNIPVRTPLGQRIRRAFIAEEGWLLVALDYSQIEL |
| | | RVLAHLSGDENLIRVFQEGRDIHTETASWMFGVPREAVDPLMRR |
| | | AAKTINFGVLYGMSAHRLSQELAIPYEEAQAFIERYFQSFPKVR |
| | | AWIEKTLEEGRRRGYVETLFGRRRYVPDLEARVKSVREAAERMA |
| | | FNMPVQGTAADLMKLAMVKLFPRLEEMGARMLLQVHDELVLEAP |
| | | KERAEAVARLAKEVMEGVYPLAVPLEVEVGIGEDWLSAKE |
| 55 | P30317 | MILDTDYITKDGKPIIRIFKKENGEFKIELDPHFQPYIYALLKD |
| | DNA | DSAIEEIKAIKGERHGKTVRVLDAVKVRKKFLGREVEVWKLIFE |
| | polymerase | HPQDVPAMRGKIREHPAVVDIYEYDIPFAKRYLIDKGLIPMEGD |
| | Thermococcus | EELKLLAFDIETFYHEGDEFGKGEIIMISYADEEEARVITWKNI |
| | litoralis | DLPYVDVVSNEREMIKRFVQVVKEKDPDVIITYNGDNFDLPYLI |
| | | KRAEKLGVRLVLGRDKEHPEPKIQRMGDSFAVEIKGRIHFDLFP |
| | | WVRRTINLPTYTLEAVYEAVLGKTKSKLGAEEIAAIWETEESMK |
| | | KLAQYSMEDARATYELGKEFFPMEAELAKLIGQSVWDVSRSSTG |
| | | NLVEWYLLRVAYARNELAPNKPDEEEYKRRLRTTYLGGYVKEPE |
| | | KGLWENIIYLDFRSLYPSIIVTHNVSPDTLEKEGCKNYDVAPIV |
| | | GYRFCKDFPGFIPSILGDLIAMRQDIKKKMKSTIDPIEKKMLDY |
| | | RQRAIKLLANSILPNEWLPIIENGEIKFVKIGEFINSYMEKQKE |
| | | NVKTVENTEVLEVNNLFAFSFNKKIKESEVKKVKALIRHKYKGK |
| | | AYEIQLSSGRKINITAGHSLFTVRNGEIKEVSGDGIKEGDLIVA |
| | | PKKIKLNEKGVSINIPELISDLSEEETADIVMTISAKGRKNFFK |
| | | GMLRTLRWMFGEENRRIRTFNRYLFHLEKLGLIKLLPRGYEVTD |
| | | WERLKKYKQLYEKLAGSVKYNGNKREYLVMFNEIKDFISYFPQK |
| | | ELEEWKIGTLNGFRTNCILKVDEDFGKLLGYYVSEGYAGAQKNK |
| | | TGGISYSVKLYNEDPNVLESMKNVAEKFFGKVRVDRNCVSISKK |
| | | MAYLVMKCLCGALAENKRIPSVILTSPEPVRWSFLEAYFTGDGD |
| | | IHPSKRFRLSTKSELLANQLVFLLNSLGISSVKIGFDSGVYRVY |
| | | INEDLQFPQTSREKNTYYSNLIPKEILRDVFGKEFQKNMTFKKF |
| | | KELVDSGKLNREKAKLLEFFINGDIVLDRVKSVKEKDYEGYVYD |
| | | LSVEDNENFLVGFGLLYAHNSYYGYMGYPKARWYSKECAESVTA |
| | | WGRHYIEMTIREIEEKFGFKVLYADSVSGESEIIIRQNGKIRFV |
| | | KIKDLFSKVDYSIGEKEYCILEGVEALTLDDDGKLVWKPVPYVM |
| | | RHRANKRMFRIWLTNSWYIDVTEDHSLIGYLNTSKTKTAKKIGE |
| | | RLKEVKPFELGKAVKSLICPNAPLKDENTKTSEIAVKFWELVGL |
| | | IVGDGNWGGDSRWAEYYLGLSTGKDAEEIKQKLLEPLKTYGVIS |
| | | NYYPKNEKGDFNILAKSLVKFMKRHFKDEKGRRKIPEFMYELPV |
| | | TYIEAFLRGLFSADGTVTIRKGVPEIRLTNIDADFLREVRKLLW |
| | | IVGISNSIFAETTPNRYNGVSTGTYSKHLRIKNKWRFAERIGFL |
| | | IERKQKRLLEHLKSARVKRNTIDFGFDLVHVKKVEEIPYEGYVY |
| | | DIEVEETHRFFANNILVHNTDGFYATIPGEKPELIKKKAKEFLN |
| | | YINSKLPGLLELEYEGFYLRGFFVTKKRYAVIDEEGRITTRGLE |
| | | VVRRDWSEIAKETQAKVLEAILKEGSVEKAVEVVRDVVEKIAKY |
| | | RVPLEKLVIHEQITRDLKDYKAIGPHVAIAKRLAARGIKVKPGT |
| | | IISYIVLKGSGKISDRVILLTEYDPRKHKYDPDYYIENQVLPAV |
| | | LRILEAFGYRKEDLRYQSSKQTGLDAWLKR |
| 56 | Chain A, | VISYDNYVTILDEETLKAWIAKLEKAPVFAFDTETDSLDNISAN |
| | DNA | LVGLSFAIEPGVAAYIPVAHDYLDAPDQISRERALELLKPLLED |
| | Polymerase | EKALKVGQNLKYDRGILANYGIELRGIAFDTMLESYILNSVAGR |
| | I Klenow | HDMDSLAERWLKHKTITFEEIAGKGKNQLTFNQIALEEAGRYAA |
| | Fragment | EDADVTLQLHLKMWPDLQKHKGPLNVFENIEMPLVPVLSRIERN |
| | PDB: | GVKIDPKVLHNHSEELTLRLAELEKKAHEIAGEEFNLSSTKQLQ |
| | 1KFD_A | TILFEKQGIKPLKKTPGGAPSTSEEVLEELALDYPLPKVILEYR |
| | | GLAKLKSTYTDKLPLMINPKTGRVHTSYHQAVTATGRLSSTDPN |
| | | LQNIPVRNEEGRRIRQAFIAPEDYVIVSADYSQIELRIMAHLSR |
| | | DKGLLTAFAEGKDIHRATAAEVFGLPLETVTSEQRRSAKAINFG |
| | | LIYGMSAFGLARQLNIPRKEAQKYMDLYFERYPGVLEYMERTRA |
| | | QAKEQGYVETLDGRRLYLPDIKSSNGARRAAAERAAINAPMQGT |
| | | AADIIKRAMIAVDAWLQAEQPRVRMIMQVHDELVFEVHKDDVDA |
| | | VAKQIHQLMENCTRLDVPLLVEVGSGENWDQAH |
| 57 | P03680 | MKHMPRKMYSCDFETTTKVEDCRVWAYGYMNIEDHSEYKIGNSL |
| | DNA | DEFMAWVLKVQADLYFHNLKFDGAFIINWLERNGFKWSADGLPN |
| | polymerase | TYNTIISRMGQWYMIDICLGYKGKRKIHTVIYDSLKKLPFPVKK |
| | Bacillus | IAKDFKLTVLKGDIDYHKERPVGYKITPEEYAYIKNDIQIIAEA |
| | phage | LLIQFKQGLDRMTAGSDSLKGFKDIITTKKFKKVFPTLSLGLDK |
| | phi29 | EVRYAYRGGFTWLNDRFKEKEIGEGMVFDVNSLYPAQMYSRLLP |
| | | YGEPIVFEGKYVWDEDYPLHIQHIRCEFELKEGYIPTIQIKRSR |
| | | FYKGNEYLKSSGGEIADLWLSNVDLELMKEHYDLYNVEYISGLK |
| | | FKATTGLFKDFIDKWTYIKTTSEGAIKQLAKLMLNSLYGKFASN |
| | | PDVTGKVPYLKENGALGFRLGEEETKDPVYTPMGVFITAWARYT |
| | | TITAAQACYDRIIYCDTDSIHLTGTEIPDVIKDIVDPKKLGYWA |
| | | HESTFKRAKYLRQKTYIQDIYMKEVDGKLVEGSPDDYTDIKFSV |
| | | KCAGMTDKIKKEVTFENFKVGFSRKMKPKPVQVPGGVVLVDDTF |
| | | TIK |
| 58 | O75417 | GFKDNSPISDTSFSLQLSQDGLQLTPASSSSESLSIIDVASDQN |
| | DNA | LFQTFIKEWRCKKRFSISLACEKIRSLTSSKTATIGSRFKQASS |
| | polymerase | PQEIPIRDDGFPIKGCDDTLVVGLAVCWGGRDAYYFSLQKEQKH |
| | theta Homo | SEISASLVPPSLDPSLTLKDRMWYLQSCLRKESDKECSVVIYDF |
| | sapiens | IQSYKILLLSCGISLEQSYEDPKVACWLLDPDSQEPTLHSIVTS |
| | | FLPHELPLLEGMETSQGIQSLGLNAGSEHSGRYRASVESILIFN |
| | | SMNQLNSLLQKENLQDVFRKVEMPSQYCLALLELNGIGFSTAEC |
| | | ESQKHIMQAKLDAIETQAYQLAGHSFSFTSSDDIAEVLFLELKL |
| | | PPNREMKNQGSKKTLGSTRRGIDNGRKLRLGRQFSTSKDVLNKL |
| | | KALHPLPGLILEWRRITNAITKVVFPLQREKCLNPFLGMERIYP |
| | | VSQSHTATGRITFTEPNIQNVPRDFEIKMPTLVGESPPSQAVGK |
| | | GLLPMGRGKYKKGFSVNPRCQAQMEERAADRGMPFSISMRHAFV |
| | | PFPGGSILAADYSQLELRILAHLSHDRRLIQVLNTGADVFRSIA |
| | | AEWKMIEPESVGDDLRQQAKQICYGIIYGMGAKSLGEQMGIKEN |
| | | DAACYIDSFKSRYTGINQFMTETVKNCKRDGFVQTILGRRRYLP |
| | | GIKDNNPYRKAHAERQAINTIVQGSAADIVKIATVNIQKQLETF |
| | | HSTFKSHGHREGMLQSDQTGLSRKRKLQGMFCPIRGGFFILQLH |
| | | DELLYEVAEEDVVQVAQIVKNEMESAVKLSVKLKVKVKIGASWG |
| | | ELKDFDV |

### EXAMPLES

### Example 1.—Testing Functionality of DNA Polymerase-Cas9-Insert Template Fusion Complexes

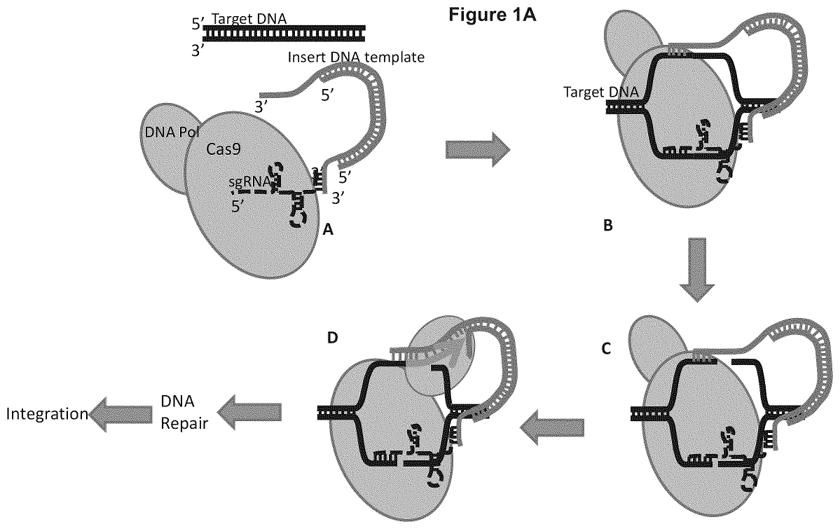

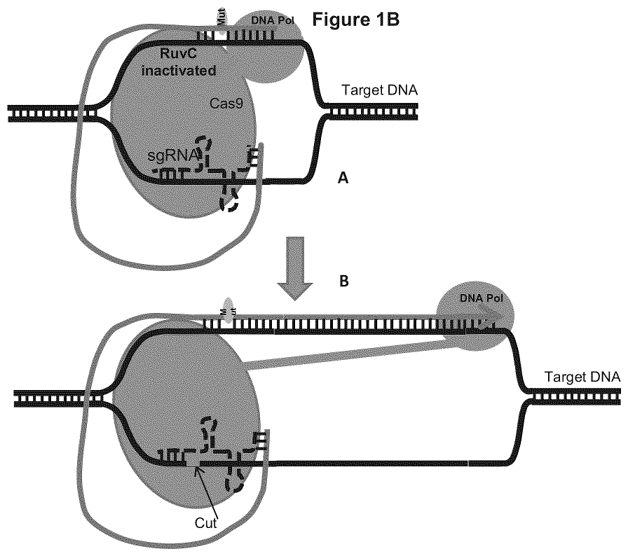

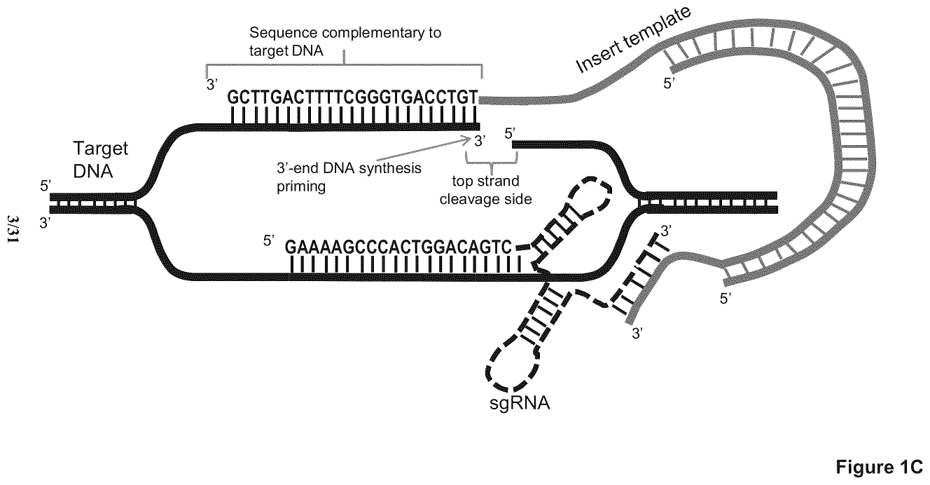

The ability of DNA polymerase-Cas9-insert DNA complexes (see FIG. 1) to insert DNA at a desired was determined to depend on three steps: (1) the Cas9, sgRNA, and insert DNA forming a stable complex (see FIG. 1A-B); (2) the Cas9-sgRNA complex binding efficiently to the sgRNA target site and support inserting DNA annealing to the target (see FIG. 1A-B); and (3) the polymerase being able to “pick up” and join the cleaved template at the target site to incorporate the insert DNA onto the cleaved target DNA strand (see FIG. 1A-B). Accordingly, in vitro reactions were performed to test the efficiency of each of these three steps (see Examples 2-4 below).

FIG. 1C illustrates in detail one proposed arrangement of the hybridization site from the insert DNA, the cut site of the target DNA, the insert template, the hybridization site between the sgRNA and the insert template, and the hybridization of the “seed” region of the sgRNA to the target DNA. In this arrangement, the sequence complementary to the target DNA site incorporated into the DNA insert template is included on the 3′ end of one strand of a double-stranded insert template, and is complementary to about 23 nucleotides immediately preceding the DNA cleavage site by the endonuclease or Cas9 on the top (5′-3′ oriented) DNA strand near the cleavage site.

### Example 2.—CRISPR sgRNAs Accommodate Hybridization Domain to DNA Inserts

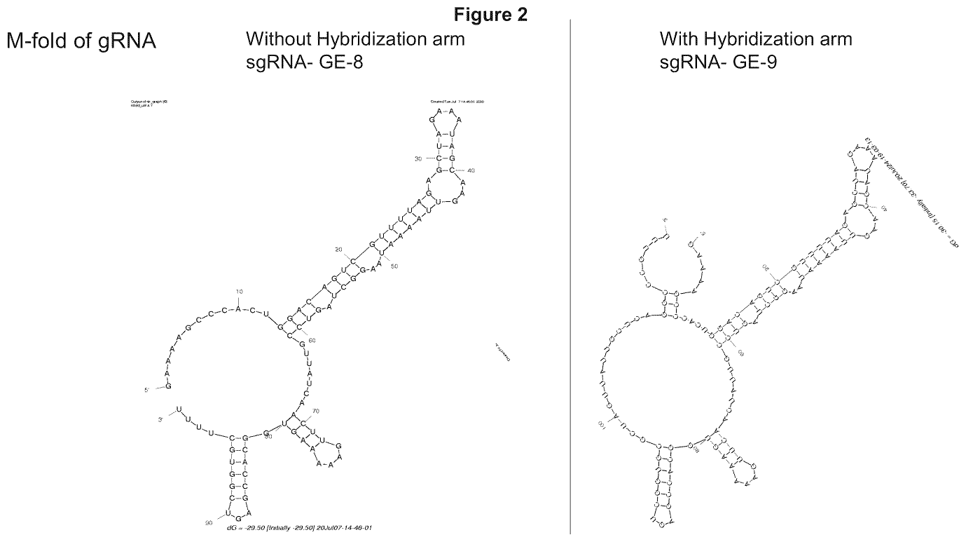

Towards to goal of generating a polymerase-Cas-sgRNA-insert DNA complex as shown in FIG. 1A, the ability of CRISPR single-guide RNAs (sgRNAs) to accommodate a 3′ hybridization domain for linking to a cargo insert DNA was first tested to verify the extra 3′ portion does not interfere with function of the Cas endonuclease. Guide RNAs directed against a region of lambda DNA, either with (GE-9, SEQ ID NO: 9, see FIG. 2, left panel) or without (GE-8, SEQ ID NO:8, see FIG. 2, right panel) were synthesized and tested with Cas9 in vitro for their ability to cleave lambda DNA.

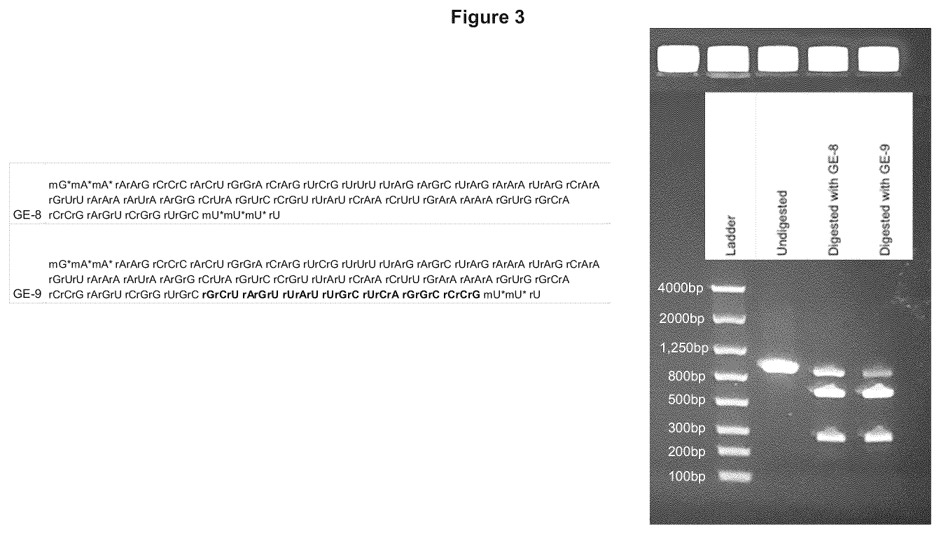

For these tests, a 1000 bp region of lambda DNA containing the sgRNA hybridization site was amplified using primers GE-4 and GE-5 (“GE4-5 Lambda DNA”). Next, Cas 9 ribonucleoprotein complexes were formed by combining 500 nM sgRNA GE-8 or GE-9 and 1 μM Cas9 (SpCas9 or Cas9 Nuclease, S. pyogenes purchased from New England Biolabs) in reaction buffer NEB 3.1 (New England Biolabs) for 10 min at 25C. After pre-incubation a cleavage reaction was then initiated by mixing 3 nM purified GE4-5 Lambda with each of the Cas9 ribonucleoprotein complexes generated above in NEB3.1 cleavage buffer at 37C for 15 min. After the incubation, the reaction was cleaned using SPRI beads (Beckman coulter) and analyzed on an agarose gel for efficiency of cleavage between GE-8 and GE-9 sgRNAs (FIG. 3). As the abundance of molecular weight bands smaller than 1000 bp was observed to be roughly equal between lanes 3 and 4 of the reactions, it was determined that addition of the 3′ hybridization domain in GE-9 had no meaningful effect on the ability of the sgRNA to cleave target DNA.

### Example 3.—Polymerase is Able to Repair Cleaved DNA at Insert Site to Incorporate Donor DNA

Next, the ability of polymerases to “pick up” insert DNA and insert it at a Cas9 cleavage site (see step C on FIG. 1A) was tested. For this experiment, lambda DNA amplified with primers GE-15 and GE-16 (“GE15-16 insert DNA”) bearing a 3′ end capable of hybridizing near the Cas9 cleavage site was used as an insert DNA, and the ability of a polymerase to “pick up” and extend a strand of GE6-7 Lambda DNA cleaved at the GE-8 site to insert the GE5-7 cargo sequence was tested.

FIG. 1C illustrates in detail one proposed arrangement of the hybridization site from the insert DNA, the cut site of the target DNA, the insert template, the hybridization site between the sgRNA and the insert template, and the hybridization of the “seed” region of the sgRNA to the target DNA. In this arrangement, the sequence complementary to the target DNA site incorporated into the DNA insert template is included on the 3′ end of one strand of a double-stranded insert template, and is complementary to about 23 nucleotides immediately preceding the DNA cleavage site by the endonuclease or Cas9 on the top (5′-3′ oriented) DNA strand near the cleavage site.

First, ribonucleoprotein complexes between GE-9 sgRNA and Cas9 (SpCas9 or Cas9 Nuclease, S. pyogenes purchased from New England Biolabs) were formed by combining 50 nM sgRNA GE-9 and 40 nM Cas9 (SpCas9 or Cas9 Nuclease, S. pyogenes purchased from New England Biolabs) in a reaction buffer NEB 3.1 (New England Biolabs) for 10 min at 25C. After incubation, a combined cleavage/extension reaction was initiated by combining: 1 ng/ul GE6-7 amplified Lambda, 400 nM GE15-16 insert DNA, 0.8 mM dNTPs and 1.2 U/ul Bst 2.0 (NEB). Four control reactions were simultaneously assembled, each missing a single component (No Cas9, no DNA polymerase, no sgRNA, and no insert) to demonstrate that any generated fusion product between the GE6-7 and GE15-16 sequences was dependent on all the components. The reactions were incubated sequentially: 15 min at 37C, 5 min at 55C, 5min at 60C and stopped. The cycled reactions were then treated with Exol (NEB) to remove excess DNA primers, the exonuclease was heat killed, the reaction was cleaned using SPRI beads (Beckman coulter), and the reaction was analyzed by qPCR using two primers (one specific for a region of GE6-7—primer GE20; and one specific for a region of GE15-16—primer GE-6) to detect the formation of a hybrid sequence by cleavage and extension.

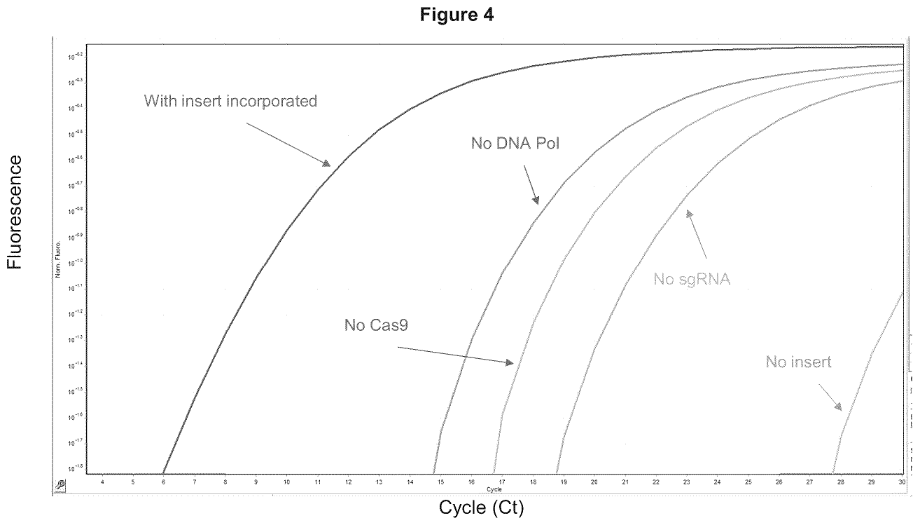

The QPCR Ct traces (lower Ct corresponding to larger amount of product) in FIG. 4 demonstrate that a DNA polymerase (Bst2.0) is able to pick up a Cas9 cleaved DNA strand and incorporate an insert DNA template at the cleavage site, as evidenced by the estimated ˜500× difference in product between the reaction with all the components (leftmost curve in FIG. 4 Ct trace) and the highest control reaction (next-to-leftmost curve in FIG. 4 Ct trace).

### Example 4.—Testing Ability of Polymerase to Incorporate Inserts of Various Size to Cas9-Cleaved DNA Strand

Next, the ability of polymerases to incorporate templates of increasing length onto Cas9 (SpCas9 or Cas9 Nuclease, S. pyogenes purchased from New England Biolabs) cleaved DNA strands (as shown in Example 2) was tested. Inserts of 50 bp (annealed oligos GE-15 and GE-16), 200 bp (annealed oligos GE-10 and GE11), 500 bp (GE-1 and GE-13 PCR amplified PUC18 DNA digested by Thermolabile USER 11 from NEB to generate 3′-overhangs), and 2000 bp (GE-1 and GE-14 PCR amplified PUC18 DNA digested by Thermolabile USER 11 from NEB to generate 3′-overhangs) were used in individual cleavage/extension reactions of otherwise similar composition to Example 2 to test the efficiency of hybrid product formation between the Cas9 cleaved DNA strands. The reactions were then amplified by PCR using two primers, one primer common to the Cas9-cleaved lambda DNA strand (GE-20) and one primer specific to and proximal to the end of each of the inserts (GE-6 for 50bp and 200 bp inserts, GE-2 for the 500bp inserts, and GE-3 for the 2000bp insert) to amplify any hybrid extension products between the Cas9-cleaved DNA and inserts. The reactions were then analyzed by agarose gel electrophoresis.

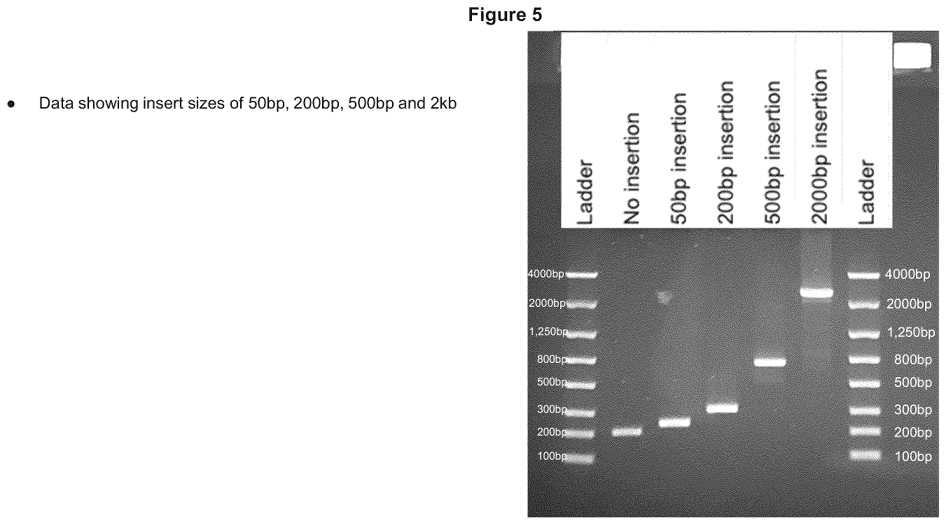

FIG. 5 lanes 3-6 demonstrate that the Cas9 cleavage/extension reaction is efficient from 50bp up to 2,000 bp, indicating that the extension/fusion reaction catalyzed by Cas9 and polymerase is capable of accommodating even quite large (2,000 bp) DNA inserts. Such reactions performed in vivo would dramatically increase the efficiency of large insertions at genomic sites.

### Example 4A.—Testing Ability of Insert DNA Annealed to sgRNA to be Incorporated at Cas9 Cleaved Site in DNA

Finally, the ability of the Cas9-sgRNA complex with insert DNA annealed to the end of the sgRNA (see intermediate C to intermediate D in FIG. 1A) rather than merely inserted in the cleavage/extension reaction was tested. A cleavage/reaction scheme similar to Example 2 was used, an additional pre-cleavage/extension step was performed to anneal the insert DNA to the end of the sgRNA.

First, reactions composed of 50nM sgRNA GE-8 or GE-9 (sgRNA GE-8 and GE-9 are similar exempt GE-9 includes sequence annealing to insert DNA) and 40 nM Cas9 (NEB) were assembled in reaction buffer NEB 3.1 (New England Biolabs) for 10 min at 25C, to form sgRNA/Cas9 complexes. Next, 400 nM GE-15-16 was added and incubated 10min at 25C to allow annealing between sgRNA and GE 15-16 to form Cas9-sgRNA-GE15-16 insert nucleic acid complexes.

Next, the Cas9-sgRNA-GE15-16 nucleic acid complexes were then mixed with 0.8 mM dNTPs and 1.2 U/ul Bst 2.0 (NEB) and the reactions were incubated sequentially: 15 min at 37C, 5 min at 55C, 5 min at 60C and stopped. After Exo I treatment, Exo I inactivation, and SPRI purification, the reactions were analyzed by QPCR to assess formation of hybrid products between Cas9 cleaved DNA and the inserts. As in the previous examples, the QPCR reactions utilized two primers: one binding to a site on the Cas9-cleaved lambda DNA (GE-20) and one binding near the end of the full insert sequence (GE-6).

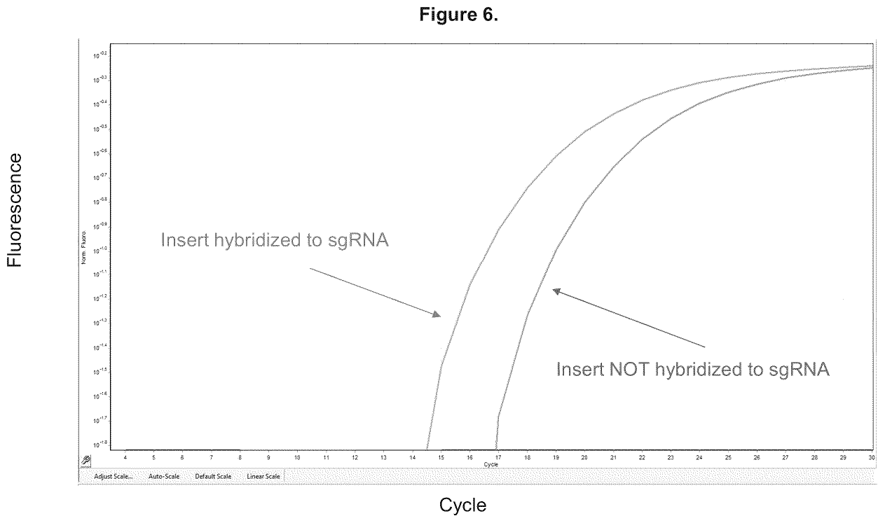

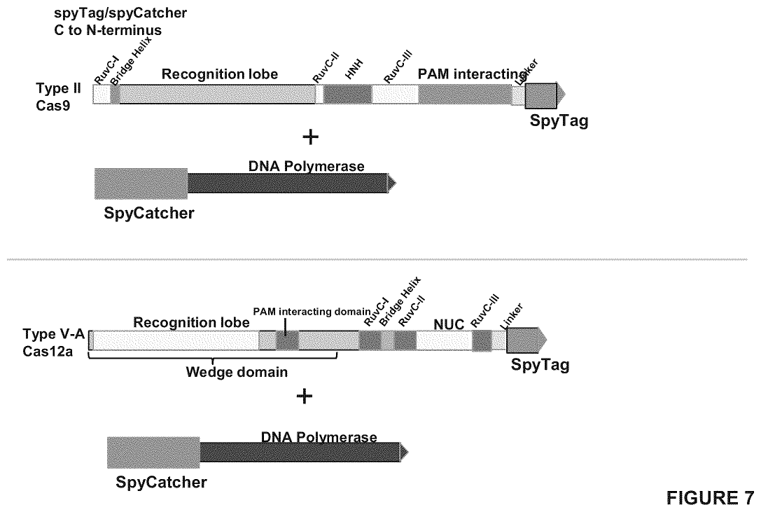

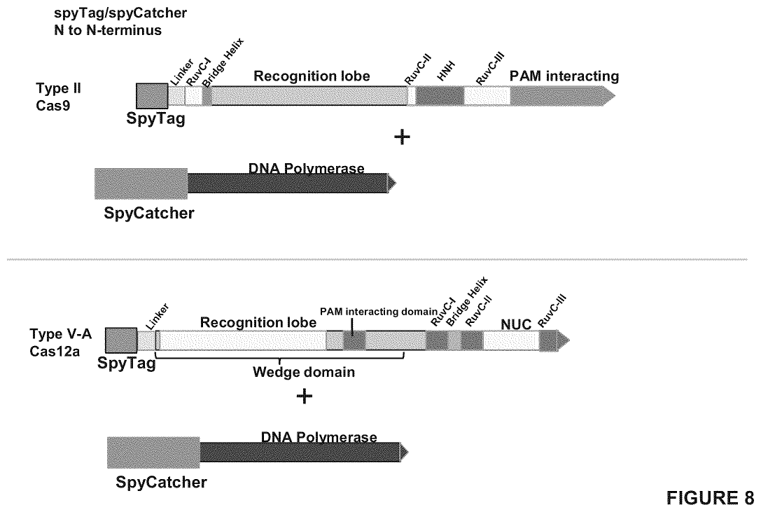

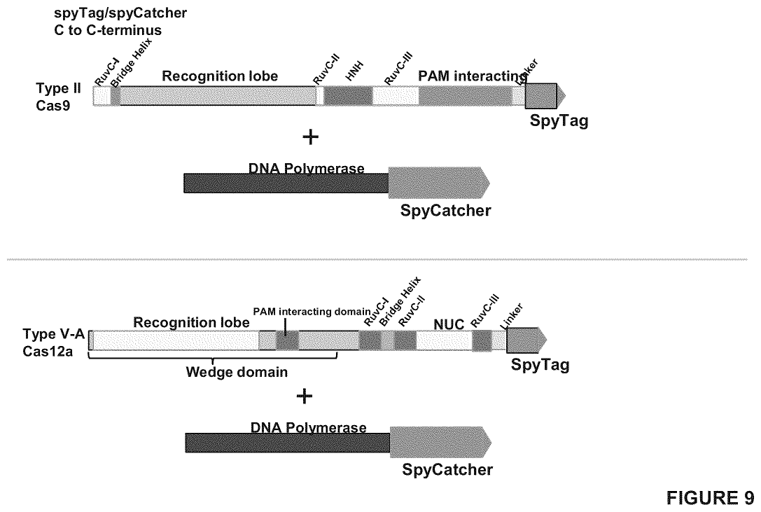

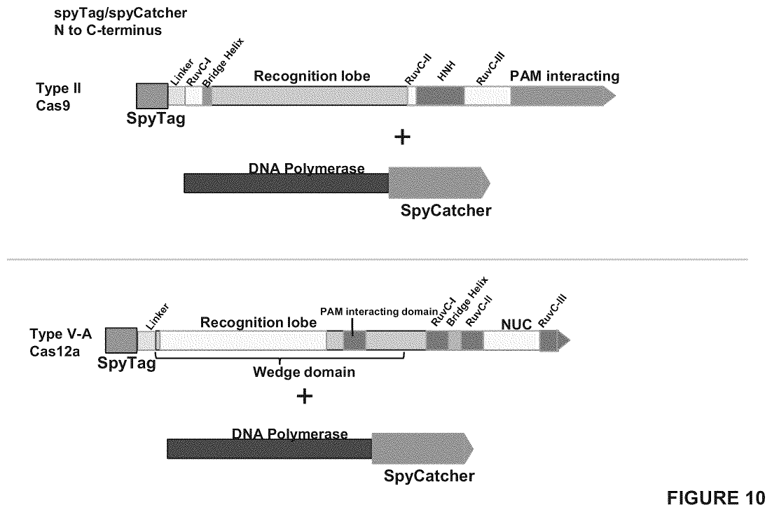

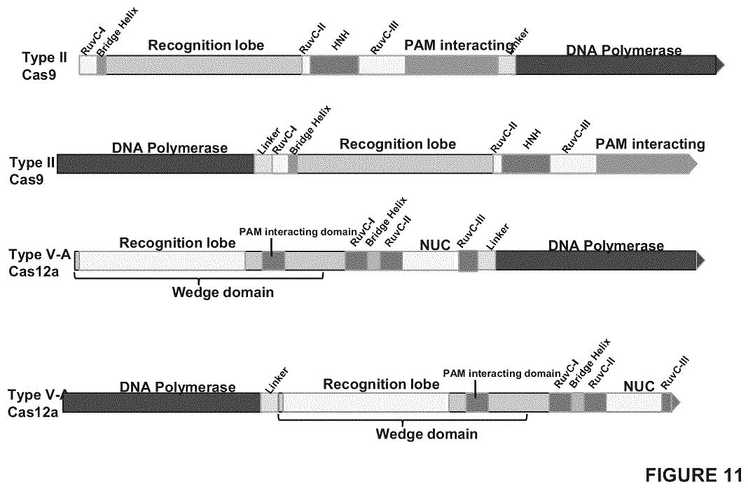

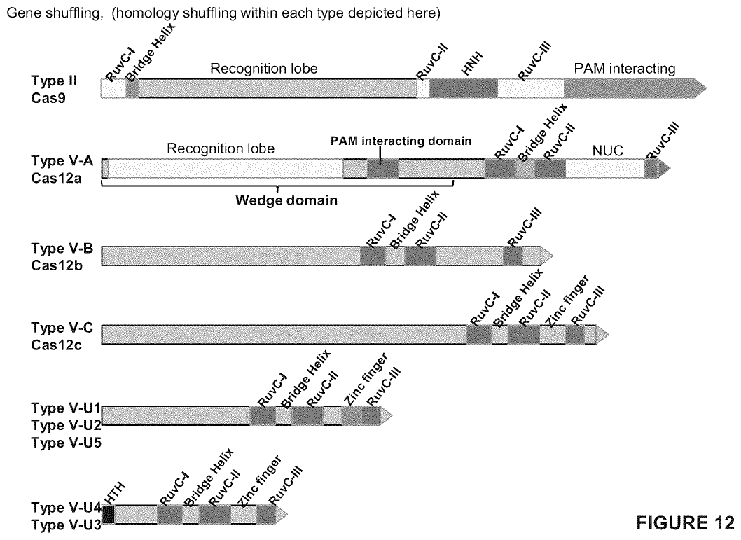

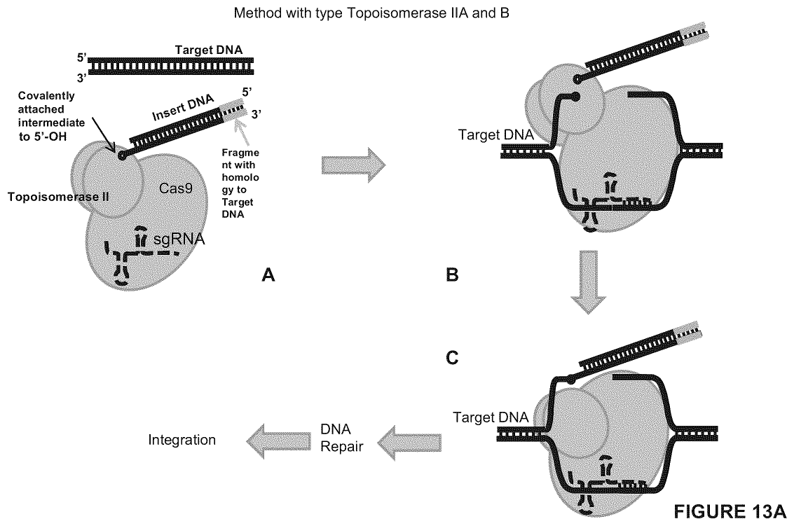

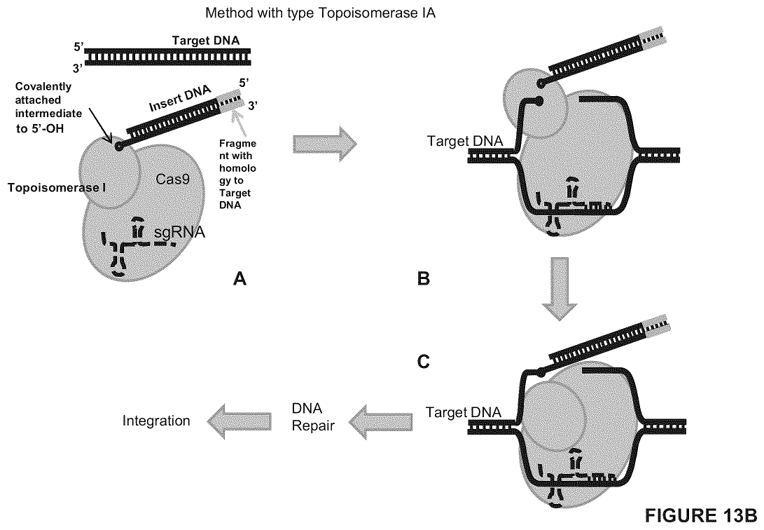

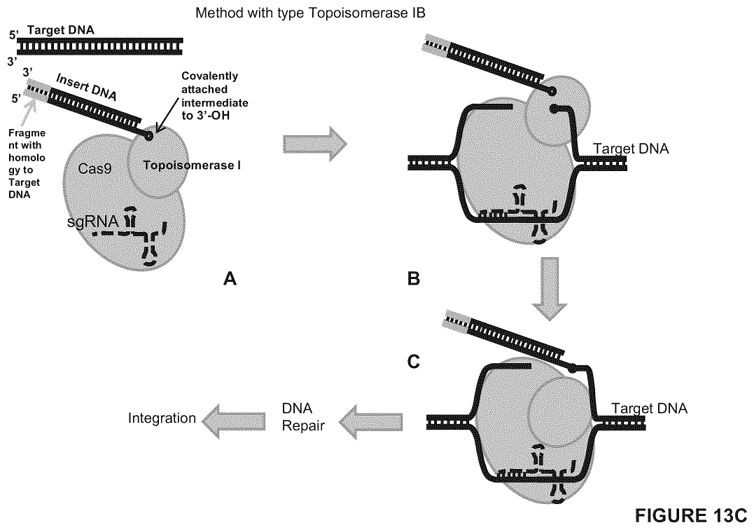

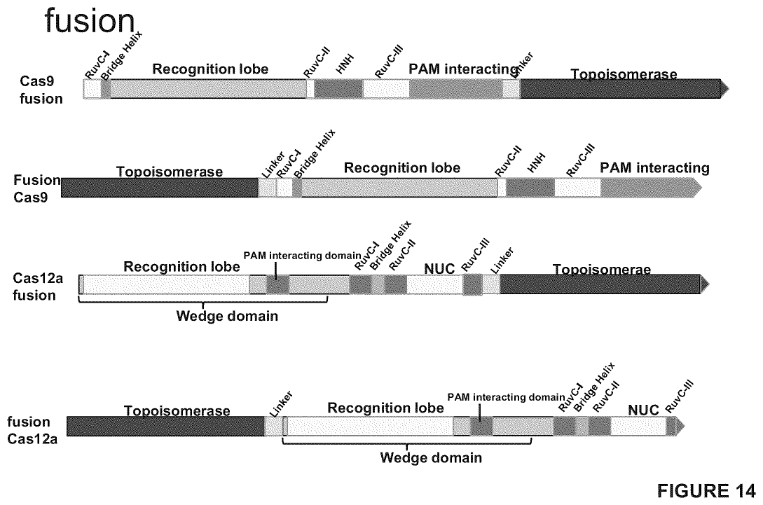

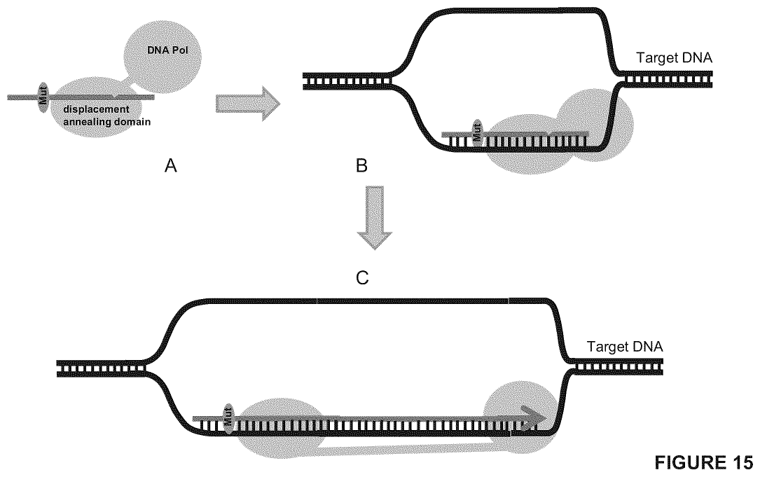

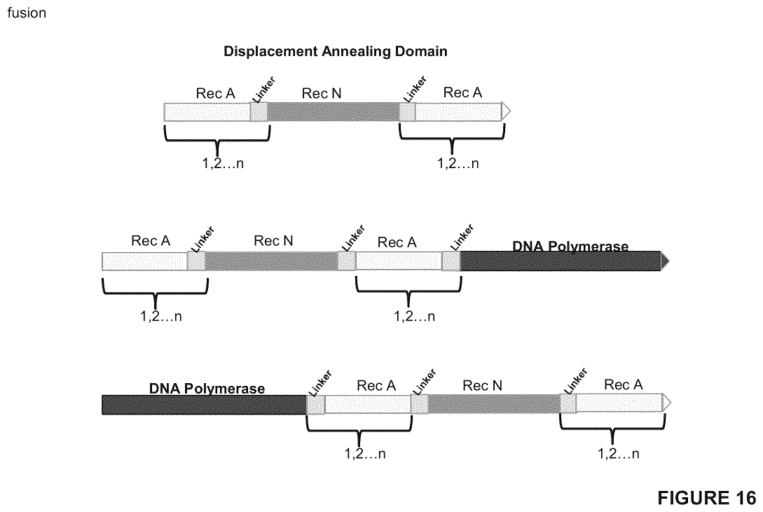

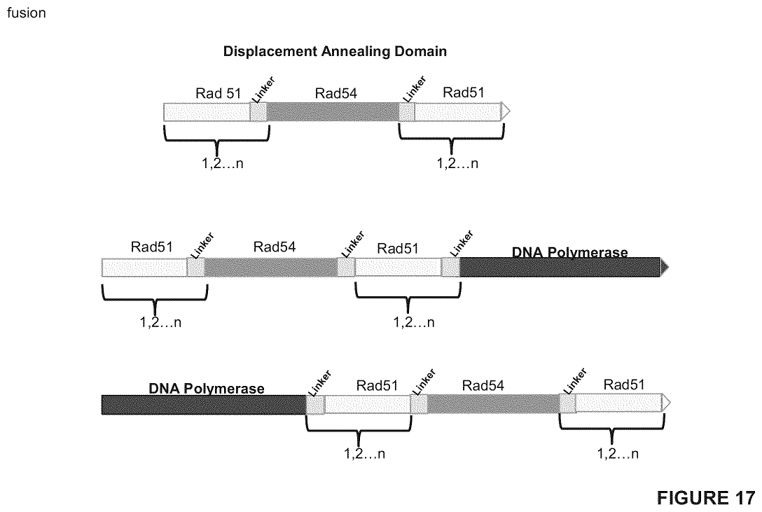

The results of the QPCR reactions are shown in FIG. 6. As demonstrated by the increased cycle time of the leftmost curve versus the rightmost curve, the Cas-sgRNA complex with annealed GE15-16 was both: (a) able to cleave and extend DNA to incorporate the insert, and (b) able to incorporate insert more efficiently (e.g. with a higher yield) than the comparable complex where insert was not annealed to the sgRNA.

### Example 5.—Assembly of Cas/Polymerase Fusions

### Version 1 Recombinant Fusion Expression

Endonuclease (e.g. Cas9) and the desired polymerase are provided fused in frame in a single open reading frame and are expressed in E. coli or another suitable host organism bearing an affinity tag. Suitable polypeptide linker sequences such as (GGGS)ₙ (SEQ ID NO: 67) are optionally encoded between the two enzymes in the expression construct. Induction of expression of the fusion protein according to standard recombinant expression procedures and affinity purification yields the endonuclease/polymerase fusion.

### Version 2—Linkage After Recombinant Expression

Endonuclease (e.g. Cas9) and the polymerase are expressed separately and subsequently are conjugated using SpyTag/SpyCather method. In this example Cas9 ORF is in-frame fused on its C-Terminus with AA sequence coding a SpyCatcher sequence (e.g. SEQ ID NO: 18) where the polymerase ORF on its N-terminus is in-frame fused with AA sequence coding for a SpyTag protein (e.g. SEQ ID NO: 19). Recombinant Expression of Proteins (general to either Version 1 or Version 2 above)

In either method above, ORFs are cloned into expression vector pET-45b. This vector includes a T7 polymerase promoter and the ORFs are fused with His-tag at the N-terminus. The expression constructs are transformed into E. coli BL21 (DE3). Before expression, a pre-culture is prepared in 2 ml LB with 100 μM carbenicillin and grown overnight for about 8 to 12 hours at 30C temperature. After about 8 to 12 h, 500 μL of the pre-culture was transferred to 25 mL of an auto-induction expression media, Overnight Express TB (Novagen), and the inoculated medium is shaker-incubated at room temperature for 30 hours to 48 hours. Cells are harvested by centrifugation at 4000 rpm for 15 min at 4-10C The biomass-pellet is frozen at −20 C for a minimum of 1 hour.

About 50-100 μl of pellet is melted and re-suspended in 0.5 mL lysis buffer and incubated for 30 minutes at room temperature (lysis buffer composition: 1×BugBuster, 100 mM Sodium Phosphate, 0.1% Tween, 2.5 mM TCEP, 3-5 μL, Protease inhibitor mix (Roche), 50 micro g lysozyme, 0.5 μL DNaseI (2,000 units/ml, from NEB)). After incubation, the lysate is mixed with an equal volume (0.5 mL) of His-binding buffer composed of 50 mM Sodium Phosphate pH 7.7, 1.5M Sodium Chloride, 2.5 mM TCEP, 0.1% Tween, 0.03% Triton X-100, and 10 mM Imidazole and the lysate is incubated at room temperature for about 15-30 minutes. After incubation, the lysate is centrifuged at 15000 rpm in a refrigerated microcentrifuge for about 15 min at a temperature from about 8 C. The resultant pellet is then mixed with 250 μL of His-Affinity Gel (His-Spin Protein Miniprep by Zymo Research) according to the manufacturer's protocol. After the binding step, the His-Affinity Gel is washed three times with washing buffer composed of 50 mM Sodium Phosphate pH 7.7, 750 mM Sodium Chloride, 0.1% Tween, 0.03% Triton X-100, 2.5 mM TCEP, and 50 mM Imidazole. The expressed protein is eluted with 100 to 250 μL of elution buffer composed of 50 mM Sodium Phosphate pH 7.7, 300 mM Sodium Chloride, 2.5 mM TCEP, 0.1% Tween, and 250 mM Imidazole.

After purification, purity of eluted samples is analyzed with 4-12% SDS polyacrylamide gel electrophoresis and stained using AcquaStain Protein Gel Stain (BulldogBio).

### Example 6.—Genome Editing in Cells (Prophetic)

A plasmid encoding a polymerase-endonuclease (e.g. Cas9-polymerase) fusion is co-transfected into cells alongside a plasmid encoding sgRNA targeting the desired genomic site and an insert DNA designed according to Example 1. The cells are incubated a period of time to allow for expression of the fusion protein and the sgRNA, and analysis of the genomic DNA is performed by a suitable technique to detect insertion of the insert DNA at the cut site specified by the sgRNA.

### Example 7.—Genome Editing in Yeast

Having observed successful results using polymerase-endonuclease fusions according to the invention in e.g. Example 4 or 4A, the system was tested for ability to edit genomic loci in yeast (Saccharomyces cerevisiae strain EBY100).

### Experiment Design

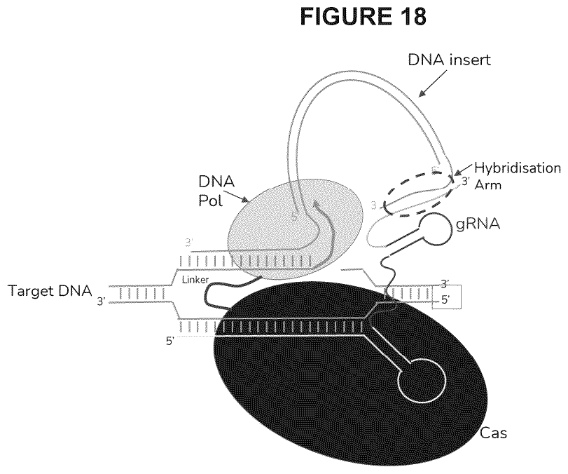

FIG. 18 illustrates the arrangement of the system used for yeast genome editing (particularly the insertion of a DNA insert at a specific locus). The system comprises a fusion protein, a gRNA, and a DNA insert. The fusion protein comprises an endonuclease (e.g. a Cas effector) fused to a DNA polymerase (which can have strand displacement, high processivity & high-fidelity properties). The fusion protein can comprise, e.g. the SpCas9 sequence from SEQ ID NO: 14 and the Bst2.0 sequence from SEQ ID NO:16. The gRNA targets the desired insertion site in genomic DNA, is capable of binding to the Cas effector, and has an extended 3′ arm for DNA insert. The 3′ arm of the gRNA allows for hybridization to a DNA insert, which can range in size from e.g. about 50 nucleotides to about 5000 nucleotides in length. The DNA insert additionally comprises a 3′ single-stranded region capable of hybridizing to one of the DNA strands at the site targeted by the gRNA. Once the endonuclease specifically recognizes and cleaves the site targeted by the gRNA and cleaves the target DNA, the resulting 3′ end liberated from the DNA can hybridize to the insert DNA, which can then be extended by the DNA polymerase. The resulting product extended by the DNA polymerase is covalently attached to the target DNA on one end and has dsDNA flap. Without wishing to be bound by theory, it is understood that homologous recombination on the dsDNA flap side of the insert can allow integration of the whole DNA insert in a precise and efficient manner.

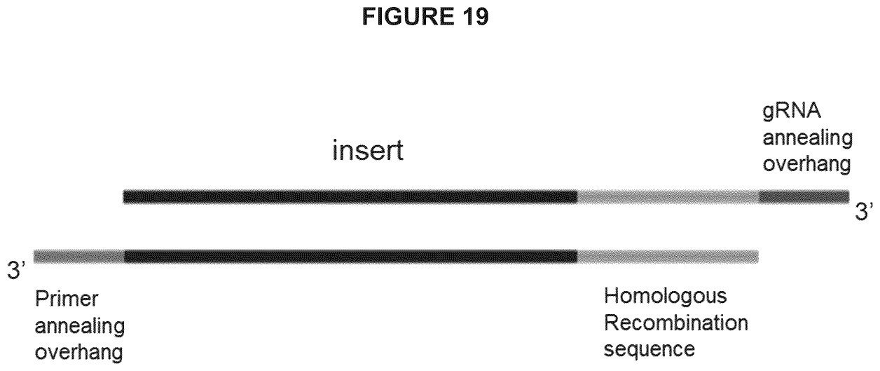

FIG. 19 depicts a schematic illustrating the design of the DNA insert used for this experiment. The insert template was a dsDNA with two 3′-single-stranded overhangs. One of the overhangs was configured to hybridize to gRNA adapter sequence. The second overhang was configured to anneal to the genomic target sequence near the Cas endonuclease cleavage site. After annealing of the overhang to the released by Cas endonuclease target 3′-end, the target DNA was configured to serve as an extension primer to produce an extended product by the DNA polymerase part of the endonuclease-DNA polymerase fusion.

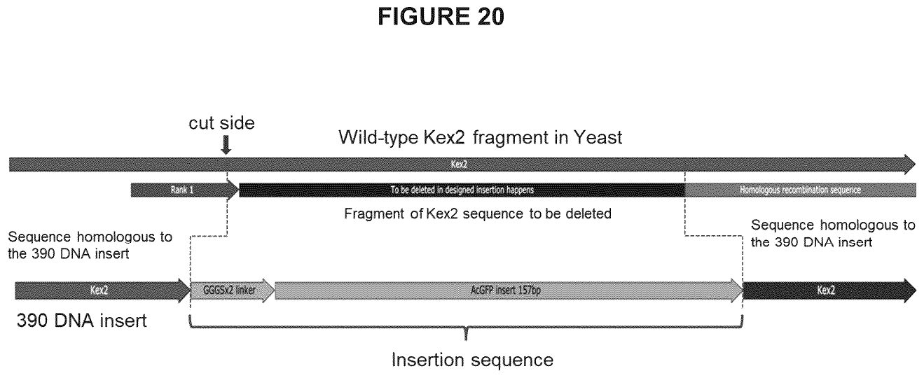

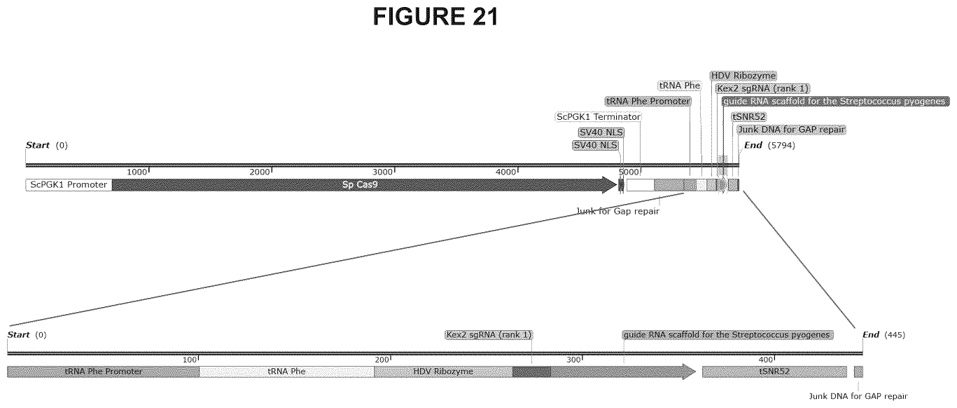

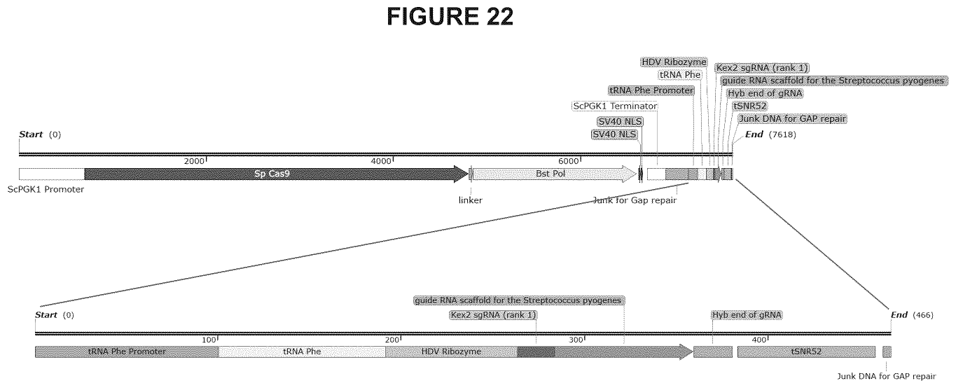

FIG. 20 depicts a schematic illustrating how the DNA insert depicted in FIG. 19 was configured to integrate into its target locus. The DNA insert (“390 DNA insert”, bottom) was 455 nucleotides in length. 3′ and 5′ regions of the DNA insert were homologous to regions of the Kex2 gene (“Wild-type Kex2 fragment in Yeast”, top) separated by a region of variable length (in this case, 95 nucleotides) that was to be deleted when the DNA insert was integrated. Between the Kex2 homology arms, the DNA insert comprised a GGGS linker (SEQ ID NO: 63) in-frame with a GFP sequence. Successful insertion of the DNA insert results in deletion of 95 nucleotides of the original Kex2 sequence. “Rank 1” in the figure here illustrates the target sequence of 5′-ATCATTAGAAGAGTTACAGGGGG-3′ (SEQ ID NO: 64) targeted by the gRNA used in this example.

### Preparation of DNA Insert Template

DNA inserts denoted DNA insert 390, DNA insert 347 and DNA insert 335 were generated, respectively, by PCR amplification using single-stranded DNAs GE-390 (SEQ ID NO: 26), GE-347 (SEQ ID NO: 27), and GE-335 (SEQ ID NO: 28) and Q5U High-Fidelity 2× Master Mix (NEB). All three insert templates were generated with the same pair of uracil-including primers: GE-328 (SEQ ID NO: 29) and GE-348 (SEQ ID NO: 30). PCR products were SPRI purified and digested with Thermolabile USER® II Enzyme (NEB) accordingly to the manufacturer recommendations to generate single-stranded regions.

### Yeast Electroporation (LiAc/Electroporation Method) and Genome Editing

EBY100 yeast cells (genotype MATa AGA1::GAL1-AGA1::URA3 ura3-52 trp1 leu2-delta200 his3-delta200 pep4::HIS3 prbd1.6R can1 GAL) were used for this editing experiment.

To prepare competent cells, 10 ml YPD media was inoculated with an EBY100 culture and grown overnight at 30° C. This starter culture was used to inoculate a 50-ml culture in YPD media to an absorbance of 0.3 and 600 nm. Once the 50-ml culture had entered early- to mid-log growth phase, the cells were pelleted at 2,500 g for 3 minutes at 4° C. and washed with 50 ml ice-cold water, followed by 50 ml ice-cold E buffer (Teknova). After washing, the cells, were suspended in 20 ml of 0.1M Lithium Acetate/10mM DTT and shook for 30 minutes at 30° C. Following this incubation, cells were pelleted again and washed once with 50 ml of ice-cold E buffer. Finally, the resulting cells were pelleted and resuspended in 100-200 μL to a final volume of 0.6 mL.

Each electroporation reaction used 1.5 μg gene-editing plasmid (e.g. pGE112 or pGE113) and DNA insert (0.3 μg to 5 μg) in 1-2 μL solution with 50 μL cells. The cell/DNA mixtures were aliquoted into prechilled electroporation cuvettes and kept on ice until electroporation (Bio-Rad, 0.54 kV and 25 mF without a pulse). Following electroporation, 1 mL warm YPD media was added to the cuvette, cells were transferred to a 15-ml tube with an additional 1 mL of YPD media, and cells were shaken for 1 h at 30° C. Following incubation, the cells were pelleted, resuspended in 10 mL SDCAA media, and transferred to a 50 ml tube with 15 mL SDCAA medium including pen-strep (1:100 dilution). After 48 hours, 800 μL, of each culture were pelleted and resuspended in 15 ml SDCAA media in 50 ml Falcon tub and incubated at 30° C. for 48 h. Following this recovery incubation, genomic DNA was prepared from each condition with a kit (Monarch Genomic DNA purification kit from NEB according to the manufacturer's protocol).

### Next-Generation Sequencing (NGS) Library Preparation

Focused NGS libraries were generated from each electroporation condition using three PCR amplification reactions: 1) focused pre-amplification; 2) amplification introducing frame shift (to increase sequence complexity—improving Illumina NGS quality); 3) amplification to introduce sample indices. The library size range was from 193-206 nucleotides covering the whole insert and both junctions Kex2-insert-Kex2.

The pre-amplification step was conducted using primers GE-349 (SEQ ID NO: 31) and GE-351 (SEQ ID NO: 32) using Q5 High-Fidelity 2× Master Mix (NEB) for 12 cycles, using SPRI cleaning afterward. The amplification introducing frame shift was performed using the pre-amplification product using a set of primers (GE-352 through GE-357, SEQ ID NOs: 33-38) and (GE-364 through GE-369, SEQ ID NOs: 39-44), using Q5 High-Fidelity 2× Master Mix (NEB) for 4 cycles, cleaning using SPRI afterward. The amplification to introduce sample indexes was performed using the frame-shift amplification product amplifying with primers GE-375/GE-383 (SEQ ID NOs: 45/47) for the pGE-112 plasmid and amplifying with primers GE-376/GE-384 (SEQ ID NOs: 46/48) for the pGE-113 plasmid, using Q5 High-Fidelity 2× Master Mix (NEB) for 12 cycles cleaning with SPRI afterward. The prepared libraries were then sequenced using the Illumina iSeq 100 according to manufacturer's recommendations.

### Analysis of Editing Efficiency

After genome editing using either the “Cas only method” (e.g. pGE-112 electroporation) and the “4M method” (e.g. pGE-113 electroporation) and preparation of NGS libraries as detailed above, presence and efficiency of genome edits to the Kex2 gene were assessed using the DNA insert 390.

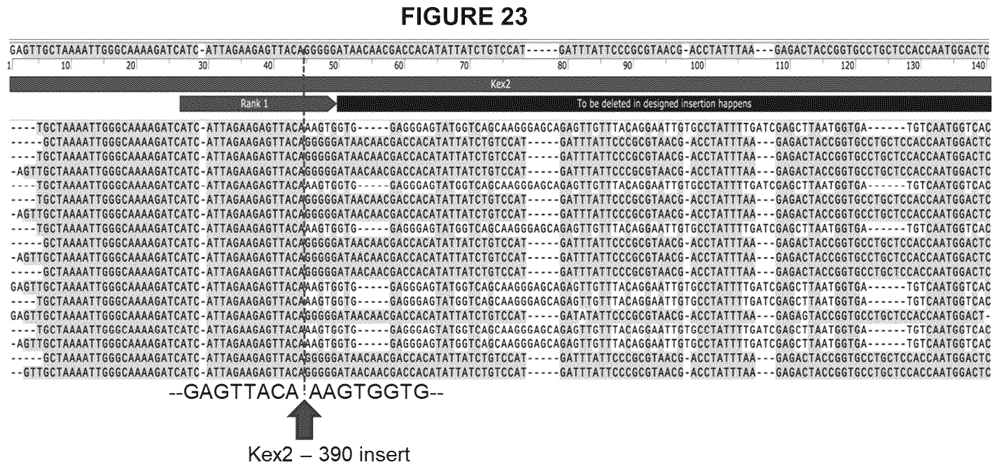

FIG. 23 summarizes an Illumina NGS analysis of this experiment, illustrating that the Cas-polymerase fusion method (“4M method”) successfully edits at the Kex2 site. The top sequence in the figure is the wild-type Kex2 sequence, while the bottom sequences are exemplary sequencing results of the genome editing condition using the Cas-polymerase fusion enzyme (“4M method”, the condition where yeast competent cells (EBY100) were electroporated with pGE113 and 390 insert DNA). The sequences shown are the vicinity of the Kex2-insert junction. Two type of sequences are observed: wild-type unrecombined sequences (sequences with fully highlighted residues) and chimeric recombined sequences including the 390 insert (sequences with highlighted and unhighlighted residues). The bar labeled “Rank 1” illustrates the gRNA targeting site (5′-ATCATTAGAAGAGTTACAGGGGG-3′ (SEQ ID NO: 64)). The Kex2-390 Insert junction (labeled in figure) shows precise insertion as predicted with no variability as illustrated by the characteristic 5′-GAGTTACA/AAGTGGTG-3′ (SEQ ID NO: 65) sequence.

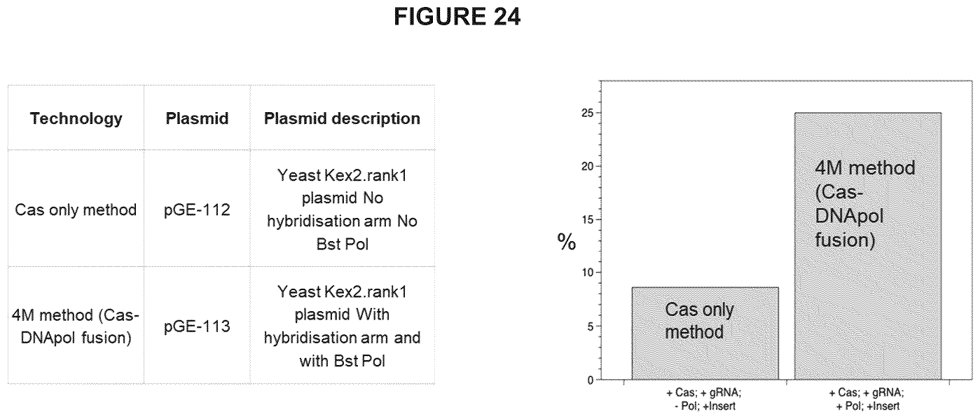

FIG. 24 illustrates editing efficiency of this experiment as assessed as assessed from the prepared and sequenced NGS libraries and compared between: (a) the “Cas only method” using electroporation of the pGE-112 plasmid into yeast, and (b) the “4M method” involving the Cas-DNApol fusion using electroporation of the pGE-113 plasmid into yeast. The left panel chart summarizes the two conditions assessed in this experiment, while the right panel graph illustrates efficiency of insertion of the DNA insert (% recombined sequence) by each method. As can be seen by the graph, the efficiency of DNA insertion is improved in the “4M method” approximately 3-fold over the “Cas only” method, indicating that the Cas-DNA polymerase fusion improves the fidelity of insertion of DNA. For the efficiency graph, efficiency was estimated using 483288 sequences for the “Cas only method” and 341994 sequences for the “4M method”.

### PCR Analysis for Leave-One-Out Reactions

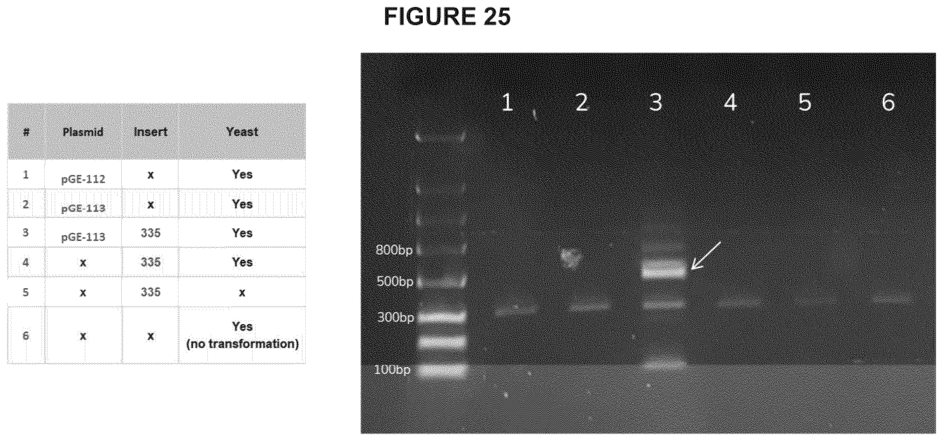

A further experiment was performed to verify the dependency of the editing reaction on the DNA-editing enzymes rather than the DNA-insert alone. Following electroporation reactions illustrated in the left panel of FIG. 25 (where the insert referred to is the 335 DNA insert), yeast Genomic DNA was prepared using Monarch Genomic DNA Purification Kit (NEB) accordingly to the manufacturer's protocol.

To investigate presence of the DNA insertion in genomic DNA PCR amplification was conducted. PCR was done on genomic DNA with one primer complementary to Kex2 sequence (GE-249, SEQ ID NO: 49), and one primer complementary to the 335 DNA insert (GE-173, SEQ ID NO: 50) to target the junction of junction Kex2 and the 335 DNA insert. The PCR reaction was performed with Q5 High-Fidelity 2× Master Mix (NEB), using SPRI cleaning afterward and analyzing using agarose-gel electrophoresis.

The right panel of FIG. 25 demonstrates that the editing reaction requires the Cas-DNApol fusion and does not proceed with the insert alone. The product corresponding to the Kex2-DNA insert junction only appears in condition 3 (see arrow), demonstrating that both Cas-DNApol fusion (provided in pGE-113) and DNA insert are required for the recombination reaction in yeast cells.

PCR and qPCR Analysis for Insert-Concentration Dependency Reactions

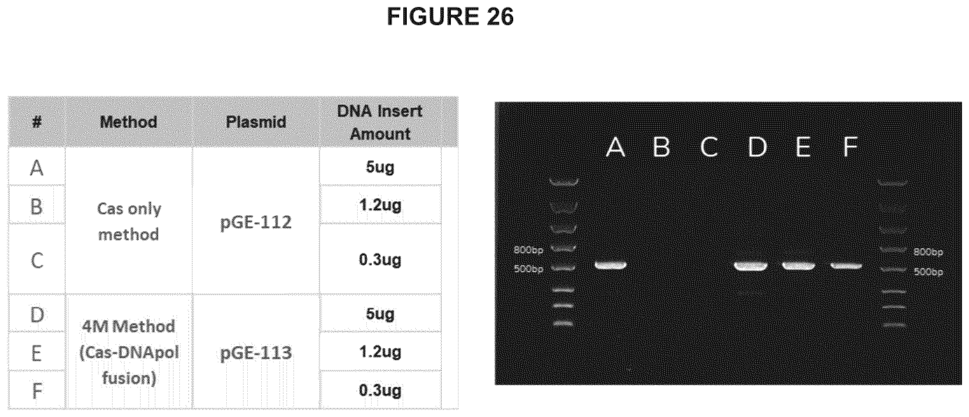

A further experiment was performed to ascertain the effect of DNA insert concentration on genomic insertion efficiency compared between the “Cas only method” and the “4M method”. Following electroporation reactions illustrated in the left panel of FIG. 26 (where the insert DNA used was the 347 DNA insert) , yeast Genomic DNA was prepared using Monarch Genomic DNA Purification Kit (NEB) accordingly to the manufacturer's protocol.

To investigate presence of the DNA insertion in genomic DNA PCR amplification was conducted. PCR was done on genomic DNA with one primer complementary to Kex2 sequence (GE-249, SEQ ID NO: 49), and one primer complementary to the 347 DNA insert (GE-173, SEQ ID NO: 50) to target the junction of junction Kex2 and the 347 DNA insert. The PCR reaction was performed with Q5 High-Fidelity 2× Master Mix (NEB), using SPRI cleaning afterward and analyzing using agarose-gel electrophoresis.

For qPCR analysis, the same reactions were performed but using a reaction including 1× Evagreen (Biotium) and sequencing on a Qiagen qPCR system with Qiagen qPCR software.

The right panel of FIG. 26 illustrates that the “4M method” is markedly less dependent on insert DNA concentration than the “Cas only method”, as can be seen by comparison of lanes A-C in the right panel (“Cas only method”) to lanes D-F (“4M method”) and the fact that recombination still occurs at the 1.2 μg and 0.3 μg insert conditions for the “4M method”, but does not occur at the 1.2 μg and 0.3 μg insert conditions for the “Cas only method.

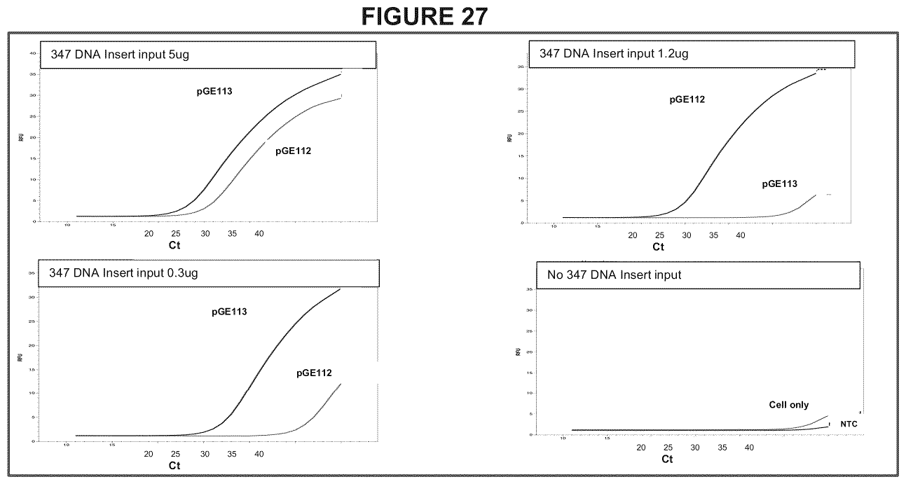

The qPCR traces in FIG. 27 illustrate that the difference in dependence on DNA insert concentration is not an artefact of gel electrophoresis. As can be seen from comparison of the curves at different insert concentrations (labeled as pGE113 for the “4M method” and pGE112 for the “Cas only method”), the difference between the methods was approximately 2 Ct at 5 μg insert DNA, approximately 15 Ct at 1.2 μg insert DNA, and approximately ˜10 Ct at 0.3 μg insert DNA.

### Embodiments

The following embodiments are not intended to be limiting in any way.
Embodiment 1. A composition comprising:
(a) a programmable nuclease configured to bind a double-stranded deoxyribonucleic acid (DNA) site; and
(b) a polypeptide with DNA polymerase activity linked to said programmable nuclease.
Embodiment 2. The composition of embodiment 1, wherein said programmable nuclease is configured to cleave at least one strand of DNA at said double-stranded DNA site.
Embodiment 3. The composition of embodiment 1 or embodiment 2, wherein said programmable nuclease is a Cas protein or a Transcription activator-like effector nuclease (TALEN).
Embodiment 4. The composition of any one of embodiments 1-3, wherein said programmable nuclease is a Cas protein, wherein said Cas protein is a Class 2, Type II Cas protein or a Class 2, Type V Cas protein.
Embodiment 5. The composition of embodiment 4, wherein said programmable nuclease comprises a Cas9 protein, a Cas12a protein, a Cas12b protein, a Cas12c protein, a Cas12d protein, a Cas12e protein, a Cas 12f protein, a C2C10 protein, a Cas14ab protein, a Type V-U1 protein, a Type V-U2 protein, a Type V-U3 protein, a Type V-U4 protein, a Type V-U5 protein, a derivative thereof, or a hybrid thereof.
Embodiment 6. The composition of any one of embodiments 4-5, wherein said composition further comprises a guide polynucleotide configured to interact with said Cas protein, wherein said guide polynucleotide is configured to hybridize to said double-stranded DNA site.
Embodiment 7. The composition of embodiment 6, wherein said guide polynucleotide comprises DNA, ribonucleic acid (RNA), or a combination thereof.
Embodiment 8. The composition of embodiment 3, wherein said programmable nuclease is a TALEN, wherein said TALEN comprises at least one Transcription activator-like effector (TAL) DNA-binding domain and an endonuclease domain.
Embodiment 9. The composition of embodiment 8, wherein said endonuclease domain comprises a FokI endonuclease domain or a PvuII endonuclease domain.
Embodiment 10. The composition of any one of embodiments 1-9, further comprising an insert DNA molecule comprising a region with complementarity to a region 5′ to said double-stranded DNA site.
Embodiment 11. The composition of embodiment 10, wherein said region with complementarity to a region 5′ to said nucleic acid site or said region with complementarity to a region 3′ to said nucleic acid site comprises at least 4 to 30 bp or 4 to 400 bp.
Embodiment 12. The composition of embodiment 11, wherein said region with homology to a region 5′ to said nucleic acid site or a region with homology to a region 3′ to said nucleic acid site, wherein said region comprises a mismatch or mutation of at least 1 bp to 5 bp.
Embodiment 13. The composition of embodiment 12, wherein said programmable nuclease is a Cas endonuclease, wherein said Cas endonuclease comprises an inactivating mutation in a single endonuclease domain.
Embodiment 14. The composition of embodiment 13, wherein said single endonuclease domain is a RuvC domain.
Embodiment 15. The composition of any one of embodiments 10-14, wherein said insert nucleic acid sequence comprises 1 bp to 20 kb.
Embodiment 16. The composition of any one of embodiments 10-15, wherein said insert DNA molecule is a single-stranded deoxyribonucleic acid molecule , at least partially a single-stranded deoxyribonucleic acid molecule, or at least partially a double-stranded deoxyribonucleic acid molecule.
Embodiment 17. The composition of embodiment 16, wherein said insert DNA molecule is: (i) linked to said programmable nuclease; (ii) linked to a guide polynucleotide configured to interact with a Cas endonuclease, wherein said programmable nuclease is a Cas enzyme; or (iii) hybridized to a guide polynucleotide configured to interact with a Cas endonuclease, wherein said programmable nuclease is a Cas enzyme.
Embodiment 18. The composition of embodiment 16, wherein said programmable nuclease is a Cas protein, wherein said composition further comprises a guide polynucleotide configured to interact with said Cas protein, wherein
(a) said guide polynucleotide further comprises a hybridization domain at a 3′ end; and
(b) wherein said insert DNA molecule comprises a first end configured to hybridize with said hybridization domain of said heterologous guide polynucleic acid.
Embodiment 19. The composition of embodiment 18, wherein said insert DNA molecule comprises said region with complementarity to a region 5′ to said double-stranded DNA site at a second end.
Embodiment 20. The composition of any one of embodiments 1-19, wherein said polypeptide with DNA polymerase activity is linked N-terminal to said programmable nuclease.
Embodiment 21. The composition of any one of embodiments 1-19, wherein said polypeptide with DNA polymerase activity is linked C-terminal to said programmable nuclease.
Embodiment 22. The composition of any one of embodiments 1-21, further comprising a linker between said programmable nuclease and said polypeptide with DNA polymerase activity.
Embodiment 23. The composition of embodiment 22, wherein said linker comprises a peptide bond, an isopeptide bond, a small molecule linker, or a combination thereof. Embodiment 24. The composition of embodiment 22, wherein said linker comprises LPXTG (SEQ ID NO: 59), GGG, (GGG)ₙ (SEQ ID NO: 60), (GGGGS)ₙ (SEQ ID NO: 61), (GGGS)ₙ (SEQ ID NO: 62), N₁₋₇, a biotin-streptavidin pair, or an analog of a biotin-streptavidin pair, or SpyTag/SpyCatcher sequences linked by an isopeptide bond.
Embodiment 25. The composition of any one of embodiments 1-24, wherein said polypeptide with DNA polymerase activity comprises a T7 DNA polymerase, Bst polymerase or an analog thereof, T4 DNA polymerase, Taq polymerase, Vent polymerase, Q5 polymerase, Klenow fragment, DNA polymerase theta, or Phi29 polymerase.
Embodiment 26. A system comprising:
(a) a class 2, type V Cas endonuclease capable of cleaving at least one strand of a DNA duplex;
(b) a polypeptide with polymerase activity linked to said Cas endonuclease;
(c) a guide polynucleotide comprising (i) a region targeting a DNA site in a cellular genome and (b) a region binding said class 2, type V Cas endonuclease, wherein said guide polynucleotide is configured to direct said class 2, type V cas endonuclease to cleave a at least one strand of DNA at a DNA site to generate a 3′ and a 5′ cleavage product;
(d) an insert DNA molecule comprising a 3′ arm capable of hybridizing with said 5′ cleavage product cleaved from said nucleic acid site in said cellular genome.
Embodiment 27. The system of embodiment 26, wherein said class 2, type V Cas endonuclease comprises a Cas12a protein, a Cas12b protein, a Cas12c protein, a Cas12d protein, a Cas12e protein, a Cas12f protein, a C2C10 protein, a Cas14ab protein, a Type V-U1 protein, a Type V-U2 protein, a Type V-U3 protein, a Type V-U4 protein, a Type V-U5 protein, a derivative thereof, or a hybrid thereof.
Embodiment 28. The system of embodiment 26 or 27, wherein said insert DNA molecule comprises an insert DNA sequence contiguous with said 3′ arm.
Embodiment 29. The system of embodiment 28, wherein said 3′ arm comprise at least 4 to 400 base pairs.
Embodiment 30. The system of embodiment 28 or 29, wherein said insert DNA sequence comprises at least 1 bp to 20 kb.
Embodiment 31. The system of any one of embodiments 26-30, wherein said insert DNA molecule is a single-stranded deoxyribonucleic acid molecule, at least partially a single-stranded deoxyribonucleic acid molecule, or at least partially a double-stranded deoxyribonucleic acid molecule.
Embodiment 32. The system of embodiment 31, wherein said insert DNA molecule is:
(i) covalently linked to said guide polynucleotide; or (ii) hybridized to said guide polynucleotide.
Embodiment 33. The system of embodiment 31, wherein
(a) said guide polynucleotide further comprises a hybridization domain at a 3′ end; and
(b) wherein said insert DNA molecule comprises a first end configured to hybridize with said hybridization domain of said heterologous guide polynucleic acid.
Embodiment 34. The system of embodiment 31, wherein
(a) said guide polynucleotide further comprises a hybridization domain at a 5′ end; and
(b) wherein said insert DNA molecule comprises a first end configured to hybridize with said hybridization domain of said heterologous guide polynucleic acid. Embodiment 35. The system of embodiment 34, wherein said class 2, type V Cas endonuclease is Cas12a.
Embodiment 36. The system of any one of embodiments 33-35, wherein said insert DNA molecule comprises said 5′ or 3′ arm at a second end.
Embodiment 37. The system of any one of embodiments 26-36, wherein said polypeptide with DNA polymerase activity is linked N-terminal to said programmable nuclease.
Embodiment 38. The system of any one of embodiments 26-36, wherein said polypeptide with DNA polymerase activity is linked C-terminal to said programmable nuclease.
Embodiment 39. The system of any one of embodiments 26-38, further comprising a linker between said programmable nuclease and said polypeptide with DNA polymerase activity.
Embodiment 40. The system of embodiment 39, wherein said linker comprises a peptide bond, an isopeptide bond, a small molecule linker, or a combination thereof.
Embodiment 41. The system of embodiment 39, wherein said linker comprises LPXTG (SEQ ID NO: 59), GGG, (GGG)ₙ (SEQ ID NO: 60), (GGGGS)ₙ (SEQ ID NO: 61), (GGGS)ₙ (SEQ ID NO: 62), N₁₋₇, a biotin-streptavidin pair, or an analog of a biotin-streptavidin pair, or SpyTag/SpyCatcher sequences linked by an isopeptide bond.
Embodiment 42. The system of any one of embodiments 26-41, wherein said polypeptide with polymerase activity has DNA polymerase activity.
Embodiment 43. The system of embodiments 42, wherein said polypeptide with DNA polymerase activity comprises a T7 DNA polymerase, Bst polymerase or an analog thereof, a T4 DNA polymerase, a Taq polymerase, a Vent polymerase, a Q5 polymerase, a Klenow fragment, a Phi29 polymerase, a functional fragment thereof, or a combination thereof.
Embodiment 44. A composition comprising:
(a) a programmable nuclease configured to bind a double-stranded DNA site; and
(b) a polypeptide having DNA topoisomerase activity linked to said programmable nuclease, wherein said polypeptide having DNA topoisomerase activity contains a catalytic hydroxyl group linked to an insert DNA template.
Embodiment 45. The composition of embodiment 44, wherein said programmable nuclease is configured to cleave at least one strand of DNA at said double-stranded DNA site.
Embodiment 46. The composition of embodiment 44 or embodiment 45, wherein said programmable nuclease is a Cas protein or a Transcription activator-like effector nuclease (TALEN).
Embodiment 47. The composition of any one of embodiments 44-46, wherein said programmable nuclease is a Cas protein, wherein said Cas protein is a Class 2, Type II Cas protein or a Class 2, Type V Cas protein.
Embodiment 48. The composition of embodiment 47, wherein said programmable nuclease comprises a Cas9 protein, a Cas12a protein, a Cas12b protein, a Cas12c protein, a Cas12d protein, a Cas12e protein, a Cas 12f protein, a C2C10 protein, a Cas14ab protein, a Type V-U1 protein, a Type V-U2 protein, a Type V-U3 protein, a Type V-U4 protein, a Type V-U5 protein, a derivative thereof, or a hybrid thereof.
Embodiment 49. The composition of any one of embodiments 47-48, wherein said composition further comprises a guide polynucleotide configured to interact with said Cas protein, wherein said guide polynucleotide is configured to hybridize to said nucleic acid site in said genome.
Embodiment 50. The composition of embodiment 49, wherein said guide polynucleotide comprises RNA or DNA.
Embodiment 51. The composition of embodiment 46, wherein said programmable nuclease is a TALEN, wherein said TALEN comprises at least one TAF effector DNA-binding domain and a FokI endonuclease domain.
Embodiment 52. The composition of any one of embodiments 44-50, further comprising an insert DNA molecule comprising a region homologous to a region 5′ to said nucleic acid site or a region homologous to a region 3′ to said nucleic acid site contiguous with an insert nucleic acid sequence.
Embodiment 53. The composition of embodiment 52, wherein said region homologous to a region 5′ to said nucleic acid site or said region homologous to a region 3′ to said nucleic acid site comprises at least 4 base pairs to 400 base pairs.
Embodiment 54. The composition of any one of embodiments 52-53, wherein said insert nucleic acid sequence comprises 1 base pair to 20kb.
Embodiment 55. The composition of any one of embodiments 52-54, wherein said insert DNA molecule is a single-stranded deoxyribonucleic acid molecule , at least partially a single-stranded deoxyribonucleic acid molecule, or at least partially a double-stranded deoxyribonucleic acid molecule.
Embodiment 56. The composition of embodiment 55, wherein said insert DNA molecule is linked to said catalytic hydroxyl group of said polypeptide having DNA topoisomerase activity at a first end, and wherein said insert DNA molecule comprises said region homologous to a region 5′ to said nucleic acid site or said region homologous to a region 3′ to said nucleic acid site at a second end.
Embodiment 57. The composition of any one of embodiments 44-56, wherein said polypeptide having DNA topoisomerase activity is linked N-terminal to said programmable nuclease.
Embodiment 58. The composition of any one of embodiments 44-56, wherein said polypeptide having DNA topoisomerase activity is linked C-terminal to said programmable nuclease.
Embodiment 59. The composition of any one of embodiments 44-58, further comprising a linker between said programmable nuclease and said polypeptide having DNA topoisomerase activity.
Embodiment 60. The composition of embodiment 59, wherein said linker comprises a peptide bond, an isopeptide bond, a small molecule linker, or a combination thereof. Embodiment 61. The composition of embodiment 59, wherein said linker comprises LPXTG (SEQ ID NO: 59), GGG, (GGG)ₙ (SEQ ID NO: 60), (GGGGS)ₙ (SEQ ID NO: 61), (GGGS)ₙ (SEQ ID NO: 62), N₁₋₇, a biotin-streptavidin pair, or an analog of a biotin-streptavidin pair, or SpyTag/SpyCatcher sequences linked by an isopeptide bond.
Embodiment 62. The composition of any one of embodiments 44-61, wherein said polypeptide having DNA topoisomerase activity comprises a Type I topoisomerase or a Type II topoisomerase.
Embodiment 63. The composition of embodiment 62, comprising a Type I topoisomerase, wherein said Type I topoisomerase comprises a Type 1A topoisomerase.
Embodiment 64. The composition of embodiment 63, comprising a Type 1A topoisomerase, wherein said Type 1A topoisomerase comprises E. coli Eubacterial DNA topoisomerase I, E. coli Eubacterial DNA topoisomerase III, S. cerevisiae Yeast DNA topoisomerase IIII, H. sapiens DNA topoisomerase IIIα or IIIβ, S. acidocaldarius eubacterial and archaeal reverse DNA gyrase, or M. kandleri eubacterial reverse gyrase.
Embodiment 65. The composition of embodiment 62, comprising a Type I topoisomerase, wherein said Type I topoisomerase comprises a Type 1B topoisomerase.
Embodiment 66. The composition of embodiment 65, comprising a Type 1B topoisomerase, wherein said Type 1B topoisomerase comprises H. sapiens eukaryotic DNA topoisomerase I, Vaccinia poxvirus topoisomerase I, or M. kandleri hyperthermophilic eubacterial DNA topoisomerase V.
Embodiment 67. The composition of embodiment 62, comprising a Type II topoisomerase, wherein said Type II topoisomerase comprises a Type IIA topoisomerase.
Embodiment 68. The composition of embodiment 67, comprising a Type IIA topoisomerase, wherein said Type IIA topoisomerase comprises E. coli eubacterial DNA gyrase, E. coli eubacterial DNA topoisomerase IV, S. cerevisiae yeast DNA topoisomerase II, or H. sapiens mammalian DNA topoisomerase IIα or IIβ.
Embodiment 69. The composition of embodiment 62, comprising a Type II topoisomerase, wherein said Type II topoisomerase comprises a Type IIB topoisomerase.
Embodiment 70. The composition of embodiment 69, comprising a Type IIB topoisomerase, wherein said Type IIB topoisomerase comprises S. shibatae archaeal DNA topoisomerase VI.
Embodiment 71. A composition comprising a complex having the following linked components:
(a) a polynucleotide comprising a region homologous to a nucleic acid site in a cellular genome;
(b) a displacement annealing domain comprising:
   (i) a polypeptide having RecA-like activity; and
   (ii) at least one polypeptide having RecN-like activity; and
(c) a polypeptide with DNA polymerase activity.
Embodiment 72. The composition of embodiment 71, wherein said displacement annealing domain comprises from N- to C-terminus: at least one first polypeptide with RecA-like activity, an optional first linker, a polypeptide having RecN-like activity, an optional second linker, and at least one second polypeptide having RecA-like activity.
Embodiment 73. The method of embodiment 71 or 72, wherein said at least one first polypeptide with RecA-like activity comprises at least two, at least three, at least four, or at least five polypeptides with Rec-A like activity.
Embodiment 74. The method of embodiment 71 or 72, wherein said at least one second polypeptide with RecA-like activity comprises at least two, at least three, at least four, or at least five polypeptides with Rec-A like activity.
Embodiment 75. The composition of any one of embodiments 71-74, wherein said complex comprises the following polypeptides from N- to C-terminus: a polypeptide with Rec A-like activity, a polypeptide with RecN-like activity, a polypeptide with RecA-like activity, and a polypeptide with DNA polymerase activity.
Embodiment 76. The composition of any one of embodiments 71-75, wherein said polypeptide with RecA-like activity is RecA from E. coli or Rad54 from H. sapiens.
Embodiment 77. The composition of any one of embodiments 71-76, wherein said polypeptide with RecN-like activity is RecN from E. coli or Rad51 from H. sapiens.
Embodiment 78. The composition of any one of embodiments 71-77, wherein said polypeptide with DNA polymerase activity comprises a T7 DNA polymerase, Bst polymerase or an analog thereof, a T4 DNA polymerase, a Taq polymerase, a Vent polymerase, a Q5 polymerase, a Klenow fragment, a Phi29 polymerase, a functional fragment thereof, or any combination thereof.
Embodiment 79. The composition of any one of embodiments 71-78, wherein said polynucleotide comprising said region homologous to said nucleic acid site in said cellular genome is linked to an N-terminus or C-terminus of said displacement annealing domain.
Embodiment 80. The composition of any one of embodiments 71-78, wherein said polynucleotide comprising said region homologous to said nucleic acid site in said cellular genome is linked to an N-terminus or a C-terminus of said polypeptide with DNA polymerase activity.
Embodiment 81. The composition of any one of embodiments 71-80, wherein said region homologous to a nucleic acid site in a cellular genome comprises at least 10, at least 20, at least 30, at least 40, or at least 50 base pairs.
Embodiment 82. The composition of any one of embodiments 71-81, wherein said polynucleotide comprising a region homologous to a nucleic acid site in a cellular genome further comprises an insert nucleic acid sequence comprising at least about 1 bp to at least about 20 kb.
Embodiment 83. A composition comprising a fusion protein comprising:
(a) a programmable nuclease configured to bind a double-stranded deoxyribonucleic acid (DNA) site; and
(b) a polypeptide with DNA polymerase activity linked to said programmable nuclease.
Embodiment 84. The composition of embodiment 83, wherein said programmable nuclease is configured to cleave at least one strand of DNA at said double-stranded DNA site.
Embodiment 85. The composition of embodiment 83 or embodiment 84, wherein said programmable nuclease is a Cas protein or a Transcription activator-like effector nuclease (TALEN).
Embodiment 86. The composition of any one of embodiments 83-85, wherein said programmable nuclease is a Cas protein, wherein said Cas protein is a Class 2, Type II Cas protein or a Class 2, Type V Cas protein.
Embodiment 87. The composition of embodiment 86, wherein said programmable nuclease comprises a Cas9 protein, a Cas12a protein, a Cas12b protein, a Cas12c protein, a Cas12d protein, a Cas12e protein, a Cas 12f protein, a C2C10 protein, a Cas14ab protein, a Type V-U1 protein, a Type V-U2 protein, a Type V-U3 protein, a Type V-U4 protein, a Type V-U5 protein, a derivative thereof, or a hybrid thereof.
Embodiment 88. The composition of any one of embodiments 86-87, wherein said composition further comprises a guide polynucleotide configured to interact with said Cas protein, wherein said guide polynucleotide is configured to hybridize to said double-stranded DNA site.
Embodiment 89. The composition of embodiment 88, wherein said guide polynucleotide comprises DNA, ribonucleic acid (RNA), or a combination thereof.
Embodiment 90. The composition of embodiment 85, wherein said programmable nuclease is a TALEN, wherein said TALEN comprises at least one transcription activator-like effector (TAL) DNA-binding domain and an endonuclease domain.
Embodiment 91. The composition of embodiment 90, wherein said endonuclease domain comprises a FokI endonuclease domain or a PvuII endonuclease domain.
Embodiment 92. The composition of any one of embodiments 83-91, further comprising an insert DNA molecule comprising a region with complementarity to a region 5′ to said double-stranded DNA site or a region with complementarity to a region 3′ to said nucleic acid site.
Embodiment 93. The composition of embodiment 92, wherein said region with complementarity to a region 5′ to said nucleic acid site or said region with complementarity to a region 3′ to said nucleic acid site comprises at least 4 to 30 bp or at least 4 to 400 bp.
Embodiment 94. The composition of embodiment 93, wherein said region with complementarity to a region 5′ to said nucleic acid site or a region with complementarity to a region 3′ to said nucleic acid site, wherein said region comprises a mismatch or mutation of at least 1 bp to at least 5 bp.
Embodiment 95. The composition of embodiment 94, wherein said programmable nuclease is a Cas endonuclease, wherein said Cas endonuclease comprises an inactivating mutation in a single endonuclease domain.
Embodiment 96. The composition of embodiment 13, wherein said single endonuclease domain is a RuvC domain.
Embodiment 97. The composition of any one of embodiments 92-96, wherein said insert nucleic acid sequence comprises at least about 1 bp to at least about 20 kb.
Embodiment 98. The composition of any one of embodiments 92-97, wherein said insert DNA molecule is a single-stranded deoxyribonucleic acid molecule , at least partially a single-stranded deoxyribonucleic acid molecule, or at least partially a double-stranded deoxyribonucleic acid molecule.
Embodiment 99. The composition of any one of embodiments 92-97, wherein said insert DNA molecule is a double-stranded deoxyribonucleic acid molecule.
Embodiment 100. The composition of embodiment 98, wherein said insert DNA molecule is at least partially a double-stranded deoxyribonucleic acid molecule, wherein said insert DNA molecule comprises a single-stranded region at a 3′ end and a single-stranded region at a 5′ end.
Embodiment 101. The composition of embodiment 98, wherein said insert DNA molecule is: (i) linked to said programmable nuclease; (ii) linked to a guide polynucleotide configured to interact with a Cas endonuclease, wherein said programmable nuclease is a Cas enzyme; or (iii) hybridized to a guide polynucleotide configured to interact with a Cas endonuclease, wherein said programmable nuclease is a Cas enzyme.
Embodiment 102. The composition of embodiment 98, wherein said programmable nuclease is a Cas protein, wherein said composition further comprises a guide polynucleotide configured to interact with said Cas protein, wherein
(a) said guide polynucleotide further comprises a hybridization domain at a 3′ end; and
(b) wherein said insert DNA molecule comprises a first end configured to hybridize with said hybridization domain of said guide polynucleotide at said 3′ end of said insert DNA.
Embodiment 103. The composition of embodiment 102, wherein said insert DNA molecule comprises a region with complementarity to a region 5′ to said double-stranded DNA site at said 5′ end of said insert DNA.
Embodiment 104. The composition of any one of embodiments 83-103, wherein said polypeptide with DNA polymerase activity is linked N-terminal to said programmable nuclease.
Embodiment 105. The composition of any one of embodiments 83-103, wherein said polypeptide with DNA polymerase activity is linked C-terminal to said programmable nuclease.
Embodiment 106. The composition of any one of embodiments 83-105, further comprising a linker between said programmable nuclease and said polypeptide with DNA polymerase activity.
Embodiment 107. The composition of embodiment 106, wherein said linker comprises a peptide bond, an isopeptide bond, a small molecule linker, or a combination thereof.
Embodiment 108. The composition of embodiment 106, wherein said linker comprises LPXTG (SEQ ID NO: 59), GGG, (GGG)ₙ (SEQ ID NO: 60), (GGGGS)ₙ (SEQ ID NO: 61), (GGGS)ₙ (SEQ ID NO: 62), N₁₋₇, a biotin-streptavidin pair, or an analog of a biotin-streptavidin pair, or SpyTag/SpyCatcher sequences linked by an isopeptide bond.
Embodiment 109. The composition of any one of embodiments 83-108, wherein said polypeptide with DNA polymerase activity comprises a T7 DNA polymerase, Bst polymerase or an analog thereof, T4 DNA polymerase, Taq polymerase, Vent polymerase, Q5 polymerase, Klenow fragment, DNA polymerase theta, or Phi29 polymerase.
Embodiment 110. The composition of any one of embodiments 83-108, wherein said polypeptide with DNA polymerase activity comprises a sequence having at least 80%, at least 85%, at least 86%, at least 87%, at least 88%, at least 89%, at least 90%, at least 91%, at least 92%, at least 93%, at least 94%, at least 95%, at least 96%, at least 97%, or at least 99% sequence identity to any one of SEQ ID NOs: 16, 26, 51, 52, 53, 54, 55, 56, 57, or 58, or a variant thereof.
Embodiment 111. The composition of any one of embodiments 86-89 or 92-110, wherein said Cas protein comprises a sequence having at least 80%, at least 85%, at least 86%, at least 87%, at least 88%, at least 89%, at least 90%, at least 91%, at least 92%, at least 93%, at least 94%, at least 95%, at least 96%, at least 97%, or. at least 99% sequence identity to any one of SEQ ID NOs: 14 or 15, or a variant thereof.
Embodiment 112. The composition of any one of embodiments 86-89 or 92-110, wherein said fusion protein comprises a sequence having at least 80%, at least 85%, at least 86%, at least 87%, at least 88%, at least 89%, at least 90%, at least 91%, at least 92%, at least 93%, at least 94%, at least 95%, at least 96%, at least 97%, or at least 99% sequence identity to SEQ ID NO: 26 or a variant thereof.
Embodiment 113. A system comprising the composition of any one of embodiments 83-112.
Embodiment 114. A nucleic acid sequence encoding the fusion protein of any one of embodiments 83-112.
Embodiment 115. A method of editing a double stranded DNA site in a cell, comprising introducing to said cell the composition of any one of embodiments 83-112.
Embodiment 116. A method of editing a double-stranded DNA site in a cell, comprising introducing to said cell:
(a) a fusion protein comprising: (i) a programmable nuclease configured to bind a double-stranded DNA site wherein said programmable nuclease is a Cas protein; and (ii) a polypeptide with DNA polymerase activity linked to said programmable nuclease;
(b) a guide polynucleotide configured to interact with said Cas protein and configured to target said genomic locus; and
(c) an insert DNA molecule comprising a region with complementarity to a region 5′ to said double-stranded DNA site or a region with complementarity to a region 3′ to said nucleic acid site.
Embodiment 117. The method of embodiment 116, wherein said region with complementarity to a region 5′ to said nucleic acid site or said region with complementarity to a region 3′ to said nucleic acid site comprises at least 4 to 30 bp or at least 4 to 400 bp
Embodiment 118. The method of embodiment 117, wherein said region with complementarity to a region 5′ to said nucleic acid site or a region with complementarity to a region 3′ to said nucleic acid site, wherein said region comprises a mismatch or mutation of at least 1 bp to at least 5 bp.
Embodiment 119. The method of any one of embodiments 116-118, wherein said Cas protein is a Class 2, Type II Cas protein or a Class 2, Type V Cas protein.
Embodiment 120. The method of embodiment 119, wherein said programmable nuclease comprises a Cas9 protein, a Cas12a protein, a Cas12b protein, a Cas12c protein, a Cas12d protein, a Cas12e protein, a Cas 12f protein, a C2C10 protein, a Cas14ab protein, a Type V-U1 protein, a Type V-U2 protein, a Type V-U3 protein, a Type V-U4 protein, a Type V-U5 protein, a derivative thereof, or a hybrid thereof.
Embodiment 121. The method of any one of embodiments 116-120, wherein said guide polynucleotide comprises DNA, RNA, or a combination thereof.
Embodiment 122. The method of any one of embodiments 116-121, wherein said insert DNA molecule is a single-stranded deoxyribonucleic acid molecule, at least partially a single-stranded deoxyribonucleic acid molecule, or at least partially a double-stranded deoxyribonucleic acid molecule.
Embodiment 123. The method of any one of embodiments 116-121, wherein said insert DNA molecule is a double-stranded deoxyribonucleic acid molecule.
Embodiment 124. The method of any one of embodiments 116-121, wherein said insert DNA molecule is at least partially a double-stranded deoxyribonucleic acid molecule,
wherein said insert DNA molecule comprises a single-stranded region at a 3′ end and a single-stranded region at a 5′ end.
Embodiment 125. The method of embodiment 124, wherein
(a) said guide polynucleotide further comprises a hybridization domain at a 3′ end; and
(b) wherein said insert DNA molecule comprises a first end configured to hybridize with said hybridization domain of said guide polynucleotide at said 3′ end of said insert DNA.
Embodiment 126. The method of embodiment 125, wherein said insert DNA molecule comprises a region with complementarity to a region 5′ to said double-stranded DNA site at said 5′ end of said insert DNA.
Embodiment 127. The method of any one of embodiments 116-126, said polypeptide with DNA polymerase activity is linked N-terminal to said programmable nuclease. Embodiment 128. The method of any one of embodiments 116-126, wherein said polypeptide with DNA polymerase activity is linked C-terminal to said programmable nuclease.
Embodiment 129. The method of any one of embodiments 116-128, further comprising a linker between said programmable nuclease and said polypeptide with DNA polymerase activity
Embodiment 130. The method of any one of embodiments 116-129, wherein said polypeptide with DNA polymerase activity comprises a T7 DNA polymerase, Bst polymerase or an analog thereof, T4 DNA polymerase, Taq polymerase, Vent polymerase, Q5 polymerase, Klenow fragment, DNA polymerase theta, or Phi29 polymerase.
Embodiment 131. The method of any one of embodiments 116-129, wherein said polypeptide with DNA polymerase activity comprises a sequence having at least 80%, at least 85%, at least 86%, at least 87%, at least 88%, at least 89%, at least 90%, at least 91%, at least 92%, at least 93%, at least 94%, at least 95%, at least 96%, at least 97%, or at least 99% sequence identity to any one of SEQ ID NOs: 16, 26, 51, 52, 53, 54, 55, 56, 57, or 58, or a variant thereof.
Embodiment 132. The method of any one of embodiments 116-131, wherein said Cas protein comprises a sequence having at least 80%, at least 85%, at least 86%, at least 87%, at least 88%, at least 89%, at least 90%, at least 91%, at least 92%, at least 93%, at least 94%, at least 95%, at least 96%, at least 97%, or. at least 99% sequence identity to any one of SEQ ID NOs: 14 or 15, or a variant thereof.
Embodiment 133. The method of any one of embodiments 116-132, wherein said method is at least about 3-times effective for introducing said DNA insert to said genomic locus, compared to said method using only a Cas protein without a polypeptide with DNA polymerase activity.
Embodiment 134. The method of any one of embodiments 116-133, wherein said method has at least about 10%, at least about 15%, at least about 20%, or at least about 25% efficiency for integration of said DNA insert to said genomic locus.
Embodiment 135. The method of any one of embodiments 116-134, wherein said cell is a prokaryotic cell.
Embodiment 136. The method of any one of embodiments 116-134, wherein said cell is a eukaryotic cell.
Embodiment 137. The method of embodiment 136, wherein said cell is a yeast cell.
Embodiment 138. The method of embodiment 136, wherein said cell is a human cell.
Embodiment 139. The method of any one of embodiments 115-138, wherein introducing to said cell further comprises contacting said cell with a nucleic acid or vector encoding said fusion protein or said guide polynucleotide.
Embodiment 140. The method of embodiment 115-138, wherein introducing to said cell further comprises contacting said cell with a ribonucleoprotein complex (RNP) comprising said fusion protein or said guide polynucleotide.
Embodiment 141. A vector comprising or encoding said composition of any one of embodiments 83-112.
Embodiment 142. A host cell comprising said vector of embodiment 141.
Embodiment 143. The host cell of embodiment 142, wherein said cell is a prokaryotic cell
Embodiment 144. The host cell of embodiment 142, wherein said cell is a eukaryotic cell.
Embodiment 145. The host cell of embodiment 144, wherein said cell is a yeast cell or a human cell.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-63. (canceled)

64. A method of editing a double-stranded DNA site in a cell, comprising introducing to said cell:
(a) a fusion protein comprising: (i) a programmable nuclease configured to bind a double-stranded DNA site wherein said programmable nuclease is a Cas protein; and (ii) a polypeptide with DNA polymerase activity linked to said programmable nuclease;
(b) a guide polynucleotide configured to interact with said Cas protein and configured to target said double-stranded DNA site; and
(c) an insert DNA molecule comprising a region with complementarity to a region 5′ to said double-stranded DNA site or a region with complementarity to a region 3′ to said double-stranded DNA site.

65. The method of claim 64, wherein said region with complementarity to said region 5′ to said double-stranded DNA site or said region with complementarity to said region 3′ to said double-stranded DNA site comprises at least 4 to 30 bp.

66. The method of claim 65, wherein said region with complementarity to said region 5′ to said double-stranded DNA site or said region with complementarity to said region 3′ to said double-stranded DNA site comprises a mismatch or mutation of at least 1 bp to at least 5 bp.

67. The method of claim 64, wherein said Cas protein is: (i) a Class 2, Type II Cas protein; or (ii) a Class 2, Type V Cas protein.

68. The method of claim 67, wherein said Cas protein comprises said Class 2, Type V Cas protein, and said Class 2, Type V Cas protein further comprises a Cas12c protein, a Cas12d protein, a Cas12e protein, or a Cas 12f protein.

69. The method of claim 64, wherein said guide polynucleotide further comprises RNA or a combination of DNA and RNA.

70. The method of claim 64, wherein said insert DNA molecule is at least partially a double-stranded DNA molecule.

71. The method of claim 64, wherein said insert DNA molecule is at least partially a double-stranded DNA molecule, wherein said insert DNA molecule further comprises a single-stranded region at a 3′ end and a single-stranded region at a 5′ end.

72. The method of claim 71, wherein
(a) said guide polynucleotide further comprises a hybridization domain at a 3′ end; and
(b) wherein said insert DNA molecule comprises a first end configured to hybridize with said hybridization domain of said guide polynucleotide at said 3′ end of said insert DNA.

73. The method of claim 72, wherein said insert DNA molecule further comprises a region with complementarity to a region 5′ to said double-stranded DNA site at said 5′ end of said insert DNA.

74. The method of claim 64, wherein said polypeptide with DNA polymerase activity has DNA-dependent DNA polymerase activity.

75. The method of claim 64, wherein said polypeptide with DNA polymerase activity is linked C-terminal to said programmable nuclease.

76. The method of claim 64, further comprising a linker between said programmable nuclease and said polypeptide with DNA polymerase activity.

77. The method of claim 74, wherein said polypeptide with DNA-dependent DNA polymerase activity comprises a T7 DNA polymerase, Bst polymerase or an analog thereof, T4 DNA polymerase, Taq polymerase, Vent polymerase, Q5 polymerase, Klenow fragment, DNA polymerase theta, or Phi29 polymerase.

78. The method of claim 74, wherein said polypeptide with DNA-dependent DNA polymerase activity further comprises a sequence having at least 80% sequence identity to any one of SEQ ID NOs: 16, 26, 51, 52, 53, 54, 55, 56, 57, or 58, or a variant thereof.

79. The method of claim 64, wherein said Cas protein further comprises a sequence having at least 80% sequence identity to any one of SEQ ID NOs: 14 or 15, or a variant thereof.

80. The method of claim 64, wherein said method is at least about 3-times more effective for introducing said DNA insert to said genomic locus, compared to said method using only a Cas protein without a polypeptide with DNA polymerase activity.

81. The method of claim 64, wherein said cell is a eukaryotic cell.

82. The method of claim 64, further comprising contacting said cell with a ribonucleoprotein (RNP) complex comprising said fusion protein or said guide polynucleotide.

83. The method of claim 64, wherein said Cas protein is configured to cleave both strands of DNA at said double-stranded DNA site.

84. The method of claim 64, wherein said Cas protein does not comprise an inactivating mutation in an endonuclease domain.
